SEQUENCE LISTING (541 sequences)

SEQ ID NO: 1 - protein, 15 aa, synthetic construct
  REGION 1..15: note = Description of Artificial Sequence: Synthetic peptide
QVSHWVSGLAEGSFG

SEQ ID NO: 2 - protein, 15 aa, synthetic construct
  REGION 1..15: note = Description of Artificial Sequence: Synthetic peptide
LSHTSGRVEGSVSLL

SEQ ID NO: 3 - protein, 10 aa, synthetic construct
  REGION 1..10: note = Description of Artificial Sequence: Synthetic peptide
QMWAPQWGPD

SEQ ID NO: 4 - protein, 11 aa, synthetic construct
  REGION 1..11: note = Description of Artificial Sequence: Synthetic peptide
MASMTGGQQMG

SEQ ID NO: 5 - protein, 10 aa, synthetic construct
  REGION 1..10: note = Description of Artificial Sequence: Synthetic peptide
EQKLISEEDL

SEQ ID NO: 6 - protein, 9 aa, synthetic construct
  REGION 1..9: note = Description of Artificial Sequence: Synthetic peptide
YPYDVPDYA

SEQ ID NO: 7 - protein, 11 aa, synthetic construct
  REGION 1..11: note = Description of Artificial Sequence: Synthetic peptide
YTDIEMNRLGK

SEQ ID NO: 8 - protein, 15 aa, synthetic construct
  REGION 1..15: note = Description of Artificial Sequence: Synthetic peptide
KETAAAKFERQHMDS

SEQ ID NO: 9 - protein, 14 aa, synthetic construct
  REGION 1..14: note = Description of Artificial Sequence: Synthetic peptide
GKPIPNPLLGLDST

SEQ ID NO: 10 - protein, 8 aa, synthetic construct
  REGION 1..8: note = Description of Artificial Sequence: Synthetic peptide
DYKDDDDK

SEQ ID NO: 11 - protein, 13 aa, synthetic construct
  REGION 1..13: note = Description of Artificial Sequence: Synthetic peptide
GAPVPYPDPLEPR

SEQ ID NO: 12 - protein, 6 aa, synthetic construct
  REGION 1..6: note = Description of Artificial Sequence: Synthetic peptide
HHHHHH

SEQ ID NO: 13 - protein, 18 aa, synthetic construct
  REGION 1..18: note = Description of Artificial Sequence: Synthetic peptide
TKENPRSNQEESYDDNES

SEQ ID NO: 14 - protein, 8 aa, synthetic construct
  REGION 1..8: note = Description of Artificial Sequence: Synthetic peptide
WSHPQFEK

SEQ ID NO: 15 - protein, 12 aa, synthetic construct
  REGION 1..12: note = Description of Artificial Sequence: Synthetic peptide
PDRVRAVSHWSS

SEQ ID NO: 16-17 - skipped (2 entries)

SEQ ID NO: 18 - protein, 28 aa, synthetic construct
  REGION 1..28: note = Description of Artificial Sequence: Synthetic peptide
  REGION 1..28: note = See specification as filed for detailed description of substitutions and preferred embodiments
  SITE 2: note = Lys, Cys or an Azido-containing amino acid
AXDDQNPWRAYLDLLFPTDTLLLDLLWA

SEQ ID NO: 19 - skipped

SEQ ID NO: 20 - protein, 36 aa, synthetic construct
  REGION 1..36: note = Description of Artificial Sequence: Synthetic polypeptide
  REGION 1..36: note = See specification as filed for detailed description of substitutions and preferred embodiments
  SITE 2: note = Lys, Cys or an Azido-containing amino acid
AXEQNPIYWARYADWLFTTPLLLLDLALLVDADEGT

SEQ ID NO: 21 - protein, 22 aa, synthetic construct
  REGION 1..22: note = Description of Artificial Sequence: Synthetic peptide
WARYADWLFTTPLLLLDLALLV

SEQ ID NO: 22 - protein, 36 aa, synthetic construct
  REGION 1..36: note = Description of Artificial Sequence: Synthetic polypeptide
GGEQNPIYWARYADWLFTTPLLLLDLALLVDADEGT

SEQ ID NO: 23 - protein, 35 aa, synthetic construct
  REGION 1..35: note = Description of Artificial Sequence: Synthetic polypeptide
AEQNPIYWARYADWLFTTPLLLLDLALLVDADEGT

SEQ ID NO: 24 - protein, 32 aa, synthetic construct
  REGION 1..32: note = Description of Artificial Sequence: Synthetic polypeptide
ADDQNPWRAYLDLLFPTDTLLLDLLWDADECG

SEQ ID NO: 25 - protein, 32 aa, synthetic construct
  REGION 1..32: note = Description of Artificial Sequence: Synthetic polypeptide
ACDDQNPWRAYLDLLFPTDTLLLDLLWDADEG

SEQ ID NO: 26 - protein, 32 aa, synthetic construct
  REGION 1..32: note = Description of Artificial Sequence: Synthetic polypeptide
AKDDQNPWRAYLDLLFPTDTLLLDLLWDADEG

SEQ ID NO: 27 - protein, 38 aa, synthetic construct
  REGION 1..38: note = Description of Artificial Sequence: Synthetic polypeptide
AAEQNPIYWARYADWLFTTPLLLLDLALLVDADEGTCG

SEQ ID NO: 28 - protein, 36 aa, synthetic construct
  REGION 1..36: note = Description of Artificial Sequence: Synthetic polypeptide
AEQNPIYWARYADWLFTTPLLLLDLALLVDADEGCT

SEQ ID NO: 29 - protein, 38 aa, synthetic construct
  REGION 1..38: note = Description of Artificial Sequence: Synthetic polypeptide
GGEQNPIYWARYADWLFTTPLLLLDLALLVDADEGTCG

SEQ ID NO: 30 - protein, 37 aa, synthetic construct
  REGION 1..37: note = Description of Artificial Sequence: Synthetic polypeptide
ACEQNPIYWARYADWLFTTPLLLLDLALLVDADEGTG

SEQ ID NO: 31 - protein, 36 aa, synthetic construct
  REGION 1..36: note = Description of Artificial Sequence: Synthetic polypeptide
ACEQNPIYWARYADWLFTTPLLLLDLALLVDADEGT

SEQ ID NO: 32 - protein, 36 aa, synthetic construct
  REGION 1..36: note = Description of Artificial Sequence: Synthetic polypeptide
AKEQNPIYWARYADWLFTTPLLLLDLALLVDADEGT

SEQ ID NO: 33 - protein, 37 aa, synthetic construct
  REGION 1..37: note = Description of Artificial Sequence: Synthetic polypeptide
AKEQNPIYWARYADWLFTTPLLLLDLALLVDADEGTG

SEQ ID NO: 34 - protein, 39 aa, synthetic construct
  REGION 1..39: note = Description of Artificial Sequence: Synthetic polypeptide
AAEQNPIYWARYADWLFTTPLLLLDLALLVDADEGTKCG

SEQ ID NO: 35 - protein, 36 aa, synthetic construct
  REGION 1..36: note = Description of Artificial Sequence: Synthetic polypeptide
AKEQNPIYWARYADWLFTTPLLLLDLALLVDADECT

SEQ ID NO: 36 - protein, 37 aa, synthetic construct
  REGION 1..37: note = Description of Artificial Sequence: Synthetic polypeptide
ACEQNPIYWARYANWLFTTPLLLLNLALLVDADEGTG

SEQ ID NO: 37 - protein, 36 aa, synthetic construct
  REGION 1..36: note = Description of Artificial Sequence: Synthetic polypeptide
ACEQNPIYWARYANWLFTTPLLLLNLALLVDADEGT

SEQ ID NO: 38 - protein, 37 aa, synthetic construct
  REGION 1..37: note = Description of Artificial Sequence: Synthetic polypeptide
ACEQNPIYWARYAKWLFTTPLLLLKLALLVDADEGTG

SEQ ID NO: 39 - protein, 36 aa, synthetic construct
  REGION 1..36: note = Description of Artificial Sequence: Synthetic polypeptide
GGEQNPIYWARYADWLFTTPLLLLDLALLVNANQGT

SEQ ID NO: 40 - protein, 36 aa, synthetic construct
  REGION 1..36: note = Description of Artificial Sequence: Synthetic polypeptide
AAEQNPIYWARYADWLFTTPLLLLALALLVDADEGT

SEQ ID NO: 41 - protein, 39 aa, synthetic construct
  REGION 1..39: note = Description of Artificial Sequence: Synthetic polypeptide
AAEQNPIYWARYADWLFTTPLLLLELALLVDADEGTKCG

SEQ ID NO: 42 - protein, 36 aa, synthetic construct
  REGION 1..36: note = Description of Artificial Sequence: Synthetic polypeptide
AAEQNPIYWARYAAWLFTTPLLLLDLALLVDADEGT

SEQ ID NO: 43 - protein, 36 aa, synthetic construct
  REGION 1..36: note = Description of Artificial Sequence: Synthetic polypeptide
AAEQNPIYWARYADWLFTTALLLLDLALLVDADEGT

SEQ ID NO: 44 - protein, 36 aa, synthetic construct
  REGION 1..36: note = Description of Artificial Sequence: Synthetic polypeptide
AAEQNPIYWARYADWLFTTPLLLLELALLVDADEGT

SEQ ID NO: 45 - protein, 36 aa, synthetic construct
  REGION 1..36: note = Description of Artificial Sequence: Synthetic polypeptide
AAEQNPIYWARYAEWLFTTPLLLLDLALLVDADEGT

SEQ ID NO: 46 - protein, 39 aa, synthetic construct
  REGION 1..39: note = Description of Artificial Sequence: Synthetic polypeptide
AAEQNPIIYWARYADWLFTDLPLLLLDLLALLVDADEGT

SEQ ID NO: 47 - protein, 37 aa, synthetic construct
  REGION 1..37: note = Description of Artificial Sequence: Synthetic polypeptide
GEQNPIYWAQYADWLFTTPLLLLDLALLVDADEGTCG

SEQ ID NO: 48 - protein, 38 aa, synthetic construct
  REGION 1..38: note = Description of Artificial Sequence: Synthetic polypeptide
GGEQNPIYWARYADWLFTTPLLLDLLALLVDADEGTCG

SEQ ID NO: 49 - protein, 38 aa, synthetic construct
  REGION 1..38: note = Description of Artificial Sequence: Synthetic polypeptide
GGEQNPIYWARYADWLFTTPLLLLLDALLVDADEGTCG

SEQ ID NO: 50 - protein, 38 aa, synthetic construct
  REGION 1..38: note = Description of Artificial Sequence: Synthetic polypeptide
GGEQNPIYWARYDAWLFTTPLLLLDLALLVDADEGTCG

SEQ ID NO: 51 - protein, 38 aa, synthetic construct
  REGION 1..38: note = Description of Artificial Sequence: Synthetic polypeptide
GGEQNPIYWARYAWDLFTTPLLLLDLALLVDADEGTCG

SEQ ID NO: 52 - protein, 36 aa, synthetic construct
  REGION 1..36: note = Description of Artificial Sequence: Synthetic polypeptide
AAEQNPIYWARYADWLFTTGLLLLDLALLVDADEGT

SEQ ID NO: 53 - protein, 37 aa, synthetic construct
  REGION 1..37: note = Description of Artificial Sequence: Synthetic polypeptide
DDDEDNPIYWARYADWLFTTPLLLLHGALLVDADECT

SEQ ID NO: 54 - protein, 36 aa, synthetic construct
  REGION 1..36: note = Description of Artificial Sequence: Synthetic polypeptide
DDDEDNPIYWARYADWLFTTPLLLLHGALLVDADET

SEQ ID NO: 55 - protein, 38 aa, synthetic construct
  REGION 1..38: note = Description of Artificial Sequence: Synthetic polypeptide
DDDEDNPIYWARYAHWLFTTPLLLLHGALLVDADEGCT

SEQ ID NO: 56 - protein, 37 aa, synthetic construct
  REGION 1..37: note = Description of Artificial Sequence: Synthetic polypeptide
CDDDEDNPIYWARYAHWLFTTPLLLLHGALLVDADET

SEQ ID NO: 57 - protein, 37 aa, synthetic construct
  REGION 1..37: note = Description of Artificial Sequence: Synthetic polypeptide
DDDEDNPIYWARYAHWLFTTPLLLLHGALLVDADEGT

SEQ ID NO: 58 - protein, 37 aa, synthetic construct
  REGION 1..37: note = Description of Artificial Sequence: Synthetic polypeptide
DDDEDNPIYWARYAHWLFTTPLLLLHGALLVNADECT

SEQ ID NO: 59 - protein, 37 aa, synthetic construct
  REGION 1..37: note = Description of Artificial Sequence: Synthetic polypeptide
DDDEDNPIYWARYAHWLFTTPLLLLHGALLVNADEGT

SEQ ID NO: 60 - protein, 37 aa, synthetic construct
  REGION 1..37: note = Description of Artificial Sequence: Synthetic polypeptide
DDDEDNPIYWARYAHWLFTTPLLLLHGALLVNANECT

SEQ ID NO: 61 - protein, 37 aa, synthetic construct
  REGION 1..37: note = Description of Artificial Sequence: Synthetic polypeptide
DDDEDNPIYWARYAHWLFTTPLLLLHGALLVNANEGT

SEQ ID NO: 62 - protein, 34 aa, synthetic construct
  REGION 1..34: note = Description of Artificial Sequence: Synthetic polypeptide
AEQNPIYWARYADFLFTTPLLLLDLALLVDADET

SEQ ID NO: 63 - protein, 35 aa, synthetic construct
  REGION 1..35: note = Description of Artificial Sequence: Synthetic polypeptide
AEQNPIYFARYADWLFTTPLLLLDLALLVDADEGT

SEQ ID NO: 64 - protein, 34 aa, synthetic construct
  REGION 1..34: note = Description of Artificial Sequence: Synthetic polypeptide
AEQNPIYFARYADFLFTTPLLLLDLALLWDADET

SEQ ID NO: 65 - protein, 32 aa, synthetic construct
  REGION 1..32: note = Description of Artificial Sequence: Synthetic polypeptide
AKEDQNPYWARYADWLFTTPLLLLDLALLVDG

SEQ ID NO: 66 - protein, 32 aa, synthetic construct
  REGION 1..32: note = Description of Artificial Sequence: Synthetic polypeptide
ACEDQNPYWARYADWLFTTPLLLLDLALLVDG

SEQ ID NO: 67 - protein, 32 aa, synthetic construct
  REGION 1..32: note = Description of Artificial Sequence: Synthetic polypeptide
AEDQNPYWARYADWLFTTPLLLLDLALLVDCG

SEQ ID NO: 68 - protein, 32 aa, synthetic construct
  REGION 1..32: note = Description of Artificial Sequence: Synthetic polypeptide
AEDQNPYWARYADWLFTTPLLLLELALLVECG

SEQ ID NO: 69 - protein, 32 aa, synthetic construct
  REGION 1..32: note = Description of Artificial Sequence: Synthetic polypeptide
AKEDQNDPYWARYADWLFTTPLLLLDLALLVG

SEQ ID NO: 70 - protein, 30 aa, synthetic construct
  REGION 1..30: note = Description of Artificial Sequence: Synthetic polypeptide
AKEDQNPYWRAYADLFTPLTLLDLLALWDG

SEQ ID NO: 71 - protein, 31 aa, synthetic construct
  REGION 1..31: note = Description of Artificial Sequence: Synthetic polypeptide
AEDQNPYWARYADWLFTTPLLLLELALLVCG

SEQ ID NO: 72 - protein, 30 aa, synthetic construct
  REGION 1..30: note = Description of Artificial Sequence: Synthetic polypeptide
ACEDQNPYWRAYADLFTPLTLLDLLALWDG

SEQ ID NO: 73 - protein, 27 aa, synthetic construct
  REGION 1..27: note = Description of Artificial Sequence: Synthetic peptide
ACDDQNPWRAYLDLLFPTDTLLLDLLW

SEQ ID NO: 74 - protein, 28 aa, synthetic construct
  REGION 1..28: note = Description of Artificial Sequence: Synthetic peptide
AKDDQNPWRAYLDLLFPTDTLLLDLLWC

SEQ ID NO: 75 - protein, 27 aa, synthetic construct
  REGION 1..27: note = Description of Artificial Sequence: Synthetic peptide
ACEEQNPWRAYLELLFPTETLLLELLW

SEQ ID NO: 76 - protein, 25 aa, synthetic construct
  REGION 1..25: note = Description of Artificial Sequence: Synthetic peptide
ACDDQNPWARYLDWLFPTDTLLLDL

SEQ ID NO: 77 - protein, 24 aa, synthetic construct
  REGION 1..24: note = Description of Artificial Sequence: Synthetic peptide
CDNNNPWRAYLDLLFPTDTLLLDW

SEQ ID NO: 78 - protein, 24 aa, synthetic construct
  REGION 1..24: note = Description of Artificial Sequence: Synthetic peptide
CEEQQPWAQYLELLFPTETLLLEW

SEQ ID NO: 79 - protein, 24 aa, synthetic construct
  REGION 1..24: note = Description of Artificial Sequence: Synthetic peptide
CEEQQPWRAYLELLFPTETLLLEW

SEQ ID NO: 80 - protein, 37 aa, synthetic construct
  REGION 1..37: note = Description of Artificial Sequence: Synthetic polypeptide
CDDDDDNPNYWARYANWLFTTPLLLLNGALLVEAEET

SEQ ID NO: 81 - protein, 36 aa, synthetic construct
  REGION 1..36: note = Description of Artificial Sequence: Synthetic polypeptide
CDDDDDNPNYWARYAPWLFTTPLLLLPGALLVEAEE

SEQ ID NO: 82 - protein, 34 aa, synthetic construct
  REGION 1..34: note = Description of Artificial Sequence: Synthetic polypeptide
TEDADVLLALDLLLLPTTFLWDAYRAWYPNQECA

SEQ ID NO: 83 - protein, 20 aa, synthetic construct
  REGION 1..20: note = Description of Artificial Sequence: Synthetic peptide
AEQNPIYWARYADWLFTTPL

SEQ ID NO: 84 - protein, 21 aa, synthetic construct
  REGION 1..21: note = Description of Artificial Sequence: Synthetic peptide
AEQNPIYWARYADWLFTTPCL

SEQ ID NO: 85 - protein, 21 aa, synthetic construct
  REGION 1..21: note = Description of Artificial Sequence: Synthetic peptide
ACEQNPIYWARYADWLFTTPL

SEQ ID NO: 86 - protein, 20 aa, synthetic construct
  REGION 1..20: note = Description of Artificial Sequence: Synthetic peptide
AEQNPIYFARYADWLFTTPL

SEQ ID NO: 87 - protein, 22 aa, synthetic construct
  REGION 1..22: note = Description of Artificial Sequence: Synthetic peptide
AEQNPIYFARYADLLFPTTLAW

SEQ ID NO: 88 - protein, 22 aa, synthetic construct
  REGION 1..22: note = Description of Artificial Sequence: Synthetic peptide
AEQNPIYWARYADLLFPTTLAF

SEQ ID NO: 89 - protein, 22 aa, synthetic construct
  REGION 1..22: note = Description of Artificial Sequence: Synthetic peptide
AEQNPIYWARYADLLFPTTLAW

SEQ ID NO: 90 - protein, 21 aa, synthetic construct
  REGION 1..21: note = Description of Artificial Sequence: Synthetic peptide
KEDQNPWARYADLLFPTTLAW

SEQ ID NO: 91 - protein, 20 aa, synthetic construct
  REGION 1..20: note = Description of Artificial Sequence: Synthetic peptide
KEDQNPWARYADLLFPTTLW

SEQ ID NO: 92 - protein, 22 aa, synthetic construct
  REGION 1..22: note = Description of Artificial Sequence: Synthetic peptide
ACEDQNPWARYADLLFPTTLAW

SEQ ID NO: 93 - protein, 24 aa, synthetic construct
  REGION 1..24: note = Description of Artificial Sequence: Synthetic peptide
ACEDQNPWARYADWLFPTTLLLLD

SEQ ID NO: 94 - protein, 22 aa, synthetic construct
  REGION 1..22: note = Description of Artificial Sequence: Synthetic peptide
ACEEQNPWARYAELLFPTTLAW

SEQ ID NO: 95 - protein, 24 aa, synthetic construct
  REGION 1..24: note = Description of Artificial Sequence: Synthetic peptide
ACEEQNPWARYAEWLFPTTLLLLE

SEQ ID NO: 96 - protein, 25 aa, synthetic construct
  REGION 1..25: note = Description of Artificial Sequence: Synthetic peptide
ACEEQNPWARYLEWLFPTETLLLEL

SEQ ID NO: 97 - protein, 24 aa, synthetic construct
  REGION 1..24: note = Description of Artificial Sequence: Synthetic peptide
ACEEQNPQAEYAEWLFPTTLLLLE

SEQ ID NO: 98 - protein, 21 aa, synthetic construct
  REGION 1..21: note = Description of Artificial Sequence: Synthetic peptide
  REGION 1..21: note = See specification as filed for detailed description of substitutions and preferred embodiments
WARYADWLFTTPLLLLDLALL

SEQ ID NO: 99 - protein, 21 aa, synthetic construct
  REGION 1..21: note = Description of Artificial Sequence: Synthetic peptide
  REGION 1..21: note = See specification as filed for detailed description of substitutions and preferred embodiments
YARYADWLFTTPLLLLDLALL

SEQ ID NO: 100 - protein, 21 aa, synthetic construct
  REGION 1..21: note = Description of Artificial Sequence: Synthetic peptide
  REGION 1..21: note = See specification as filed for detailed description of substitutions and preferred embodiments
WARYSDWLFTTPLLLYDLGLL

SEQ ID NO: 101 - protein, 21 aa, synthetic construct
  REGION 1..21: note = Description of Artificial Sequence: Synthetic peptide
  REGION 1..21: note = See specification as filed for detailed description of substitutions and preferred embodiments
WARYTDWFTTPLLLYDLALLA

SEQ ID NO: 102 - protein, 21 aa, synthetic construct
  REGION 1..21: note = Description of Artificial Sequence: Synthetic peptide
  REGION 1..21: note = See specification as filed for detailed description of substitutions and preferred embodiments
WARYTDWLFTTPLLLYDLGLL

SEQ ID NO: 103 - protein, 21 aa, synthetic construct
  REGION 1..21: note = Description of Artificial Sequence: Synthetic peptide
  REGION 1..21: note = See specification as filed for detailed description of substitutions and preferred embodiments
WARYADWLFTTPLLLLDLSLL

SEQ ID NO: 104 - protein, 21 aa, synthetic construct
  REGION 1..21: note = Description of Artificial Sequence: Synthetic peptide
  REGION 1..21: note = See specification as filed for detailed description of substitutions and preferred embodiments
LLALDLLLLPTTFLWDAYRAW

SEQ ID NO: 105 - protein, 21 aa, synthetic construct
  REGION 1..21: note = Description of Artificial Sequence: Synthetic peptide
  REGION 1..21: note = See specification as filed for detailed description of substitutions and preferred embodiments
LLALDLLLLPTTFLWDAYRAY

SEQ ID NO: 106 - protein, 21 aa, synthetic construct
  REGION 1..21: note = Description of Artificial Sequence: Synthetic peptide
  REGION 1..21: note = See specification as filed for detailed description of substitutions and preferred embodiments
LLGLDYLLLPTTFLWDSYRAW

SEQ ID NO: 107 - protein, 21 aa, synthetic construct
  REGION 1..21: note = Description of Artificial Sequence: Synthetic peptide
  REGION 1..21: note = See specification as filed for detailed description of substitutions and preferred embodiments
ALLALDYLLLPTTFWDTYRAW

SEQ ID NO: 108 - protein, 21 aa, synthetic construct
  REGION 1..21: note = Description of Artificial Sequence: Synthetic peptide
  REGION 1..21: note = See specification as filed for detailed description of substitutions and preferred embodiments
LLGLDYLLLPTTFLWDTYRAW

SEQ ID NO: 109 - protein, 21 aa, synthetic construct
  REGION 1..21: note = Description of Artificial Sequence: Synthetic peptide
  REGION 1..21: note = See specification as filed for detailed description of substitutions and preferred embodiments
LLSLDLLLLPTTFLWDAYRAW

SEQ ID NO: 110 - protein, 26 aa, synthetic construct
  REGION 1..26: note = Description of Artificial Sequence: Synthetic peptide
  REGION 1..26: note = See specification as filed for detailed description of substitutions and preferred embodiments
GLAGLLGLEGLLGLPLGLLEGLWLGL

SEQ ID NO: 111 - protein, 26 aa, synthetic construct
  REGION 1..26: note = Description of Artificial Sequence: Synthetic peptide
  REGION 1..26: note = See specification as filed for detailed description of substitutions and preferred embodiments
LGLWLGELLGLPLGLLGELGLLGALG

SEQ ID NO: 112 - protein, 20 aa, synthetic construct
  REGION 1..20: note = Description of Artificial Sequence: Synthetic peptide
  REGION 1..20: note = See specification as filed for detailed description of substitutions and preferred embodiments
WRAYLDLLFPTDTLLLDLLW

SEQ ID NO: 113 - protein, 20 aa, synthetic construct
  REGION 1..20: note = Description of Artificial Sequence: Synthetic peptide
  REGION 1..20: note = See specification as filed for detailed description of substitutions and preferred embodiments
WLLDLLLTDTPFLLDLYARW

SEQ ID NO: 114 - protein, 18 aa, synthetic construct
  REGION 1..18: note = Description of Artificial Sequence: Synthetic peptide
  REGION 1..18: note = See specification as filed for detailed description of substitutions and preferred embodiments
WARYLEWLFPTETLLLEL

SEQ ID NO: 115 - protein, 18 aa, synthetic construct
  REGION 1..18: note = Description of Artificial Sequence: Synthetic peptide
  REGION 1..18: note = See specification as filed for detailed description of substitutions and preferred embodiments
WAQYLELLFPTETLLLEW

SEQ ID NO: 116 - protein, 18 aa, synthetic construct
  REGION 1..18: note = Description of Artificial Sequence: Synthetic peptide
  REGION 1..18: note = See specification as filed for detailed description of substitutions and preferred embodiments
LELLLTETPFLWELYRAW

SEQ ID NO: 117 - protein, 18 aa, synthetic construct
  REGION 1..18: note = Description of Artificial Sequence: Synthetic peptide
  REGION 1..18: note = See specification as filed for detailed description of substitutions and preferred embodiments
WELLLTETPFLLELYQAW

SEQ ID NO: 118 - protein, 17 aa, synthetic construct
  REGION 1..17: note = Description of Artificial Sequence: Synthetic peptide
  REGION 1..17: note = See specification as filed for detailed description of substitutions and preferred embodiments
WLFTTPLLLLNGALLVE

SEQ ID NO: 119 - protein, 17 aa, synthetic construct
  REGION 1..17: note = Description of Artificial Sequence: Synthetic peptide
  REGION 1..17: note = See specification as filed for detailed description of substitutions and preferred embodiments
WLFTTPLLLLPGALLVE

SEQ ID NO: 120 - protein, 15 aa, synthetic construct
  REGION 1..15: note = Description of Artificial Sequence: Synthetic peptide
  REGION 1..15: note = See specification as filed for detailed description of substitutions and preferred embodiments
WARYADLLFPTTLAW

SEQ ID NO: 121 - protein, 17 aa, synthetic construct
  REGION 1..17: note = Description of Artificial Sequence: Synthetic peptide
  REGION 1..17: note = See specification as filed for detailed description of substitutions and preferred embodiments
EVLLAGNLLLLPTTFLW

SEQ ID NO: 122 - protein, 17 aa, synthetic construct
  REGION 1..17: note = Description of Artificial Sequence: Synthetic peptide
  REGION 1..17: note = See specification as filed for detailed description of substitutions and preferred embodiments
EVLLAGPLLLLPTTFLW

SEQ ID NO: 123 - protein, 15 aa, synthetic construct
  REGION 1..15: note = Description of Artificial Sequence: Synthetic peptide
  REGION 1..15: note = See specification as filed for detailed description of substitutions and preferred embodiments
WALTTPFLLDAYRAW

SEQ ID NO: 124 - protein, 24 aa, synthetic construct
  REGION 1..24: note = Description of Artificial Sequence: Synthetic peptide
  REGION 1..24: note = See specification as filed for detailed description of substitutions and preferred embodiments
NLEGFFATLGGEIALWSLVVLAIE

SEQ ID NO: 125 - protein, 22 aa, synthetic construct
  REGION 1..22: note = Description of Artificial Sequence: Synthetic peptide
  REGION 1..22: note = See specification as filed for detailed description of substitutions and preferred embodiments
EGFFATLGGEIALWSDVVLAIE

SEQ ID NO: 126 - protein, 22 aa, synthetic construct
  REGION 1..22: note = Description of Artificial Sequence: Synthetic peptide
  REGION 1..22: note = See specification as filed for detailed description of substitutions and preferred embodiments
EGFFATLGGEIPLWSDVVLAIE

SEQ ID NO: 127 - protein, 24 aa, synthetic construct
  REGION 1..24: note = Description of Artificial Sequence: Synthetic peptide
  REGION 1..24: note = See specification as filed for detailed description of substitutions and preferred embodiments
EIALVVLSWLAIEGGLTAFFGELN

SEQ ID NO: 128 - protein, 22 aa, synthetic construct
  REGION 1..22: note = Description of Artificial Sequence: Synthetic peptide
  REGION 1..22: note = See specification as filed for detailed description of substitutions and preferred embodiments
EIALVVDSWLAIEGGLTAFFGE

SEQ ID NO: 129 - protein, 22 aa, synthetic construct
  REGION 1..22: note = Description of Artificial Sequence: Synthetic peptide
  REGION 1..22: note = See specification as filed for detailed description of substitutions and preferred embodiments
EIALVVDSWLPIEGGLTAFFGE

SEQ ID NO: 130 - protein, 21 aa, synthetic construct
  REGION 1..21: note = Description of Artificial Sequence: Synthetic peptide
  REGION 1..21: note = See specification as filed for detailed description of substitutions and preferred embodiments
ILDLVFGLLFAVTSVDFLVQW

SEQ ID NO: 131 - protein, 21 aa, synthetic construct
  REGION 1..21: note = Description of Artificial Sequence: Synthetic peptide
  REGION 1..21: note = See specification as filed for detailed description of substitutions and preferred embodiments
WQVLFDVSTVAFLLGFVLDLI

SEQ ID NO: 132 - protein, 34 aa, synthetic construct
  REGION 1..34: note = Description of Artificial Sequence: Synthetic polypeptide
AEQNPIYWARYADWLFTTPLLLLDLALLVDADET

SEQ ID NO: 133 - protein, 34 aa, synthetic construct
  REGION 1..34: note = Description of Artificial Sequence: Synthetic polypeptide
AEQNPIYWARYAEWLFTTPLLLLELALLVEAEET

SEQ ID NO: 134 - protein, 33 aa, synthetic construct
  REGION 1..33: note = Description of Artificial Sequence: Synthetic polypeptide
ADDQNPWRAYLDLLFPDTTDLLLLDLLWDADET

SEQ ID NO: 135 - protein, 33 aa, synthetic construct
  REGION 1..33: note = Description of Artificial Sequence: Synthetic polypeptide
AEEQNPWRAYLELLFPETTELLLLELLWEAEET

SEQ ID NO: 136 - protein, 34 aa, synthetic construct
  REGION 1..34: note = Description of Artificial Sequence: Synthetic polypeptide
  MOD_RES 13: note = gamma-carboxyglutamic acid
AEQNPIYWARYAXWLFTTPLLLLDLALLVDADET

SEQ ID NO: 137 - protein, 34 aa, synthetic construct
  REGION 1..34: note = Description of Artificial Sequence: Synthetic polypeptide
  MOD_RES 24: note = gamma-carboxyglutamic acid
AEQNPIYWARYADWLFTTPLLLLXLALLVDADET

SEQ ID NO: 138 - protein, 34 aa, synthetic construct
  REGION 1..34: note = Description of Artificial Sequence: Synthetic polypeptide
  MOD_RES 13: note = gamma-carboxyglutamic acid
  MOD_RES 24: note = gamma-carboxyglutamic acid
AEQNPIYWARYAXWLFTTPLLLLXLALLVDADET

SEQ ID NO: 139 - protein, 34 aa, synthetic construct; identical to SEQ ID NO: 136
  REGION 1..34: note = Description of Artificial Sequence: Synthetic polypeptide
  MOD_RES 13: note = Aad

SEQ ID NO: 140 - protein, 34 aa, synthetic construct; identical to SEQ ID NO: 137
  REGION 1..34: note = Description of Artificial Sequence: Synthetic polypeptide
  MOD_RES 24: note = Aad

SEQ ID NO: 141 - protein, 34 aa, synthetic construct; identical to SEQ ID NO: 138
  REGION 1..34: note = Description of Artificial Sequence: Synthetic polypeptide
  MOD_RES 13: note = Aad
  MOD_RES 24: note = Aad

SEQ ID NO: 142 - protein, 34 aa, synthetic construct; identical to SEQ ID NO: 138
  REGION 1..34: note = Description of Artificial Sequence: Synthetic polypeptide
  MOD_RES 13: note = gamma-carboxyglutamic acid
  MOD_RES 24: note = Aad

SEQ ID NO: 143 - protein, 34 aa, synthetic construct; identical to SEQ ID NO: 138
  REGION 1..34: note = Description of Artificial Sequence: Synthetic polypeptide
  MOD_RES 13: note = Aad
  MOD_RES 24: note = gamma-carboxyglutamic acid

SEQ ID NO: 144 - protein, 36 aa, synthetic construct; identical to SEQ ID NO: 22
  REGION 1..36: note = Description of Artificial Sequence: Synthetic polypeptide

SEQ ID NO: 145 - protein, 36 aa, synthetic construct; identical to SEQ ID NO: 22
  REGION 1..36: note = Description of Artificial Sequence: Synthetic polypeptide

SEQ ID NO: 146 - protein, 36 aa, synthetic construct
  REGION 1..36: note = Description of Artificial Sequence: Synthetic polypeptide
AAEQNPIYWARYADWLFTTPLLLLDLALLVDADEGT

SEQ ID NO: 147 - protein, 35 aa, synthetic construct; identical to SEQ ID NO: 23
  REGION 1..35: note = Description of Artificial Sequence: Synthetic polypeptide

SEQ ID NO: 148 - protein, 35 aa, synthetic construct
  REGION 1..35: note = Description of Artificial Sequence: Synthetic polypeptide
AEQNPIYWARYANWLFTTPLLLLNLALLVDADEGT

SEQ ID NO: 149 - protein, 35 aa, synthetic construct
  REGION 1..35: note = Description of Artificial Sequence: Synthetic polypeptide
AEQNPIYWARYAKWLFTTPLLLLKLALLVDADEGT

SEQ ID NO: 150 - protein, 36 aa, synthetic construct; identical to SEQ ID NO: 39
  REGION 1..36: note = Description of Artificial Sequence: Synthetic polypeptide

SEQ ID NO: 151 - protein, 36 aa, synthetic construct; identical to SEQ ID NO: 40
  REGION 1..36: note = Description of Artificial Sequence: Synthetic polypeptide

SEQ ID NO: 152 - protein, 36 aa, synthetic construct; identical to SEQ ID NO: 42
  REGION 1..36: note = Description of Artificial Sequence: Synthetic polypeptide

SEQ ID NO: 153 - protein, 36 aa, synthetic construct; identical to SEQ ID NO: 43
  REGION 1..36: note = Description of Artificial Sequence: Synthetic polypeptide

SEQ ID NO: 154 - protein, 36 aa, synthetic construct; identical to SEQ ID NO: 44
  REGION 1..36: note = Description of Artificial Sequence: Synthetic polypeptide

SEQ ID NO: 155 - protein, 36 aa, synthetic construct; identical to SEQ ID NO: 45
  REGION 1..36: note = Description of Artificial Sequence: Synthetic polypeptide

SEQ ID NO: 156 - protein, 39 aa, synthetic construct; identical to SEQ ID NO: 46
  REGION 1..39: note = Description of Artificial Sequence: Synthetic polypeptide

SEQ ID NO: 157 - protein, 34 aa, synthetic construct
  REGION 1..34: note = Description of Artificial Sequence: Synthetic polypeptide
GEQNPIYWAQYADWLFTTPLLLLDLALLVDADEG

SEQ ID NO: 158 - protein, 35 aa, synthetic construct
  REGION 1..35: note = Description of Artificial Sequence: Synthetic polypeptide
GGEQNPIYWARYADWLFTTPLLLDLLALLVDADEG

SEQ ID NO: 159 - protein, 35 aa, synthetic construct
  REGION 1..35: note = Description of Artificial Sequence: Synthetic polypeptide
GGEQNPIYWARYADWLFTTPLLLLLDALLVDADEG

SEQ ID NO: 160 - protein, 36 aa, synthetic construct
  REGION 1..36: note = Description of Artificial Sequence: Synthetic polypeptide
GGEQNPIYWARYDAWLFTTPLLLLDLALLVDADEGT

SEQ ID NO: 161 - protein, 35 aa, synthetic construct
  REGION 1..35: note = Description of Artificial Sequence: Synthetic polypeptide
GGEQNPIYWARYAWDLFTTPLLLLDLALLVDADEG

SEQ ID NO: 162 - protein, 36 aa, synthetic construct; identical to SEQ ID NO: 52
  REGION 1..36: note = Description of Artificial Sequence: Synthetic polypeptide

SEQ ID NO: 163 - protein, 34 aa, synthetic construct
  REGION 1..34: note = Description of Artificial Sequence: Synthetic polypeptide
DDDEDNPIYWARYADWLFTTPLLLLHGALLVDAD

SEQ ID NO: 164 - protein, 35 aa, synthetic construct
  REGION 1..35: note = Description of Artificial Sequence: Synthetic polypeptide
DDDEDNPIYWARYAHWLFTTPLLLLHGALLVDADE

SEQ ID NO: 165 - protein, 33 aa, synthetic construct
  REGION 1..33: note = Description of Artificial Sequence: Synthetic polypeptide
CEQNPIYWARYADWHFTTPLLLLDLALLVDADE

SEQ ID NO: 166 - protein, 34 aa, synthetic construct; identical to SEQ ID NO: 62
  REGION 1..34: note = Description of Artificial Sequence: Synthetic polypeptide

SEQ ID NO: 167 - protein, 33 aa, synthetic construct
  REGION 1..33: note = Description of Artificial Sequence: Synthetic polypeptide
AEQNPIYFARYADWLFTTPLLLLDLALLVDADE

SEQ ID NO: 168 - protein, 34 aa, synthetic construct; identical to SEQ ID NO: 64
  REGION 1..34: note = Description of Artificial Sequence: Synthetic polypeptide

SEQ ID NO: 169 - protein, 32 aa, synthetic construct
  REGION 1..32: note = Description of Artificial Sequence: Synthetic polypeptide
ADNNPWIYARYADLTTFPLLLLDLALLVDFDD

SEQ ID NO: 170 - protein, 32 aa, synthetic construct
  REGION 1..32: note = Description of Artificial Sequence: Synthetic polypeptide
ADNNPFIYARYADLTTWPLLLLDLALLVDFDD

SEQ ID NO: 171 - protein, 32 aa, synthetic construct
  REGION 1..32: note = Description of Artificial Sequence: Synthetic polypeptide
ADNNPFIYARYADLTTFPLLLLDLALLVDWDD

SEQ ID NO: 172 - protein, 32 aa, synthetic construct
  REGION 1..32: note = Description of Artificial Sequence: Synthetic polypeptide
ADNNPFPYARYADLTTWILLLLDLALLVDFDD

SEQ ID NO: 173 - protein, 32 aa, synthetic construct
  REGION 1..32: note = Description of Artificial Sequence: Synthetic polypeptide
ADNNPFIYAYRADLTTFPLLLLDLALLVDWDD

SEQ ID NO: 174 - protein, 32 aa, synthetic construct
  REGION 1..32: note = Description of Artificial Sequence: Synthetic polypeptide
ADNNPFIYATYADLRTFPLLLLDLALLVDWDD

SEQ ID NO: 175 - protein, 33 aa, synthetic construct
  REGION 1..33: note = Description of Artificial Sequence: Synthetic polypeptide
TEDADVLLALDLLLLPTTFLWDAYRAWYPNQEA

SEQ ID NO: 176 - protein, 30 aa, synthetic construct
  REGION 1..30: note = Description of Artificial Sequence: Synthetic polypeptide
AEDQNPYWARYADWLFTTPLLLLDLALLVD

SEQ ID NO: 177 - protein, 30 aa, synthetic construct
  REGION 1..30: note = Description of Artificial Sequence: Synthetic polypeptide
AEDQNPYWARYADWLFTTPLLLLELALLVE

SEQ ID NO: 178 - protein, 28 aa, synthetic construct
  REGION 1..28: note = Description of Artificial Sequence: Synthetic peptide
AEDQNPYWRAYADLFTPLTLLDLLALWD

SEQ ID NO: 179 - protein, 26 aa, synthetic construct
  REGION 1..26: note = Description of Artificial Sequence: Synthetic peptide
ADDQNPWRAYLDLLFPTDTLLLDLLW

SEQ ID NO: 180 - protein, 30 aa, synthetic construct
  REGION 1..30: note = Description of Artificial Sequence: Synthetic polypeptide
ADDQNPWRAYLDLLFPTDTLLLDLLWDADE

SEQ ID NO: 181 - protein, 26 aa, synthetic construct
  REGION 1..26: note = Description of Artificial Sequence: Synthetic peptide
  MOD_RES 12: note = gamma-carboxyglutamic acid
ADDQNPWRAYLXLLFPTDTLLLDLLW

SEQ ID NO: 182 - protein, 26 aa, synthetic construct
  REGION 1..26: note = Description of Artificial Sequence: Synthetic peptide
  MOD_RES 18: note = gamma-carboxyglutamic acid
ADDQNPWRAYLDLLFPTXTLLLDLLW

SEQ ID NO: 183 - protein, 26 aa, synthetic construct
  REGION 1..26: note = Description of Artificial Sequence: Synthetic peptide
  MOD_RES 23: note = gamma-carboxyglutamic acid
ADDQNPWRAYLDLLFPTDTLLLXLLW

SEQ ID NO: 184 - protein, 26 aa, synthetic construct
  REGION 1..26: note = Description of Artificial Sequence: Synthetic peptide
  MOD_RES 12: note = gamma-carboxyglutamic acid
  MOD_RES 18: note = gamma-carboxyglutamic acid
ADDQNPWRAYLXLLFPTXTLLLDLLW

SEQ ID NO: 185 - protein, 26 aa, synthetic construct
  REGION 1..26: note = Description of Artificial Sequence: Synthetic peptide
  MOD_RES 12: note = gamma-carboxyglutamic acid
  MOD_RES 23: note = gamma-carboxyglutamic acid
ADDQNPWRAYLXLLFPTDTLLLXLLW

SEQ ID NO: 186 - protein, 26 aa, synthetic construct
  REGION 1..26: note = Description of Artificial Sequence: Synthetic peptide
  MOD_RES 18: note = Gla
  MOD_RES 23: note = Gla
ADDQNPWRAYLDLLFPTXTLLLXLLW

SEQ ID NO: 187 - protein, 26 aa, synthetic construct
  REGION 1..26: note = Description of Artificial Sequence: Synthetic peptide
  MOD_RES 12: note = gamma-carboxyglutamic acid
  MOD_RES 18: note = gamma-carboxyglutamic acid
  MOD_RES 23: note = gamma-carboxyglutamic acid
ADDQNPWRAYLXLLFPTXTLLLXLLW

SEQ ID NO: 188 - protein, 26 aa, synthetic construct
  REGION 1..26: note = Description of Artificial Sequence: Synthetic peptide
  MOD_RES 12: note = Aad
ADDQNPWRAYLXLLFPTDTLLLDLLW

SEQ ID NO: 189 - protein, 26 aa, synthetic construct
  REGION 1..26: note = Description of Artificial Sequence: Synthetic peptide
  MOD_RES 18: note = Aad
ADDQNPWRAYLDLLFPTXTLLLDLLW

SEQ ID NO: 190 - protein, 26 aa, synthetic construct
  REGION 1..26: note = Description of Artificial Sequence: Synthetic peptide
  MOD_RES 23: note = Aad
ADDQNPWRAYLDLLFPTDTLLLXLLW

SEQ ID NO: 191 - protein, 26 aa, synthetic construct
  REGION 1..26: note = Description of Artificial Sequence: Synthetic peptide
  MOD_RES 12: note = Aad
  MOD_RES 18: note = Aad
ADDQNPWRAYLXLLFPTXTLLLDLLW

SEQ ID NO: 192 - protein, 26 aa, synthetic construct
  REGION 1..26: note = Description of Artificial Sequence: Synthetic peptide
  MOD_RES 18: note = Aad
  MOD_RES 23: note = Aad
ADDQNPWRAYLDLLFPTXTLLLXLLW

SEQ ID NO: 193 - protein, 26 aa, synthetic construct
  REGION 1..26: note = Description of Artificial Sequence: Synthetic peptide
  MOD_RES 12: note = Aad
  MOD_RES 18: note = Aad
  MOD_RES 23: note = Aad
ADDQNPWRAYLXLLFPTXTLLLXLLW

SEQ ID NO: 194 - protein, 26 aa, synthetic construct
  REGION 1..26: note = Description of Artificial Sequence: Synthetic peptide
  MOD_RES 12: note = gamma-carboxyglutamic acid
  MOD_RES 18: note = Aad
ADDQNPWRAYLXLLFPTXTLLLDLLW

SEQ ID NO: 195 - protein, 26 aa, synthetic construct
  REGION 1..26: note = Description of Artificial Sequence: Synthetic peptide
  MOD_RES 12: note = gamma-carboxyglutamic acid
  MOD_RES 23: note = Aad
ADDQNPWRAYLXLLFPTDTLLLXLLW

SEQ ID NO: 196 - protein, 26 aa, synthetic construct
  REGION 1..26: note = Description of Artificial Sequence: Synthetic peptide
  MOD_RES 12: note = gamma-carboxyglutamic acid
  MOD_RES 18: note = gamma-carboxyglutamic acid
  MOD_RES 23: note = Aad
ADDQNPWRAYLXLLFPTXTLLLXLLW

SEQ ID NO: 197 - protein, 26 aa, synthetic construct
  REGION 1..26: note = Description of Artificial Sequence: Synthetic peptide
  MOD_RES 12: note = Aad
  MOD_RES 18: note = gamma-carboxyglutamic acid
ADDQNPWRAYLXLLFPTXTLLLDLLW

SEQ ID NO: 198 - protein, 26 aa, synthetic construct
  REGION 1..26: note = Description of Artificial Sequence: Synthetic peptide
  MOD_RES 12: note = Aad
  MOD_RES 23: note = gamma-carboxyglutamic acid
ADDQNPWRAYLXLLFPTDTLLLXLLW

SEQ ID NO: 199 - protein, 26 aa, synthetic construct
  REGION 1..26: note = Description of Artificial Sequence: Synthetic peptide
  MOD_RES 12: note = gamma-carboxyglutamic acid
  MOD_RES 18: note = Aad
  MOD_RES 23: note = gamma-carboxyglutamic acid
ADDQNPWRAYLXLLFPTXTLLLXLLW

SEQ ID NO: 200 - protein, 29 aa, synthetic construct
  REGION 1..29: note = Description of Artificial Sequence: Synthetic peptide
GEEQNPWLGAYLDLLFPLELLGLLELGLW

SEQ ID NO: 201 - protein, 28 aa, synthetic construct
  REGION 1..28: note = Description of Artificial Sequence: Synthetic peptide
ADDDDDDPWQAYLDLLFPTDTLLLDLLW

SEQ ID NO: 202 - protein, 26 aa, synthetic construct
  REGION 1..26: note = Description of Artificial Sequence: Synthetic peptide
AEEQNPWRAYLELLFPTETLLLELLW

SEQ ID NO: 203 - protein, 24 aa, synthetic construct
  REGION 1..24: note = Description of Artificial Sequence: Synthetic peptide
ADDQNPWARYLDWLFPTDTLLLDL

SEQ ID NO: 204 - protein, 23 aa, synthetic construct
  REGION 1..23: note = Description of Artificial Sequence: Synthetic peptide
DNNNPWRAYLDLLFPTDTLLLDW

SEQ ID NO: 205 - protein, 24 aa, synthetic construct
  REGION 1..24: note = Description of Artificial Sequence: Synthetic peptide
AEEQNPWARYLEWLFPTETLLLEL

SEQ ID NO: 206 - protein, 25 aa, synthetic construct
  REGION 1..25: note = Description of Artificial Sequence: Synthetic peptide
DDDDDDPWQAYLDLFPTDTLALDLW

SEQ ID NO: 207 - protein, 23 aa, synthetic construct
  REGION 1..23: note = Description of Artificial Sequence: Synthetic peptide
EEQQPWAQYLELLFPTETLLLEW

SEQ ID NO: 208 - protein, 23 aa, synthetic construct
  REGION 1..23: note = Description of Artificial Sequence: Synthetic peptide
EEQQPWRAYLELLFPTETLLLEW

SEQ ID NO: 209 - protein, 23 aa, synthetic construct
  REGION 1..23: note = Description of Artificial Sequence: Synthetic peptide
AEDQNPWARYADWLFPTTLLLLD

SEQ ID NO: 210 - protein, 23 aa, synthetic construct
  REGION 1..23: note = Description of Artificial Sequence: Synthetic peptide
AEEQNPWARYAEWLFPTTLLLLE

SEQ ID NO: 211 - protein, 21 aa, synthetic construct
  REGION 1..21: note = Description of Artificial Sequence: Synthetic peptide
AEDQNPWARYADLLFPTTLAW

SEQ ID NO: 212 - protein, 21 aa, synthetic construct
  REGION 1..21: note = Description of Artificial Sequence: Synthetic peptide
AEEQNPWARYAELLFPTTLAW

SEQ ID NO: 213 - protein, 36 aa, synthetic construct
  REGION 1..36: note = Description of Artificial Sequence: Synthetic polypeptide
DDDDDNPNYWARYANWLFTTPLLLLNGALLVEAEET

SEQ ID NO: 214 - protein, 36 aa, synthetic construct
  REGION 1..36: note = Description of Artificial Sequence: Synthetic polypeptide
DDDDDNPNYWARYAPWLFTTPLLLLPGALLVEAEET

SEQ ID NO: 215 - protein, 34 aa, synthetic construct; identical to SEQ ID NO: 64
  REGION 1..34: note = Description of Artificial Sequence: Synthetic polypeptide

SEQ ID NO: 216 - protein, 34 aa, synthetic construct; identical to SEQ ID NO: 62
  REGION 1..34: note = Description of Artificial Sequence: Synthetic polypeptide

SEQ ID NO: 217 - protein, 20 aa, synthetic construct
  REGION 1..20: note = Description of Artificial Sequence: Synthetic peptide
AEQNPIYWARYADWLFTTPL

SEQ ID NO: 218 - protein, 22 aa, synthetic construct
  REGION 1..22: note = Description of Artificial Sequence: Synthetic peptide
AEQNPIYFARYADLLFPTTLAW

SEQ ID NO: 219 - protein, 22 aa, synthetic construct
  REGION 1..22: note = Description of Artificial Sequence: Synthetic peptide
AEQNPIYWARYADLLFPTTLAF

SEQ ID NO: 220 - protein, 22 aa, synthetic construct
  REGION 1..22: note = Description of Artificial Sequence: Synthetic peptide
AEQNPIYWARYADLLFPTTLAW

SEQ ID NO: 221 - protein, 20 aa, synthetic construct
  REGION 1..20: note = Description of Artificial Sequence: Synthetic peptide
AEQNPIYFARYADWLFTTPL

SEQ ID NO: 222 - protein, 20 aa, synthetic construct
  REGION 1..20: note = Description of Artificial Sequence: Synthetic peptide
EDQNPWARYADLLFPTTLAW

SEQ ID NO: 223 - protein, 34 aa, synthetic construct
  REGION 1..34: note = Description of Artificial Sequence: Synthetic polypeptide
GLAGLAGLLGLEGLLGLPLGLLEGLWLGLELEGN

SEQ ID NO: 224 - protein, 32 aa, synthetic construct
  REGION 1..32: note = Description of Artificial Sequence: Synthetic polypeptide
EQNPIYILDLVFGLLFAVTSVDFLVQWDDAGD

SEQ ID NO: 225 - protein, 24 aa, synthetic construct
  REGION 1..24: note = Description of Artificial Sequence: Synthetic peptide
NLEGFFATLGGEIALWSLVVLAIE

SEQ ID NO: 226 - protein, 24 aa, synthetic construct
  REGION 1..24: note = Description of Artificial Sequence: Synthetic peptide
NNEGFFATLGGEIALWSDVVLAIE

SEQ ID NO: 227 - protein, 25 aa, synthetic construct
  REGION 1..25: note = Description of Artificial Sequence: Synthetic peptide
DNNEGFFATLGGEIPLWSDVVLAIE

SEQ ID NO: 228 - protein, 8 aa, synthetic construct
  REGION 1..8: note = Description of Artificial Sequence: Synthetic peptide
WEWEWEWC

SEQ ID NO: 229 - protein, 10 aa, synthetic construct
  REGION 1..10: note = Description of Artificial Sequence: Synthetic peptide
WEWEWEWEWC

SEQ ID NO: 230 - protein, 12 aa, synthetic construct
  REGION 1..12: note = Description of Artificial Sequence: Synthetic peptide
WEWEWEWEWEWC

SEQ ID NO: 231 - protein, 10 aa, synthetic construct
  REGION 1..10: note = Description of Artificial Sequence: Synthetic peptide
LELELELEWC

SEQ ID NO: 232 - protein, 10 aa, synthetic construct
  REGION 1..10: note = Description of Artificial Sequence: Synthetic peptide
EEEEWWWWWC

SEQ ID NO: 233 - protein, 10 aa, synthetic construct
  REGION 1..10: note = Description of Artificial Sequence: Synthetic peptide
CWEWEWEWEW

SEQ ID NO: 234 - protein, 10 aa, synthetic construct
  REGION 1..10: note = Description of Artificial Sequence: Synthetic peptide
RRRRWWWWWC

SEQ ID NO: 235 - protein, 9 aa, synthetic construct
  REGION 1..9: note = Description of Artificial Sequence: Synthetic peptide
EEEWWWWWC

SEQ ID NO: 236 - protein, 9 aa, synthetic construct
  REGION 1..9: note = Description of Artificial Sequence: Synthetic peptide
EWEWWWWEC

SEQ ID NO: 237 - protein, 9 aa, synthetic construct
  REGION 1..9: note = Description of Artificial Sequence: Synthetic peptide
EWWEWWWEC

SEQ ID NO: 238 - protein, 9 aa, synthetic construct
  REGION 1..9: note = Description of Artificial Sequence: Synthetic peptide
EWWWEWWEC

SEQ ID NO: 239 - protein, 9 aa, synthetic construct
  REGION 1..9: note = Description of Artificial Sequence: Synthetic peptide
EWWWWEWEC

SEQ ID NO: 240 - protein, 9 aa, synthetic construct
  REGION 1..9: note = Description of Artificial Sequence: Synthetic peptide
EWWWWWEEC

SEQ ID NO: 241 - protein, 9 aa, synthetic construct
  REGION 1..9: note = Description of Artificial Sequence: Synthetic peptide
EWEEWWWWC

SEQ ID NO: 242 - protein, 9 aa, synthetic construct
  REGION 1..9: note = Description of Artificial Sequence: Synthetic peptide
EWWEEWWWC

SEQ ID NO: 243 - protein, 9 aa, synthetic construct
  REGION 1..9: note = Description of Artificial Sequence: Synthetic peptide
EWWWEEWWC

SEQ ID NO: 244 - protein, 9 aa, synthetic construct
  REGION 1..9: note = Description of Artificial Sequence: Synthetic peptide
EWWWWEEWC

SEQ ID NO: 245 - protein, 9 aa, synthetic construct
  REGION 1..9: note = Description of Artificial Sequence: Synthetic peptide
WEEEWWWWC

SEQ ID NO: 246 - protein, 9 aa, synthetic construct
  REGION 1..9: note = Description of Artificial Sequence: Synthetic peptide
WWEEEWWWC

SEQ ID NO: 247 - protein, 9 aa, synthetic construct
  REGION 1..9: note = Description of Artificial Sequence: Synthetic peptide
WWWEEEWWC

SEQ ID NO: 248 - protein, 9 aa, synthetic construct
  REGION 1..9: note = Description of Artificial Sequence: Synthetic peptide
WWWWEEEWC

SEQ ID NO: 249 - protein, 9 aa, synthetic construct
  REGION 1..9: note = Description of Artificial Sequence: Synthetic peptide
WEWEEWWWC

SEQ ID NO: 250 - protein, 9 aa, synthetic construct
  REGION 1..9: note = Description of Artificial Sequence: Synthetic peptide
WEWWEEWWC

SEQ ID NO: 251 - protein, 9 aa, synthetic construct
  REGION 1..9: note = Description of Artificial Sequence: Synthetic peptide
WEWWWEEWC

SEQ ID NO: 252 - protein, 9 aa, synthetic construct
  REGION 1..9: note = Description of Artificial Sequence: Synthetic peptide
WEWWWWEEC

SEQ ID NO: 253 - protein, 8 aa, synthetic construct
  REGION 1..8: note = Description of Artificial Sequence: Synthetic peptide
EEEWWWWW

SEQ ID NO: 254 - protein, 8 aa, synthetic construct
  REGION 1..8: note = Description of Artificial Sequence: Synthetic peptide
EWEWWWWE

SEQ ID NO: 255 - protein, 8 aa, synthetic construct
  REGION 1..8: note = Description of Artificial Sequence: Synthetic peptide
EWWEWWWE

SEQ ID NO: 256 - protein, 8 aa, synthetic construct
  REGION 1..8: note = Description of Artificial Sequence: Synthetic peptide
EWWWEWWE

SEQ ID NO: 257 - protein, 8 aa, synthetic construct
  REGION 1..8: note = Description of Artificial Sequence: Synthetic peptide
EWWWWEWE

SEQ ID NO: 258 - protein, 8 aa, synthetic construct
  REGION 1..8: note = Description of Artificial Sequence: Synthetic peptide
EWWWWWEE

SEQ ID NO: 259 - protein, 8 aa, synthetic construct
  REGION 1..8: note = Description of Artificial Sequence: Synthetic peptide
EWEEWWWW

SEQ ID NO: 260 - protein, 8 aa, synthetic construct
  REGION 1..8: note = Description of Artificial Sequence: Synthetic peptide
EWWEEWWW

SEQ ID NO: 261 - protein, 8 aa, synthetic construct
  REGION 1..8: note = Description of Artificial Sequence: Synthetic peptide
EWWWEEWW

SEQ ID NO: 262 - protein, 8 aa, synthetic construct
  REGION 1..8: note = Description of Artificial Sequence: Synthetic peptide
EWWWWEEW

SEQ ID NO: 263 - protein, 8 aa, synthetic construct
  REGION 1..8: note = Description of Artificial Sequence: Synthetic peptide
WEEEWWWW

SEQ ID NO: 264 - protein, 8 aa, synthetic construct
  REGION 1..8: note = Description of Artificial Sequence: Synthetic peptide
WWEEEWWW

SEQ ID NO: 265 - protein, 8 aa, synthetic construct
  REGION 1..8: note = Description of Artificial Sequence: Synthetic peptide
WWWEEEWW

SEQ ID NO: 266 - protein, 8 aa, synthetic construct
  REGION 1..8: note = Description of Artificial Sequence: Synthetic peptide
WWWWEEEW

SEQ ID NO: 267 - protein, 8 aa, synthetic construct
  REGION 1..8: note = Description of Artificial Sequence: Synthetic peptide
WEWEEWWW

SEQ ID NO: 268 - protein, 8 aa, synthetic construct
  REGION 1..8: note = Description of Artificial Sequence: Synthetic peptide
WEWWEEWW

SEQ ID NO: 269 - protein, 8 aa, synthetic construct
  REGION 1..8: note = Description of Artificial Sequence: Synthetic peptide
WEWWWEEW

SEQ ID NO: 270 - protein, 8 aa, synthetic construct
  REGION 1..8: note = Description of Artificial Sequence: Synthetic peptide
WEWWWWEE

SEQ ID NO: 271 - protein, 9 aa, synthetic construct
  REGION 1..9: note = Description of Artificial Sequence: Synthetic peptide
DDDWWWWWC

SEQ ID NO: 272 - protein, 9 aa, synthetic construct
  REGION 1..9: note = Description of Artificial Sequence: Synthetic peptide
DWDWWWWDC

SEQ ID NO: 273 - protein, 9 aa, synthetic construct
  REGION 1..9: note = Description of Artificial Sequence: Synthetic peptide
DWWDWWWDC

SEQ ID NO: 274 - protein, 9 aa, synthetic construct
  REGION 1..9: note = Description of Artificial Sequence: Synthetic peptide
DWWWDWWDC

SEQ ID NO: 275 - protein, 9 aa, synthetic construct
  REGION 1..9: note = Description of Artificial Sequence: Synthetic peptide
DWWWWDWDC

SEQ ID NO: 276 - protein, 9 aa, synthetic construct
  REGION 1..9: note = Description of Artificial Sequence: Synthetic peptide
DWWWWWDDC

SEQ ID NO: 277 - protein, 9 aa, synthetic construct
  REGION 1..9: note = Description of Artificial Sequence: Synthetic peptide
DWDDWWWWC

SEQ ID NO: 278 - protein, 9 aa, synthetic construct
  REGION 1..9: note = Description of Artificial Sequence: Synthetic peptide
DWWDDWWWC

SEQ ID NO: 279 - protein, 9 aa, synthetic construct
  REGION 1..9: note = Description of Artificial Sequence: Synthetic peptide
DWWWDDWWC

SEQ ID NO: 280 - protein, 9 aa, synthetic construct
  REGION 1..9: note = Description of Artificial Sequence: Synthetic peptide
DWWWWDDWC

SEQ ID NO: 281 - protein, 9 aa, synthetic construct
  REGION 1..9: note = Description of Artificial Sequence: Synthetic peptide
WDDDWWWWC

SEQ ID NO: 282 - protein, 9 aa, synthetic construct
  REGION 1..9: note = Description of Artificial Sequence: Synthetic peptide
WWDDDWWWC

SEQ ID NO: 283 - protein, 9 aa, synthetic construct
  REGION 1..9: note = Description of Artificial Sequence: Synthetic peptide
WWWDDDWWC

SEQ ID NO: 284 - protein, 9 aa, synthetic construct
  REGION 1..9: note = Description of Artificial Sequence: Synthetic peptide
WWWWDDDWC

SEQ ID NO: 285 - protein, 9 aa, synthetic construct
  REGION 1..9: note = Description of Artificial Sequence: Synthetic peptide
WDWDDWWWC

SEQ ID NO: 286 - protein, 9 aa, synthetic construct
  REGION 1..9: note = Description of Artificial Sequence: Synthetic peptide
WDWWDDWWC

SEQ ID NO: 287 - protein, 9 aa, synthetic construct
  REGION 1..9: note = Description of Artificial Sequence: Synthetic peptide
WDWWWDDWC

SEQ ID NO: 288 - protein, 9 aa, synthetic construct
  REGION 1..9: note = Description of Artificial Sequence: Synthetic peptide
WDWWWWDDC

SEQ ID NO: 289 - protein, 8 aa, synthetic construct
  REGION 1..8: note = Description of Artificial Sequence: Synthetic peptide
DDDWWWWW

SEQ ID NO: 290 - protein, 8 aa, synthetic construct
  REGION 1..8: note = Description of Artificial Sequence: Synthetic peptide
DWDWWWWD

SEQ ID NO: 291 - protein, 8 aa, synthetic construct
  REGION 1..8: note = Description of Artificial Sequence: Synthetic peptide
DWWDWWWD

SEQ ID NO: 292 - protein, 8 aa, synthetic construct
  REGION 1..8: note = Description of Artificial Sequence: Synthetic peptide
DWWWDWWD

SEQ ID NO: 293 - protein, 8 aa, synthetic construct
  REGION 1..8: note = Description of Artificial Sequence: Synthetic peptide
DWWWWDWD

SEQ ID NO: 294 - protein, 8 aa, synthetic construct
  REGION 1..8: note = Description of Artificial Sequence: Synthetic peptide
DWWWWWDD

SEQ ID NO: 295 - protein, 8 aa, synthetic construct
  REGION 1..8: note = Description of Artificial Sequence: Synthetic peptide
DWDDWWWW

SEQ ID NO: 296 - protein, 8 aa, synthetic construct
  REGION 1..8: note = Description of Artificial Sequence: Synthetic peptide
DWWDDWWW

SEQ ID NO: 297 - protein, 8 aa, synthetic construct
  REGION 1..8: note = Description of Artificial Sequence: Synthetic peptide
DWWWDDWW

SEQ ID NO: 298 - protein, 8 aa, synthetic construct
  REGION 1..8: note = Description of Artificial Sequence: Synthetic peptide
DWWWWDDW

SEQ ID NO: 299 - protein, 8 aa, synthetic construct
  REGION 1..8: note = Description of Artificial Sequence: Synthetic peptide
WDDDWWWW

SEQ ID NO: 300 - protein, 8 aa, synthetic construct
  REGION 1..8: note = Description of Artificial Sequence: Synthetic peptide
WWDDDWWW

SEQ ID NO: 301 - protein, 8 aa, synthetic construct
  REGION 1..8: note = Description of Artificial Sequence: Synthetic peptide
WWWDDDWW

SEQ ID NO: 302 - protein, 8 aa, synthetic construct
  REGION 1..8: note = Description of Artificial Sequence: Synthetic peptide
WWWWDDDW

SEQ ID NO: 303 - protein, 8 aa, synthetic construct
  REGION 1..8: note = Description of Artificial Sequence: Synthetic peptide
WDWDDWWW

SEQ ID NO: 304 - protein, 8 aa, synthetic construct
  REGION 1..8: note = Description of Artificial Sequence: Synthetic peptide
WDWWDDWW

SEQ ID NO: 305 - protein, 8 aa, synthetic construct
  REGION 1..8: note = Description of Artificial Sequence: Synthetic peptide
WDWWWDDW

SEQ ID NO: 306 - protein, 8 aa, synthetic construct
  REGION 1..8: note = Description of Artificial Sequence: Synthetic peptide
WDWWWWDD

SEQ ID NO: 307 - protein, 8 aa, synthetic construct
  REGION 1..8: note = Description of Artificial Sequence: Synthetic peptide
  MOD_RES 1..3: note = gamma-carboxyglutamic acid
XXXWWWWW

SEQ ID NO: 308 - protein, 8 aa, synthetic construct
  REGION 1..8: note = Description of Artificial Sequence: Synthetic peptide
  MOD_RES 1: note = gamma-carboxyglutamic acid
  MOD_RES 3: note = gamma-carboxyglutamic acid
  MOD_RES 8: note = gamma-carboxyglutamic acid
XWXWWWWX

SEQ ID NO: 309 - protein, 8 aa, synthetic construct
  REGION 1..8: note = Description of Artificial Sequence: Synthetic peptide
  MOD_RES 1: note = gamma-carboxyglutamic acid
  MOD_RES 4: note = gamma-carboxyglutamic acid
  MOD_RES 8: note = gamma-carboxyglutamic acid
XWWXWWWX

SEQ ID NO: 310 - protein, 8 aa, synthetic construct
  REGION 1..8: note = Description of Artificial Sequence: Synthetic peptide
  MOD_RES 1: note = gamma-carboxyglutamic acid
  MOD_RES 5: note = gamma-carboxyglutamic acid
  MOD_RES 8: note = gamma-carboxyglutamic acid
XWWWXWWX

SEQ ID NO: 311 - protein, 8 aa, synthetic construct
  REGION 1..8: note = Description of Artificial Sequence: Synthetic peptide
  MOD_RES 1: note = gamma-carboxyglutamic acid
  MOD_RES 6: note = gamma-carboxyglutamic acid
  MOD_RES 8: note = gamma-carboxyglutamic acid
XWWWWXWX

SEQ ID NO: 312 - protein, 8 aa, synthetic construct
  REGION 1..8: note = Description of Artificial Sequence: Synthetic peptide
  MOD_RES 1: note = gamma-carboxyglutamic acid
  MOD_RES 7..8: note = gamma-carboxyglutamic acid
XWWWWWXX

SEQ ID NO: 313 - protein, 8 aa, synthetic construct
  REGION 1..8: note = Description of Artificial Sequence: Synthetic peptide
  MOD_RES 1: note = gamma-carboxyglutamic acid
  MOD_RES 3..4: note = gamma-carboxyglutamic acid
XWXXWWWW

SEQ ID NO: 314 - protein, 8 aa, synthetic construct
  REGION 1..8: note = Description of Artificial Sequence: Synthetic peptide
  MOD_RES 1: note = gamma-carboxyglutamic acid
  MOD_RES 4..5: note = gamma-carboxyglutamic acid
XWWXXWWW

SEQ ID NO: 315 - protein, 8 aa, synthetic construct
  REGION 1..8: note = Description of Artificial Sequence: Synthetic peptide
  MOD_RES 1: note = gamma-carboxyglutamic acid
  MOD_RES 5..6: note = gamma-carboxyglutamic acid
XWWWXXWW

SEQ ID NO: 316 - protein, 8 aa, synthetic construct
  REGION 1..8: note = Description of Artificial Sequence: Synthetic peptide
  MOD_RES 1: note = gamma-carboxyglutamic acid
  MOD_RES 6..7: note = gamma-carboxyglutamic acid
XWWWWXXW

SEQ ID NO: 317 - protein, 8 aa, synthetic construct
  REGION 1..8: note = Description of Artificial Sequence: Synthetic peptide
  MOD_RES 2..4: note = gamma-carboxyglutamic acid
WXXXWWWW

SEQ ID NO: 318 - protein, 8 aa, synthetic construct
  REGION 1..8: note = Description of Artificial Sequence: Synthetic peptide
  MOD_RES 3..5: note = gamma-carboxyglutamic acid
WWXXXWWW

SEQ ID NO: 319 - protein, 8 aa, synthetic construct
  REGION 1..8: note = Description of Artificial Sequence: Synthetic peptide
  MOD_RES 4..6: note = gamma-carboxyglutamic acid
WWWXXXWW

SEQ ID NO: 320 - protein, 8 aa, synthetic construct
  REGION 1..8: note = Description of Artificial Sequence: Synthetic peptide
  MOD_RES 5..7: note = gamma-carboxyglutamic acid
WWWWXXXW

SEQ ID NO: 321 - protein, 8 aa, synthetic construct
  REGION 1..8: note = Description of Artificial Sequence: Synthetic peptide
  MOD_RES 2: note = gamma-carboxyglutamic acid
  MOD_RES 4..5: note = gamma-carboxyglutamic acid
WXWXXWWW

SEQ ID NO: 322 - protein, 8 aa, synthetic construct
  REGION 1..8: note = Description of Artificial Sequence: Synthetic peptide
  MOD_RES 2: note = gamma-carboxyglutamic acid
  MOD_RES 5..6: note = gamma-carboxyglutamic acid
WXWWXXWW

SEQ ID NO: 323 - protein, 8 aa, synthetic construct
  REGION 1..8: note = Description of Artificial Sequence: Synthetic peptide
  MOD_RES 2: note = gamma-carboxyglutamic acid
  MOD_RES 6..7: note = gamma-carboxyglutamic acid
WXWWWXXW

SEQ ID NO: 324 - protein, 8 aa, synthetic construct
  REGION 1..8: note = Description of Artificial Sequence: Synthetic peptide
  MOD_RES 2: note = gamma-carboxyglutamic acid
  MOD_RES 7..8: note = gamma-carboxyglutamic acid
WXWWWWXX

SEQ ID NO: 325 - protein, 8 aa, synthetic construct
  REGION 1..8: note = Description of Artificial Sequence: Synthetic peptide
EEEWWWWC

SEQ ID NO: 326 - protein, 8 aa, synthetic construct
  REGION 1..8: note = Description of Artificial Sequence: Synthetic peptide
EWEWWWEC

SEQ ID NO: 327 - protein, 8 aa, synthetic construct
  REGION 1..8: note = Description of Artificial Sequence: Synthetic peptide
EWWEWWEC

SEQ ID NO: 328 - protein, 8 aa, synthetic construct
  REGION 1..8: note = Description of Artificial Sequence: Synthetic peptide
EWWWEWEC

SEQ ID NO: 329 - protein, 8 aa, synthetic construct
  REGION 1..8: note = Description of Artificial Sequence: Synthetic peptide
EWWWWEEC

SEQ ID NO: 330 - protein, 8 aa, synthetic construct
  REGION 1..8: note = Description of Artificial Sequence: Synthetic peptide
EWEEWWWC

SEQ ID NO: 331 - protein, 8 aa, synthetic construct
  REGION 1..8: note = Description of Artificial Sequence: Synthetic peptide
EWWEEWWC

SEQ ID NO: 332 - protein, 8 aa, synthetic construct
  REGION 1..8: note = Description of Artificial Sequence: Synthetic peptide
EWWWEEWC

SEQ ID NO: 333 - protein, 8 aa, synthetic construct
  REGION 1..8: note = Description of Artificial Sequence: Synthetic peptide
EWWWWEEC

SEQ ID NO: 334 - protein, 8 aa, synthetic construct
  REGION 1..8: note = Description of Artificial Sequence: Synthetic peptide
WEEEWWWC

SEQ ID NO: 335 - protein, 8 aa, synthetic construct
  REGION 1..8: note = Description of Artificial Sequence: Synthetic peptide
WWEEEWWC

SEQ ID NO: 336 - protein, 8 aa, synthetic construct
  REGION 1..8: note = Description of Artificial Sequence: Synthetic peptide
WWWEEEWC

SEQ ID NO: 337 - protein, 8 aa, synthetic construct
  REGION 1..8: note = Description of Artificial Sequence: Synthetic peptide
WWWWEEEC

SEQ ID NO: 338 - protein, 8 aa, synthetic construct
  REGION 1..8: note = Description of Artificial Sequence: Synthetic peptide
WEWEEWWC

SEQ ID NO: 339 - protein, 8 aa, synthetic construct
  REGION 1..8: note = Description of Artificial Sequence: Synthetic peptide
WEWWEEWC

SEQ ID NO: 340 - protein, 8 aa, synthetic construct
  REGION 1..8: note = Description of Artificial Sequence: Synthetic peptide
WEWWWEEC

SEQ ID NO: 341 - protein, 7 aa, synthetic construct
  REGION 1..7: note = Description of Artificial Sequence: Synthetic peptide
EEEWWWW

SEQ ID NO: 342 - protein, 7 aa, synthetic construct
  REGION 1..7: note = Description of Artificial Sequence: Synthetic peptide
EWEWWWE

SEQ ID NO: 343 - protein, 7 aa, synthetic construct
  REGION 1..7: note = Description of Artificial Sequence: Synthetic peptide
EWWEWWE

SEQ ID NO: 344 - protein, 7 aa, synthetic construct
  REGION 1..7: note = Description of Artificial Sequence: Synthetic peptide
EWWWEWE

SEQ ID NO: 345 - protein, 7 aa, synthetic construct
  REGION 1..7: note = Description of Artificial Sequence: Synthetic peptide
EWWWWEE

SEQ ID NO: 346 - protein, 7 aa, synthetic construct
  REGION 1..7: note = Description of Artificial Sequence: Synthetic peptide
EWEEWWW

SEQ ID NO: 347 - protein, 7 aa, synthetic construct
  REGION 1..7: note = Description of Artificial Sequence: Synthetic peptide
EWWEEWW

SEQ ID NO: 348 - protein, 7 aa, synthetic construct
  REGION 1..7: note = Description of Artificial Sequence: Synthetic peptide
EWWWEEW

SEQ ID NO: 349 - protein, 7 aa, synthetic construct
  REGION 1..7: note = Description of Artificial Sequence: Synthetic peptide
EWWWWEE

SEQ ID NO: 350 - protein, 7 aa, synthetic construct
  REGION 1..7: note = Description of Artificial Sequence: Synthetic peptide
WEEEWWW

SEQ ID NO: 351 - protein, 7 aa, synthetic construct
  REGION 1..7: note = Description of Artificial Sequence: Synthetic peptide
WWEEEWW

SEQ ID NO: 352 - protein, 7 aa, synthetic construct
  REGION 1..7: note = Description of Artificial Sequence: Synthetic peptide
WWWEEEW

SEQ ID NO: 353 - protein, 7 aa, synthetic construct
  REGION 1..7: note = Description of Artificial Sequence: Synthetic peptide
WWWWEEE

SEQ ID NO: 354 - protein, 7 aa, synthetic construct
  REGION 1..7: note = Description of Artificial Sequence: Synthetic peptide
WEWEEWW

SEQ ID NO: 355 - protein, 7 aa, synthetic construct
  REGION 1..7: note = Description of Artificial Sequence: Synthetic peptide
WEWWEEW

SEQ ID NO: 356 - protein, 7 aa, synthetic construct
  REGION 1..7: note = Description of Artificial Sequence: Synthetic peptide
WEWWWEE

SEQ ID NO: 357 - protein, 8 aa, synthetic construct
  REGION 1..8: note = Description of Artificial Sequence: Synthetic peptide
DDDWWWWC

SEQ ID NO: 358 - protein, 8 aa, synthetic construct
  REGION 1..8: note = Description of Artificial Sequence: Synthetic peptide
DWDWWWDC

SEQ ID NO: 359 - protein, 8 aa, synthetic construct
  REGION 1..8: note = Description of Artificial Sequence: Synthetic peptide
DWWDWWDC

SEQ ID NO: 360 - protein, 8 aa, synthetic construct
  REGION 1..8: note = Description of Artificial Sequence: Synthetic peptide
DWWWDWDC

SEQ ID NO: 361 - protein, 8 aa, synthetic construct
  REGION 1..8: note = Description of Artificial Sequence: Synthetic peptide
DWWWWDDC

SEQ ID NO: 362 - protein, 8 aa, synthetic construct
  REGION 1..8: note = Description of Artificial Sequence: Synthetic peptide
DWDDWWWC

SEQ ID NO: 363 - protein, 8 aa, synthetic construct
  REGION 1..8: note = Description of Artificial Sequence: Synthetic peptide
DWWDDWWC

SEQ ID NO: 364 - protein, 8 aa, synthetic construct
  REGION 1..8: note = Description of Artificial Sequence: Synthetic peptide
DWWWDDWC

SEQ ID NO: 365 - protein, 8 aa, synthetic construct
  REGION 1..8: note = Description of Artificial Sequence: Synthetic peptide
DWWWWDDC

SEQ ID NO: 366 - protein, 8 aa, synthetic construct
  REGION 1..8: note = Description of Artificial Sequence: Synthetic peptide
WDDDWWWC

SEQ ID NO: 367 - protein, 8 aa, synthetic construct
  REGION 1..8: note = Description of Artificial Sequence: Synthetic peptide
WWDDDWWC

SEQ ID NO: 368 - protein, 8 aa, synthetic construct
  REGION 1..8: note = Description of Artificial Sequence: Synthetic peptide
WWWDDDWC

SEQ ID NO: 369 - protein, 8 aa, synthetic construct
  REGION 1..8: note = Description of Artificial Sequence: Synthetic peptide
WWWWDDDC

SEQ ID NO: 370 - protein, 8 aa, synthetic construct
  REGION 1..8: note = Description of Artificial Sequence: Synthetic peptide
WDWDDWWC

SEQ ID NO: 371 - protein, 8 aa, synthetic construct
  REGION 1..8: note = Description of Artificial Sequence: Synthetic peptide
WDWWDDWC

SEQ ID NO: 372 - protein, 8 aa, synthetic construct
  REGION 1..8: note = Description of Artificial Sequence: Synthetic peptide
WDWWWDDC

SEQ ID NO: 373 - protein, 7 aa, synthetic construct
  REGION 1..7: note = Description of Artificial Sequence: Synthetic peptide
DDDWWWW

SEQ ID NO: 374 - protein, 7 aa, synthetic construct
  REGION 1..7: note = Description of Artificial Sequence: Synthetic peptide
DWDWWWD

SEQ ID NO: 375 - protein, 7 aa, synthetic construct
  REGION 1..7: note = Description of Artificial Sequence: Synthetic peptide
DWWDWWD

SEQ ID NO: 376 - protein, 7 aa, synthetic construct
  REGION 1..7: note = Description of Artificial Sequence: Synthetic peptide
DWWWDWD

SEQ ID NO: 377 - protein, 7 aa, synthetic construct
  REGION 1..7: note = Description of Artificial Sequence: Synthetic peptide
DWWWWDD

SEQ ID NO: 378 - protein, 7 aa, synthetic construct
  REGION 1..7: note = Description of Artificial Sequence: Synthetic peptide
DWDDWWW

SEQ ID NO: 379 - protein, 7 aa, synthetic construct
  REGION 1..7: note = Description of Artificial Sequence: Synthetic peptide
DWWDDWW

SEQ ID NO: 380 - protein, 7 aa, synthetic construct
  REGION 1..7: note = Description of Artificial Sequence: Synthetic peptide
DWWWDDW

SEQ ID NO: 381 - protein, 7 aa, synthetic construct
  REGION 1..7: note = Description of Artificial Sequence: Synthetic peptide
DWWWWDD

SEQ ID NO: 382 - protein, 7 aa, synthetic construct
  REGION 1..7: note = Description of Artificial Sequence: Synthetic peptide
WDDDWWW

SEQ ID NO: 383 - protein, 7 aa, synthetic construct
  REGION 1..7: note = Description of Artificial Sequence: Synthetic peptide
WWDDDWW

SEQ ID NO: 384 - skipped

SEQ ID NO: 385 - protein, 7 aa, synthetic construct
  REGION 1..7: note = Description of Artificial Sequence: Synthetic peptide
WWWDDDW

SEQ ID NO: 386 - protein, 7 aa, synthetic construct
  REGION 1..7: note = Description of Artificial Sequence: Synthetic peptide
WWWWDDD

SEQ ID NO: 387 - protein, 7 aa, synthetic construct
  REGION 1..7: note = Description of Artificial Sequence: Synthetic peptide
WDWDDWW

SEQ ID NO: 388 - protein, 7 aa, synthetic construct
  REGION 1..7: note = Description of Artificial Sequence: Synthetic peptide
WDWWDDW

SEQ ID NO: 389-398 - skipped (10 entries)

SEQ ID NO: 399 - protein, 7 aa, synthetic construct
  REGION 1..7: note = Description of Artificial Sequence: Synthetic peptide
WDWWWDD

SEQ ID NO: 400 - protein, 7 aa, synthetic construct
  REGION 1..7: note = Description of Artificial Sequence: Synthetic peptide
  MOD_RES 1..3: note = gamma-carboxyglutamic acid
XXXWWWW

SEQ ID NO: 401 - protein, 7 aa, synthetic construct
  REGION 1..7: note = Description of Artificial Sequence: Synthetic peptide
  MOD_RES 1: note = gamma-carboxyglutamic acid
  MOD_RES 3: note = gamma-carboxyglutamic acid
  MOD_RES 7: note = gamma-carboxyglutamic acid
XWXWWWX

SEQ ID NO: 402 - protein, 7 aa, synthetic construct
  REGION 1..7: note = Description of Artificial Sequence: Synthetic peptide
  MOD_RES 1: note = gamma-carboxyglutamic acid
  MOD_RES 4: note = gamma-carboxyglutamic acid
  MOD_RES 7: note = gamma-carboxyglutamic acid
XWWXWWX

SEQ ID NO: 403 - protein, 7 aa, synthetic construct
  REGION 1..7: note = Description of Artificial Sequence: Synthetic peptide
  MOD_RES 1: note = gamma-carboxyglutamic acid
  MOD_RES 5: note = gamma-carboxyglutamic acid
  MOD_RES 7: note = gamma-carboxyglutamic acid
XWWWXWX

SEQ ID NO: 404 - protein, 7 aa, synthetic construct
  REGION 1..7: note = Description of Artificial Sequence: Synthetic peptide
  MOD_RES 1: note = gamma-carboxyglutamic acid
  MOD_RES 6..7: note = gamma-carboxyglutamic acid
XWWWWXX

SEQ ID NO: 405 - protein, 7 aa, synthetic construct
  REGION 1..7: note = Description of Artificial Sequence: Synthetic peptide
  MOD_RES 1: note = gamma-carboxyglutamic acid
  MOD_RES 3..4: note = gamma-carboxyglutamic acid
XWXXWWW

SEQ ID NO: 406 - protein, 7 aa, synthetic construct
  REGION 1..7: note = Description of Artificial Sequence: Synthetic peptide
  MOD_RES 1: note = gamma-carboxyglutamic acid
  MOD_RES 4..5: note = gamma-carboxyglutamic acid
XWWXXWW

SEQ ID NO: 407 - protein, 7 aa, synthetic construct
  REGION 1..7: note = Description of Artificial Sequence: Synthetic peptide
  MOD_RES 1: note = gamma-carboxyglutamic acid
  MOD_RES 5..6: note = gamma-carboxyglutamic acid
XWWWXXW

SEQ ID NO: 408 - protein, 7 aa, synthetic construct
  REGION 1..7: note = Description of Artificial Sequence: Synthetic peptide
  MOD_RES 1: note = gamma-carboxyglutamic acid
  MOD_RES 6..7: note = gamma-carboxyglutamic acid
XWWWWXX

SEQ ID NO: 409 - protein, 7 aa, synthetic construct
  REGION 1..7: note = Description of Artificial Sequence: Synthetic peptide
  MOD_RES 2..4: note = gamma-carboxyglutamic acid
WXXXWWW

SEQ ID NO: 410 - protein, 7 aa, synthetic construct
  REGION 1..7: note = Description of Artificial Sequence: Synthetic peptide
  MOD_RES 3..5: note = gamma-carboxyglutamic acid
WWXXXWW

SEQ ID NO: 411 - protein, 7 aa, synthetic construct
  REGION 1..7: note = Description of Artificial Sequence: Synthetic peptide
  MOD_RES 4..6: note = gamma-carboxyglutamic acid
WWWXXXW

SEQ ID NO: 412 - protein, 7 aa, synthetic construct
  REGION 1..7: note = Description of Artificial Sequence: Synthetic peptide
  MOD_RES 5..7: note = gamma-carboxyglutamic acid
WWWWXXX

SEQ ID NO: 413 - protein, 7 aa, synthetic construct
  REGION 1..7: note = Description of Artificial Sequence: Synthetic peptide
  MOD_RES 2: note = gamma-carboxyglutamic acid
  MOD_RES 4..5: note = gamma-carboxyglutamic acid
WXWXXWW

SEQ ID NO: 414 - protein, 7 aa, synthetic construct
  REGION 1..7: note = Description of Artificial Sequence: Synthetic peptide
  MOD_RES 2: note = gamma-carboxyglutamic acid
  MOD_RES 5..6: note = Gla
WXWWXXW

SEQ ID NO: 415 - protein, 7 aa, synthetic construct
  REGION 1..7: note = Description of Artificial Sequence: Synthetic peptide
  MOD_RES 2: note = gamma-carboxyglutamic acid
  MOD_RES 6..7: note = gamma-carboxyglutamic acid
WXWWWXX

SEQ ID NO: 416 - protein, 8 aa, synthetic construct
  REGION 1..8: note = Description of Artificial Sequence: Synthetic peptide
WEWEWEWC

SEQ ID NO: 417 - protein, 8 aa, synthetic construct
  REGION 1..8: note = Description of Artificial Sequence: Synthetic peptide
EWEWEWWC

SEQ ID NO: 418 - protein, 8 aa, synthetic construct
  REGION 1..8: note = Description of Artificial Sequence: Synthetic peptide
WDWDWDWC

SEQ ID NO: 419 - protein, 8 aa, synthetic construct
  REGION 1..8: note = Description of Artificial Sequence: Synthetic peptide
DWDWDWWC

SEQ ID NO: 420 - protein, 8 aa, synthetic construct
  REGION 1..8: note = Description of Artificial Sequence: Synthetic peptide
  MOD_RES 2: note = gamma-carboxyglutamic acid
  MOD_RES 4: note = gamma-carboxyglutamic acid
WXWXWDWC

SEQ ID NO: 421 - protein, 8 aa, synthetic construct
  REGION 1..8: note = Description of Artificial Sequence: Synthetic peptide
DWDWDWDC

SEQ ID NO: 422 - protein, 8 aa, synthetic construct
  REGION 1..8: note = Description of Artificial Sequence: Synthetic peptide
WEWEWEWE

SEQ ID NO: 423 - protein, 8 aa, synthetic construct
  REGION 1..8: note = Description of Artificial Sequence: Synthetic peptide
EWEWEWEW

SEQ ID NO: 424 - protein, 8 aa, synthetic construct
  REGION 1..8: note = Description of Artificial Sequence: Synthetic peptide
WDWDWDWD

SEQ ID NO: 425 - protein, 8 aa, synthetic construct
  REGION 1..8: note = Description of Artificial Sequence: Synthetic peptide
DWDWDWDW

SEQ ID NO: 426 - protein, 8 aa, synthetic construct
  REGION 1..8: note = Description of Artificial Sequence: Synthetic peptide
  MOD_RES 2: note = gamma-carboxyglutamic acid
  MOD_RES 4: note = gamma-carboxyglutamic acid
  MOD_RES 6: note = gamma-carboxyglutamic acid
  MOD_RES 8: note = gamma-carboxyglutamic acid
WXWXWXWX

SEQ ID NO: 427 - protein, 8 aa, synthetic construct
  REGION 1..8: note = Description of Artificial Sequence: Synthetic peptide
  MOD_RES 1: note = gamma-carboxyglutamic acid
  MOD_RES 3: note = gamma-carboxyglutamic acid
  MOD_RES 5: note = gamma-carboxyglutamic acid
  MOD_RES 7: note = gamma-carboxyglutamic acid
XWXWXWXW

SEQ ID NO: 428 - protein, 10 aa, synthetic construct
  REGION 1..10: note = Description of Artificial Sequence: Synthetic peptide
CWEWEWEWEW

SEQ ID NO: 429 - protein, 8 aa, synthetic construct
  REGION 1..8: note = Description of Artificial Sequence: Synthetic peptide
  MOD_RES 2: note = gamma-carboxyglutamic acid
  MOD_RES 4: note = gamma-carboxyglutamic acid
WXWXWDWC

SEQ ID NO: 430 - protein, 8 aa, synthetic construct
  REGION 1..8: note = Description of Artificial Sequence: Synthetic peptide
EWEWEWEC

SEQ ID NO: 431 - protein, 8 aa, synthetic construct
  REGION 1..8: note = Description of Artificial Sequence: Synthetic peptide
DWDWDWDC

SEQ ID NO: 432 - protein, 6 aa, synthetic construct
  REGION 1..6: note = Description of Artificial Sequence: Synthetic peptide
EEEEEK

SEQ ID NO: 433 - protein, 6 aa, synthetic construct
  REGION 1..6: note = Description of Artificial Sequence: Synthetic peptide
WWWWWC

SEQ ID NO: 434 - protein, 5 aa, synthetic construct
  REGION 1..5: note = Description of Artificial Sequence: Synthetic peptide
EEEEK

SEQ ID NO: 435 - protein, 6 aa, synthetic construct
  REGION 1..6: note = Description of Artificial Sequence: Synthetic peptide
WWWWWC

SEQ ID NO: 436 - protein, 6 aa, synthetic construct
  REGION 1..6: note = Description of Artificial Sequence: Synthetic peptide
EEEEEK

SEQ ID NO: 437 - protein, 5 aa, synthetic construct
  REGION 1..5: note = Description of Artificial Sequence: Synthetic peptide
WWWWC

SEQ ID NO: 438 - protein, 5 aa, synthetic construct
  REGION 1..5: note = Description of Artificial Sequence: Synthetic peptide
EEEEK

SEQ ID NO: 439 - protein, 5 aa, synthetic construct
  REGION 1..5: note = Description of Artificial Sequence: Synthetic peptide
WWWWC

SEQ ID NO: 440 - protein, 6 aa, synthetic construct
  REGION 1..6: note = Description of Artificial Sequence: Synthetic peptide
EEEEEK

SEQ ID NO: 441 - protein, 5 aa, synthetic construct
  REGION 1..5: note = Description of Artificial Sequence: Synthetic peptide
WWWWW

SEQ ID NO: 442 - protein, 5 aa, synthetic construct
  REGION 1..5: note = Description of Artificial Sequence: Synthetic peptide
EEEEK

SEQ ID NO: 443 - protein, 5 aa, synthetic construct
  REGION 1..5: note = Description of Artificial Sequence: Synthetic peptide
WWWWW

SEQ ID NO: 444 - protein, 6 aa, synthetic construct
  REGION 1..6: note = Description of Artificial Sequence: Synthetic peptide
EEEEEK

SEQ ID NO: 445 - protein, 4 aa, synthetic construct
  REGION 1..4: note = Description of Artificial Sequence: Synthetic peptide
WWWW

SEQ ID NO: 446 - protein, 5 aa, synthetic construct
  REGION 1..5: note = Description of Artificial Sequence: Synthetic peptide
EEEEK

SEQ ID NO: 447 - protein, 4 aa, synthetic construct
  REGION 1..4: note = Description of Artificial Sequence: Synthetic peptide
WWWW

SEQ ID NO: 448 - protein, 6 aa, synthetic construct
  REGION 1..6: note = Description of Artificial Sequence: Synthetic peptide
DDDDDK

SEQ ID NO: 449 - protein, 6 aa, synthetic construct
  REGION 1..6: note = Description of Artificial Sequence: Synthetic peptide
WWWWWC

SEQ ID NO: 450 - protein, 5 aa, synthetic construct
  REGION 1..5: note = Description of Artificial Sequence: Synthetic peptide
DDDDK

SEQ ID NO: 451 - protein, 6 aa, synthetic construct
  REGION 1..6: note = Description of Artificial Sequence: Synthetic peptide
WWWWWC

SEQ ID NO: 452 - protein, 6 aa, synthetic construct
  REGION 1..6: note = Description of Artificial Sequence: Synthetic peptide
DDDDDK

SEQ ID NO: 453 - protein, 5 aa, synthetic construct
  REGION 1..5: note = Description of Artificial Sequence: Synthetic peptide
WWWWC

SEQ ID NO: 454 - protein, 5 aa, synthetic construct
  REGION 1..5: note = Description of Artificial Sequence: Synthetic peptide
DDDDK

SEQ ID NO: 455 - protein, 5 aa, synthetic construct
  REGION 1..5: note = Description of Artificial Sequence: Synthetic peptide
WWWWC

SEQ ID NO: 456 - protein, 6 aa, synthetic construct
  REGION 1..6: note = Description of Artificial Sequence: Synthetic peptide
DDDDDK

SEQ ID NO: 457 - protein, 5 aa, synthetic construct
  REGION 1..5: note = Description of Artificial Sequence: Synthetic peptide
WWWWW

SEQ ID NO: 458 - protein, 5 aa, synthetic construct
  REGION 1..5: note = Description of Artificial Sequence: Synthetic peptide
DDDDK

SEQ ID NO: 459 - protein, 5 aa, synthetic construct
  REGION 1..5: note = Description of Artificial Sequence: Synthetic peptide
WWWWW

SEQ ID NO: 460 - protein, 6 aa, synthetic construct
  REGION 1..6: note = Description of Artificial Sequence: Synthetic peptide
DDDDDK

SEQ ID NO: 461 - protein, 4 aa, synthetic construct
  REGION 1..4: note = Description of Artificial Sequence: Synthetic peptide
WWWW

SEQ ID NO: 462 - protein, 5 aa, synthetic construct
  REGION 1..5: note = Description of Artificial Sequence: Synthetic peptide
DDDDK

SEQ ID NO: 463 - protein, 4 aa, synthetic construct
  REGION 1..4: note = Description of Artificial Sequence: Synthetic peptide
WWWW

SEQ ID NO: 464 - skipped

SEQ ID NO: 465 - protein, 6 aa, synthetic construct
  REGION 1..6: note = Description of Artificial Sequence: Synthetic peptide
WWWWWC

SEQ ID NO: 466 - skipped

SEQ ID NO: 467 - protein, 6 aa, synthetic construct
  REGION 1..6: note = Description of Artificial Sequence: Synthetic peptide
WWWWWC

SEQ ID NO: 468 - skipped

SEQ ID NO: 469 - protein, 5 aa, synthetic construct
  REGION 1..5: note = Description of Artificial Sequence: Synthetic peptide
WWWWC

SEQ ID NO: 470 - skipped

SEQ ID NO: 471 - protein, 5 aa, synthetic construct
  REGION 1..5: note = Description of Artificial Sequence: Synthetic peptide
WWWWC

SEQ ID NO: 472 - skipped

SEQ ID NO: 473 - protein, 5 aa, synthetic construct
  REGION 1..5: note = Description of Artificial Sequence: Synthetic peptide
WWWWW

SEQ ID NO: 474 - skipped

SEQ ID NO: 475 - protein, 5 aa, synthetic construct
  REGION 1..5: note = Description of Artificial Sequence: Synthetic peptide
WWWWW

SEQ ID NO: 476 - skipped

SEQ ID NO: 477 - protein, 4 aa, synthetic construct
  REGION 1..4: note = Description of Artificial Sequence: Synthetic peptide
WWWW

SEQ ID NO: 478 - skipped

SEQ ID NO: 479 - protein, 4 aa, synthetic construct
  REGION 1..4: note = Description of Artificial Sequence: Synthetic peptide
WWWW

SEQ ID NO: 480 - protein, 11 aa, synthetic construct
  REGION 1..11: note = Description of Artificial Sequence: Synthetic peptide
EEEEEWWWWWC

SEQ ID NO: 481 - protein, 9 aa, synthetic construct
  REGION 1..9: note = Description of Artificial Sequence: Synthetic peptide
EEEEWWWWC

SEQ ID NO: 482 - protein, 10 aa, synthetic construct
  REGION 1..10: note = Description of Artificial Sequence: Synthetic peptide
WEWEWEWECW

SEQ ID NO: 483 - protein, 10 aa, synthetic construct
  REGION 1..10: note = Description of Artificial Sequence: Synthetic peptide
WRWRWRWRWC

SEQ ID NO: 484 - protein, 26 aa, synthetic construct
  REGION 1..26: note = Description of Artificial Sequence: Synthetic peptide
  MOD_RES 12: note = Aad
  MOD_RES 23: note = Aad
ADDQNPWRAYLXLLFPTDTLLLXLLW

SEQ ID NO: 485 - protein, 1225 aa, Homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 486 - DNA, 4889 bp, Homo sapiens
  source 1..4889: mol_type = other DNA
aagttcctgtgttctttattctactctccgctgaagtccacacagtttaaattaaagttcccggatttttgtgggcgcctgccccgcccctcgtccccctgctgtgtccatatatcgaggcgatagggttaagggaaggcggacgcctgatgggttaatgagcaaactgaagtgttttccatgatcttttttgagtcgcaattgaagtaccacctcccgagggtgattgcttccccatgcggggtagaacctttgctgtcctgttcaccactctacctccagcacagaatttggcttatgcctactcaatgtgaagatgatgaggatgaaaacctttgtgatgatccacttccacttaatgaatggtggcaaagcaaagctatattcaagaccacatgcaaagctactccctgagcaaagagtcacagataaaacgggggcaccagtagaatggccaggacaaacgcagtgcagcacagagactcagaccctggcagccatgcctgcgcaggcagtgatgagagtgacatgtactgttgtggacatgcacaaaagtgagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaaaaaaaaaaaaaaaaaa

SEQ ID NO: 487 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 488 - DNA, 1029 bp, Homo sapiens
  source 1..1029: mol_type = other DNA
ctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaa

SEQ ID NO: 489 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 490 - DNA, 1127 bp, Homo sapiens
  source 1..1127: mol_type = other DNA
attctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattca

SEQ ID NO: 491 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 492 - DNA, 2016 bp, Homo sapiens
  source 1..2016: mol_type = other DNA
acacttgtggcttccgtgcacacattaacaactcatggttctagctcccagtcgccaagcgttgccaaggcgttgagagatcatctgggaagtcttttacccagaattgctttgattcaggccagctggtttttcctgcggtgattcggaaattcgcgaattcctctggtcctcatccaggtgcgcgggaagcaggtgcccaggagagaggggataatgaagattccatgctgatgatcccaaagattgaacctgcagaccaagcgcaaagtagaaactgaaagtacactgctggcggatcctacggaagttatggaaaaggcaaagcgcagagccacgccgtagtgtgtgccgccccccttgggatggatgaaactgcagtcgcggcgtgggtaagaggaaccagctgcagagatcaccctgcccaacacagactcggcaactccgcggaagaccagggtcctgggagtgactatgggcggtgagagcttgctcctgctccagttgcggtcatcatgactacgcccgcctcccgcagaccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactgaaaaatatggagtggcaatatagaaacacgaactttagctgcatcctccaagaatctatctgcttatgcagtttttcagagtggaatgcttcctagaagttactgaatgcaccatggtcaaaacggattagggcatttgagaaatgcatattgtattactagaagatgaatacaaacaatggaaactgaatgctccagtcaacaaactatttcttatatatgtgaacatttatcaatcagtataattctgtactgatttttgtaagacaatccatgtaaggtatcagttgcaataatacttctcaaacctgtttaaatatttcaagacattaaatctatgaagtatataatggtttcaaagattcaaaattgacattgctttactgtcaaaataattttatggctcactatgaatctattatactgtattaagagtgaaaattgtcttcttctgtgctggagatgttttagagttaacaatgatatatggataatgccggtgagaataagagagtcataaaccttaagtaagcaacagcataacaaggtccaagatacctaaaagagatttcaagagatttaattaatcatgaatgtgtaacacagtgccttcaataaatggtatagcaaatgttttgacatgaaaaaaggacaatttcaaaaaaataaaataaaataaaaataaattcacctagtctaaggatgctaaaccttagtactgagttacattgtcatttatatagattataacttgtctaaataagtttgcaatttgggagatatatttttaagataataatatatgtttaccttttaattaatgaaatatctgtatttaattttgacactatatctgtatataaaatattttcatacagcattacaaattgcttactttggaatacatttctcctttgataaaataaatgagctatgtattaa

SEQ ID NO: 493 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 494 - DNA, 762 bp, Homo sapiens
  source 1..762: mol_type = other DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 495 - protein, 28 aa, synthetic construct
  REGION 1..28: note = Description of Artificial Sequence: Synthetic peptide
  REGION 1..28: note = See specification as filed for detailed description of substitutions and preferred embodiments
  SITE 2: note = Lys, Cys or an Azido-containing amino acid
AXDQDNPWRAYLDLLFPTDTLLLDLLWA

SEQ ID NO: 496 - protein, 35 aa, synthetic construct
  REGION 1..35: note = Description of Artificial Sequence: Synthetic polypeptide
  REGION 1..35: note = See specification as filed for detailed description of substitutions and preferred embodiments
  SITE 2: note = Lys, Cys or an Azido-containing amino acid
  SITE 13: note = Lys, Cys or an Azido-containing amino acid
  REGION 3..12: note = This region may encompass 1-10 residues
AXXXXXXXXXXXXPWRAYLDLLFPTDTLLLDLLWA

SEQ ID NO: 497 - protein, 6 aa, synthetic construct
  REGION 1..6: note = Description of Artificial Sequence: Synthetic peptide
QDNDQN

SEQ ID NO: 498 - DNA, 308 bp, Homo sapiens
  source 1..308: mol_type = other DNA
ccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctcct

SEQ ID NO: 499 - skipped

SEQ ID NO: 500 - DNA, 729 bp, synthetic construct
  misc_feature 1..729: note = Description of Artificial Sequence: Synthetic polynucleotide
  source 1..729: mol_type = other DNA
aatggcggcaccttcgagaagcagatcggcgaggtgaagcccaggaccacccctgccgccgggggaatggacgagtctgtggtcctggagcccgaagccacaggcgaaagcagtagcctggagccgactccttcttcccaggaagcacagagggccctggggacctccccagagctgccgacgggtgtgactggttcctcagggaccaggctccccccgacgccaaaggctcaggatggagggcctgtgggcacggagcttttccgagtgcctcccgtctccactgccgccacgtggcagagttctgctccccaccaacctgggcccagcctctgggctgaggcaaagacctctgaggccccgtccacccaggacccctccacccaggcctccactgcgtcctccccagccccagaggagaatgctccgtctgaaggccagcgtgtgtggggtcaggggcagagccccaggccagagaactctctggagcgggaggagatgggtcccgtgccagcgcacacggatgccttccaggactgggggcctggcagcatggcccacgtctctgtggtccctgtctcctcagaagggacccccagcagggagccagtggcttcaggcagctggacccctaaggctgaggaacccatccatgccaccatggacccccagaggctgggcgtccttatcactcctgtccctgacgcccaggctgccacccggaggcag

SEQ ID NO: 501 - protein, 7 aa, synthetic construct
  REGION 1..7: note = Description of Artificial Sequence: Synthetic peptide
AEQNPIY

SEQ ID NO: 502 - protein, 6 aa, synthetic construct
  REGION 1..6: note = Description of Artificial Sequence: Synthetic peptide
DADEGT

SEQ ID NO: 503 - protein, 28 aa, synthetic construct
  REGION 1..28: note = Description of Artificial Sequence: Synthetic peptide
ACDQDNPWRAYLDLLFPTDTLLLDLLWA

SEQ ID NO: 504 - protein, 28 aa, synthetic construct
  REGION 1..28: note = Description of Artificial Sequence: Synthetic peptide
AKDQDNPWRAYLDLLFPTDTLLLDLLWA

SEQ ID NO: 505 - protein, 31 aa, synthetic construct
  REGION 1..31: note = Description of Artificial Sequence: Synthetic polypeptide
ACQDNDQNCPWRAYLDLLFPTDTLLLDLLWA

SEQ ID NO: 506 - protein, 31 aa, synthetic construct
  REGION 1..31: note = Description of Artificial Sequence: Synthetic polypeptide
AKQDNDQNKPWRAYLDLLFPTDTLLLDLLWA

SEQ ID NO: 507 - protein, 6 aa, synthetic construct
  REGION 1..6: note = Description of Artificial Sequence: Synthetic peptide
WWWWWC

SEQ ID NO: 508 - protein, 5 aa, synthetic construct
  REGION 1..5: note = Description of Artificial Sequence: Synthetic peptide
WWWWC

SEQ ID NO: 509 - protein, 28 aa, synthetic construct
  REGION 1..28: note = Description of Artificial Sequence: Synthetic peptide
ACDDQNPWRAYLDLLFPTDTLLLDLLWA

SEQ ID NO: 510 - protein, 28 aa, synthetic construct
  REGION 1..28: note = Description of Artificial Sequence: Synthetic peptide
AKDDQNPWRAYLDLLFPTDTLLLDLLWA

SEQ ID NO: 511 - protein, 31 aa, synthetic construct
  REGION 1..31: note = Description of Artificial Sequence: Synthetic polypeptide
AKQNDDQNKPWRAYLDLLFPTDTLLLDLLWA

SEQ ID NO: 512 - protein, 14 aa, synthetic construct
  REGION 1..14: note = Description of Artificial Sequence: Synthetic peptide
YPYDVPDYAGGGCA

SEQ ID NO: 513 - protein, 6 aa, synthetic construct
  REGION 1..6: note = Description of Artificial Sequence: Synthetic peptide
QNDDQN

SEQ ID NO: 514 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 515 - protein, 35 aa, synthetic construct; identical to SEQ ID NO: 496
  REGION 1..35: note = Description of Artificial Sequence: Synthetic polypeptide
  REGION 3..12: note = This region may encompass 1-10 residues
  REGION 1..35: note = See specification as filed for detailed description of substitutions and preferred embodiments

SEQ ID NO: 516 - protein, 30 aa, Homo sapiens
MELAALCRWGLLLALLPPGAASTQVCTGTD

SEQ ID NO: 517 - DNA, 1175 bp, Homo sapiens
  source 1..1175: mol_type = other DNA
gagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaa

SEQ ID NO: 518 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 519 - DNA, 2761 bp, Homo sapiens
  source 1..2761: mol_type = other DNA
aaagaatttctcaggctcaaaatccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcagaaaacagatcttgattttttttctcttgaatgtacccttcaactgttgaatgtttaatagtaaatcttatatgtccttatttactttttagctttctctcaaataaagtgtaacactagttgagataacacatgaaagctctttaaagggtcgatcgggaacaggaaaaaaaacctatggaaaatatgacaacac

SEQ ID NO: 520 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 521 - DNA, 1479 bp, Homo sapiens
  source 1..1479: mol_type = other DNA
gttcagcatttctactccttccaagaagagcagcaaagctgaagtagcagcagcagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagagcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgcattatacattcatgcatttctaggctagagaaccttctagatttgatgcttacaactattctgttgtgactatgagaacatttctgtctctagaagtcatctgtctgtattgatctttatgctatattactatctgtggttacggtggagacattgacattattactggagtcaagcccttataagtcaaaagcatctatgtgtcgtaaaacattcctcaaacattttttcatgcaaatacacacttctttccccaaacatcatgtagcacatcaatatgtagggagacattcttatgcatcatttggtttgttttataaccaattcattaaatgtaattcataaaatgtactatgaaaaaaattatacgctatgggatactggcaaaagtgcacatatttcataaccaaattagtagcaccagtcttaatttgatgtttttcaacttttattcattgagatgttttgaagcaattaggatatgtgtgtttactgtactttttgttttgatccgtttgtataaatgatagcaatatcttggacacatctgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaa

SEQ ID NO: 522 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 523 - DNA, 1678 bp, Homo sapiens
  source 1..1678: mol_type = other DNA
agcagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaa

SEQ ID NO: 524 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 525 - DNA, 2364 bp, Homo sapiens
  source 1..2364: mol_type = other DNA
agaagaaacaacatctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccctgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatcata

SEQ ID NO: 526 - protein, 241 aa, Homo sapiens
GTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQ

SEQ ID NO: 527 - protein, 397 aa, Homo sapiens
MAPISLSWLLRLATFCHLTVLLAGQHHGVTKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 528 - protein, 6 aa, synthetic construct
  REGION 1..6: note = Description of Artificial Sequence: Synthetic peptide
NENENN

SEQ ID NO: 529 - protein, 6 aa, synthetic construct
  REGION 1..6: note = Description of Artificial Sequence: Synthetic peptide
NDNDNN

SEQ ID NO: 530 - protein, 7 aa, synthetic construct
  REGION 1..7: note = Description of Artificial Sequence: Synthetic peptide
NDNDNDN

SEQ ID NO: 531 - protein, 385 aa, synthetic construct
  REGION 1..385: note = Description of Artificial Sequence: Synthetic polypeptide
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSPGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQEQKLISEEDLEQKLISEEDLADDQNPWRAYLDLLFPTDTLLLDLLW

SEQ ID NO: 532 - protein, 371 aa, synthetic construct
  REGION 1..371: note = Description of Artificial Sequence: Synthetic polypeptide
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSPGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQHHHHHHADDQNPWRAYLDLLFPTDTLLLDLLW

SEQ ID NO: 533 - protein, 27 aa, synthetic construct
  REGION 1..27: note = Pept
  UNSURE 2: note = X is a functional group, selected from a lysine, a cysteine, or an Azido-containing amino acid
AXDDQNPWRAYLDLLFPTDTLLLDLLW

SEQ ID NO: 534 - protein, 27 aa, synthetic construct
  source 1..27: note = Pept
  UNSURE 2: note = X is a functional group, selected from a lysine, a cysteine, or an Azido-containing amino acid
AXDQDNPWRAYLDLLFPTDTLLLDLLW

SEQ ID NO: 535 - protein, 31 aa, synthetic construct
  source 1..31: note = Pept; note = Pept; note = Pept
AKQNDNDNKPWRAYLDLLFPTDTLLLDLLWA

SEQ ID NO: 536 - protein, 31 aa, synthetic construct
  source 1..31: note = Pept
ACQNDDQNCPWRAYLDLLFPTDTLLLDLLWA

SEQ ID NO: 537 - protein, 28 aa, synthetic construct
  source 1..28: note = Var3-1a
ACDQDNPWRAYLDLLFPTDTLLLDLLWA

SEQ ID NO: 538 - protein, 28 aa, synthetic construct
  REGION 1..28: note = Var3-1b
AKDQDNPWRAYLDLLFPTDTLLLDLLWA

SEQ ID NO: 539 - protein, 31 aa, synthetic construct; identical to SEQ ID NO: 505
  REGION 1..31: note = Var3-2a

SEQ ID NO: 540 - protein, 31 aa, synthetic construct; identical to SEQ ID NO: 506
  source 1..31: note = Var3-2b
  REGION 1..31

SEQ ID NO: 541 - protein, 6 aa, synthetic construct
  source 1..6: note = PEG Conjugation Spacer
QNDDQN

(19) United States
(12) Patent Application Publication
(10) Pub. No.: US 2024/0066096 A1
(43) Pub. Date: Feb. 29, 2024

(54) pHLIP® peptide mediated epitope tethering at cell surfaces

(71) Applicants: UNIVERSITY OF RHODE ISLAND BOARD OF TRUSTEES, Kingston, RI (US); YALE UNIVERSITY, New Haven, CT (US)

(72) Inventors: Yana K. RESHETNYAK, Saunderstown, RI (US); Oleg A. ANDREEV, Saunderstown, RI (US); Anna MOSHNIKOVA, Warwick, RI (US); Donald M. Engelman, New Haven, CT (US)

(21) Appl. No.: 18/325,884

(22) Filed: May 30, 2023

Related U.S. Application Data
(63) Continuation of application No. 16/775,113, filed on Jan. 28, 2020, now abandoned.
(60) Provisional application No. 62/797,899, filed on Jan. 28, 2019.

(51) Int. Cl. A61K 38/16; A61K 38/19; A61K 38/20

(52) U.S. Cl. CPC A61K 38/16; A61K 38/195; A61K 38/20; A61K 47/00; A61K 2121/00; C07K 2319/33

(57) ABSTRACT

The invention features methods and compositions for eliciting an anti-tumor response in a subject comprising administering to the subject a pHLIP^{●} construct comprising an antibody recruiting molecule linked to one or more pHLIP^{●} peptides by a non-cleavable linker compound. The construct increases the amount of the antibody recruiting molecule on the surface of a diseased cell.

### RELATED APPLICATIONS

This application claims the benefit of priority under 35 U.S.C. § 119(e) to U.S. Provisional Application No. 62/797,899, filed Jan. 28, 2019, the entire contents of which is incorporated herein by reference in its entirety.

### STATEMENT AS TO FEDERALLY SPONSORED RESEARCH

This invention was made with government support under R01 GM073857 awarded by the National Institute of General Medical Sciences of the National Institutes of Health. The government has certain rights in the invention.

### INCORPORATION BY REFERENCE OF SEQUENCE LISTING

The contents of the sequence listing text file named “040984-512001WO_SL.txt”, which was created on Feb. 22, 2021 and is 228 kilobytes in size, is hereby incorporated by reference in its entirety.

### FIELD OF THE INVENTION

The present invention relates to immunotherapy.

### BACKGROUND

Antibody therapies are based on the alteration of signaling, promotion of apoptosis, sequestration of growth factors, activation of the immune system, and delivery of drugs as antibody-drug conjugates (ADCs). Antibodies find their target cells by recognizing specific signaling molecules (epitopes) exposed by a cell. Epitopes bind and/or attract endogenous (natural) antibodies, exogenous antibodies, ADCs administrated into the body, or antibodies which are produced (generated) in the course of vaccination. Also cytokines, which are small immune-modulating proteins can activate or suppress an immune response depending on their features, concentration and local environment. Numerous highly specific humanized monoclonal antibodies such as trastuzumab (Herceptin), and antibody-cytokine fusion proteins have been developed and are in clinical use (or clinical trials), which target epitopes that are more abundant in a diseased tissue than in a normal tissue. However, their use is limited by (1) the lack of adequate amounts (or any amounts) of accessible epitopes in many cancers, and (2) the emergence of resistance by selection of expression mutants not presenting proper epitopes.

### SUMMARY OF THE INVENTION

The invention provides a solution to the limitations of existing therapeutic approaches by utilizing a strategy in which a desired epitope is positioned on cell surfaces in diseased tissues, such as tumors or inflamed tissues. Positioning the epitope on the cell surface, (e.g., preferentially in diseased tissues), is a great advantage and enhances the recruitment of cells of the immune system or endogenous antibodies, and enhances the use of developed monoclonal antibodies such as trastuzumab (Herceptin), antibody-drug conjugates, and antibodies generated in the course of vaccination. The invention further provides compositions and methods to augment any amount of particular epitopes at cell surfaces for immunoregulation and efficient binding of immune cells, antibodies and ADCs.

Provided herein are compositions and methods for the decoration of target cells with epitopes, e.g., a protein, a peptide, or a small molecule epitope, that can (1) recruit immune cells (or exogenous engineered T-cells and NK-cells), (2) recruit endogenous antibodies, (3) enhance the use of the exogenous antibodies or ADCs administrated into body, and (4) enhance the use of antibodies, which are produced (generated) in the course of vaccination leading to cell death.

Tumors are characterized by a tumor micro environment (TME) of a lower pH than the surrounding tissues, because of the metabolism accompanying their rapid and uncontrolled cell proliferation, which results in a flux of acidity emerging from the cancer cells. Moreover, due to the flux and the membrane potential, the extracellular pH is lowest at the surfaces of cancer cells and is significantly lower than the bulk extracellular pH in tumors. The low pH region persists at the cancer cell surfaces even in well-perfused tumor areas.

A pH Low Insertion Peptide (pHLIP®) is a water-soluble membrane peptide that interacts weakly with a cell membrane at neutral pH, without insertion into the lipid bilayer; however, at slightly acidic pH (<7.0), a pHLIP® inserts into the cell membrane and, if it is long enough and non-cyclic, can form a stable transmembrane alpha-helix. In addition to tumor cells characterized by low pH (<7.0), immune cells within a tumor mass are also characterized by low pH (<7.0). For example, the cells within the environment of a tumor mass, e.g., macrophages, are also characterized by a low surface pH.

By binding (linking and/or conjugating) a pHLIP®, or pHLIP® equivalent, to an epitope, it is possible to specifically target the cell and decorate a tumor cell or cells in inflamed tissues with epitopes to recognize or recruit endogenous (natural) immune cells or antibodies circulating in the blood, promote and enhance binding of exogenous engineered T-cells and NK-cells or antibodies or ADCs administrated into body, or antibodies, which are generated in the course of vaccination and thereby promote cell killing. A significant advantage of this approach is that the pHLIP® constructs described herein are associated with few to no side effects for the patient due to the targeted delivery of epitopes to the cell surfaces. The epitope may be of mammalian origin, viral origin, or bacterial origin.

Accordingly, the invention features a composition comprising an epitope conjugated to a pH-triggered membrane peptide (pHLIP®) comprising at least 4 amino acids. For example, the pHLIP® peptide may be a linear peptide or a cyclic peptide, e.g., as described in PCT Application No. PCT/US2017/023458. The epitope is selectively positioned on the surface of the cell in targeted diseased tissue by pHLIP® to mediate or enhance immune cells (lymphocytes) recruitment and adhesion, antibody binding and induce cell killing predominantly in diseased tissue (tumor). The composition comprises an epitope conjugated to pHLIP®, and the pHLIP® targets epitope to the cell surface.

Accordingly, the method of eliciting an anti-tumor response in a subject comprises administering to a subject, e.g., a human subject, a pHLIP® construct comprising an antibody or lymphocyte recruiting molecule linked to one or more pHLIP® peptides by a non-cleavable linker compound, wherein the construct increases the amount of the immune cell or the antibody recruiting molecule on the surface of a diseased cell. For example, the diseased cell comprises a tumor cell.

In some examples, the antibody recruiting molecule comprises an epitope, e.g., an epitope (e.g., a peptide epitope) with a length less than 500 amino acids. For example, the peptide epitope comprises a length of between 5 to 20 amino acids, e.g., 5, 6, 7, 8, 9, 10, 11, 12, 13, 14, 15, 16, 17, 18, 19, or 20 amino acids. In some embodiments, the peptide epitope is a human epidermal growth factor 2 (HER2) peptide, e.g., a HER2 peptide selected from the group consisting of

(SEQ ID NO: 1)
QVSHWVSGLAEGSFG,
(SEQ ID NO: 2)
LSHTSGRVEGSVSLL,
and
(SEQ ID NO: 3)
QMWAPQWGPD.

The human amino acid sequence of HER2 is provided below and is incorporated herein by reference (NCBI GenBank NP_001005862.1 or UniProt P04626).

| (SEQ ID NO: 485) | |
|---|---|
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctlvcplhnq evtaedgtqr |
| 301 | cekcskpcar vcyglgmehl revravtsan iqefagckki fgslaflpes fdgdpasnta |
| 361 | plqpeqlqvf etleeitgyl yisawpdslp dlsvfqnlqv irgrilhnga ysltlqglgi |
| 421 | swlglrslre lgsglalihh nthlcfvhtv pwdqlfrnph qallhtanrp edecvgegla |
| 481 | chqlcarghc wgpgptqcvn csqflrgqec veecrvlqgl preyvnarhc lpchpecqpq |
| 541 | ngsvtcfgpe adqcvacahy kdppfcvarc psgvkpdlsy mpiwkfpdee gacqpcpinc |
| 601 | thscvdlddk gcpaeqrasp ltsiisavvg illvvvlgvv fgilikrrqq kirkytmrrl |
| 661 | lqetelvepl tpsgampnqa qmrilketel rkvkvlgsga fgtvykgiwi pdgenvkipv |
| 721 | aikvlrents pkankeilde ayvmagvgsp yvsrllgicl tstvqlvtql mpygclldhv |
| 781 | renrgrlgsq dllnwcmqia kgmsyledvr lvhrdlaarn vlvkspnhvk itdfglarll |
| 841 | dideteyhad ggkvpikwma lesilrrrft hqsdvwsygv tvwelmtfga kpydgipare |
| 901 | ipdllekger lpqppictid vymimvkcwm idsecrprfr elvsefsrma rdpqrfvviq |
| 961 | nedlgpaspl dstfyrslle dddmgdlvda eeylvpqqgf fcpdpapgag gmvhhrhrss |
| 1021 | strsgggdlt lglepseeea prsplapseg agsdvfdgdl gmgaakglqs lpthdpsplq |
| 1081 | rysedptvpl psetdgyvap ltcspqpeyv nqpdvrpqpp spregplpaa rpagatlerp |
| 1141 | ktlspgkngv vkdvfafgga venpeyltpq ggaapqphpp pafspafdnl yywdqdpper |
| 1201 | gappstfkgt ptaenpeylg ldvpv |

Exemplary landmark residues, domains, and fragments of HER2 include, but are not limited to residues 22-143 (Receptor L domain), residues 159-308 (furin like domain), or 611-654 (transmembrane domain of Erb2 (receptor tyrosine-protein kinase). A fragment (e.g., a peptide or an epitope) of a HER2 protein is less than the length of the full length protein, e.g., a fragment is at least 3, 4, 5, 6, 7, 8, 9, 10, 20, 30, 40, 50, 100, 200 or more residues in length, but less than e.g., 1225 residues in the case of HER2 above. Additionally, HER2 may include a signal sequence (melaalcrwglllallppgaastqvctgtd SEQ ID NO: 516, which gets cleaved to form the mature protein).

The human HER2 nucleic acid sequence is provided below and is incorporated herein by reference (start and stop codons are underlined; NCBI GenBank: NM_001005862.2).

| (SEQ ID NO: 486) | |
|---|---|
| 1 | aagttcctgt gttctttatt ctactctccg ctgaagtcca cacagtttaa attaaagttc |
| 61 | ccggattttt gtgggcgcct gccccgcccc tcgtccccct gctgtgtcca tatatcgagg |
| 121 | cgatagggtt aagggaaggc ggacgcctga tgggttaatg agcaaactga agtgttttcc |
| 181 | atgatctttt ttgagtcgca attgaagtac cacctcccga gggtgattgc ttccccatgc |
| 241 | ggggtagaac ctttgctgtc ctgttcacca ctctacctcc agcacagaat ttggcttatg |
| 301 | cctactcaat gtgaagatga tgaggatgaa aacctttgtg atgatccact tccacttaat |
| 361 | gaatggtggc aaagcaaagc tatattcaag accacatgca aagctactcc ctgagcaaag |
| 421 | agtcacagat aaaacggggg caccagtaga atggccagga caaacgcagt gcagcacaga |
| 481 | gactcagacc ctggcagcca tgcctgcgca ggcagtgatg agagtgacat gtactgttgt |
| 541 | ggacatgcac aaaagtgagt gtgcaccggc acagacatga agctgcggct ccctgccagt |
| 601 | cccgagaccc acctggacat gctccgccac ctctaccagg gctgccaggt ggtgcaggga |
| 661 | aacctggaac tcacctacct gcccaccaat gccagcctgt ccttcctgca ggatatccag |
| 721 | gaggtgcagg gctacgtgct catcgctcac aaccaagtga ggcaggtccc actgcagagg |
| 781 | ctgcggattg tgcgaggcac ccagctcttt gaggacaact atgccctggc cgtgctagac |
| 841 | aatggagacc cgctgaacaa taccacccct gtcacagggg cctccccagg aggcctgcgg |
| 901 | gagctgcagc ttcgaagcct cacagagatc ttgaaaggag gggtcttgat ccagcggaac |
| 961 | ccccagctct gctaccagga cacgattttg tggaaggaca tcttccacaa gaacaaccag |
| 1021 | ctggctctca cactgataga caccaaccgc tctcgggcct gccacccctg ttctccgatg |
| 1081 | tgtaagggct cccgctgctg gggagagagt tctgaggatt gtcagagcct gacgcgcact |
| 1141 | gtctgtgccg gtggctgtgc ccgctgcaag gggccactgc ccactgactg ctgccatgag |
| 1201 | cagtgtgctg ccggctgcac gggccccaag cactctgact gcctggcctg cctccacttc |
| 1261 | aaccacagtg gcatctgtga gctgcactgc ccagccctgg tcacctacaa cacagacacg |
| 1321 | tttgagtcca tgcccaatcc cgagggccgg tatacattcg gcgccagctg tgtgactgcc |
| 1381 | tgtccctaca actacctttc tacggacgtg ggatcctgca ccctcgtctg ccccctgcac |
| 1441 | aaccaagagg tgacagcaga ggatggaaca cagcggtgtg agaagtgcag caagccctgt |
| 1501 | gcccgagtgt gctatggtct gggcatggag cacttgcgag aggtgagggc agttaccagt |
| 1561 | gccaatatcc aggagtttgc tggctgcaag aagatctttg ggagcctggc atttctgccg |
| 1621 | gagagctttg atggggaccc agcctccaac actgccccgc tccagccaga gcagctccaa |
| 1681 | gtgtttgaga ctctggaaga gatcacaggt tacctataca tctcagcatg gccggacagc |
| 1741 | ctgcctgacc tcagcgtctt ccagaacctg caagtaatcc ggggacgaat tctgcacaat |
| 1801 | ggcgcctact cgctgaccct gcaagggctg ggcatcagct ggctggggct gcgctcactg |
| 1861 | agggaactgg gcagtggact ggccctcatc caccataaca cccacctctg cttcgtgcac |
| 1921 | acggtgccct gggaccagct ctttcggaac ccgcaccaag ctctgctcca cactgccaac |
| 1981 | cggccagagg acgagtgtgt gggcgagggc ctggcctgcc accagctgtg cgcccgaggg |
| 2041 | cactgctggg gtccagggcc cacccagtgt gtcaactgca gccagttcct tcggggccag |
| 2101 | gagtgcgtgg aggaatgccg agtactgcag gggctcccca gggagtatgt gaatgccagg |
| 2161 | cactgtttgc cgtgccaccc tgagtgtcag ccccagaatg gctcagtgac ctgttttgga |
| 2221 | ccggaggctg accagtgtgt ggcctgtgcc cactataagg accctccctt ctgcgtggcc |
| 2281 | cgctgcccca gcggtgtgaa acctgacctc tcctacatgc ccatctggaa gtttccagat |
| 2341 | gaggagggcg catgccagcc ttgccccatc aactgcaccc actcctgtgt ggacctggat |
| 2401 | gacaagggct gccccgccga gcagagagcc agccctctga cgtccatcat ctctgcggtg |
| 2461 | gttggcattc tgctggtcgt ggtcttgggg gtggtctttg ggatcctcat caagcgacgg |
| 2521 | cagcagaaga tccggaagta cacgatgcgg agactgctgc aggaaacgga gctggtggag |
| 2581 | ccgctgacac ctagcggagc gatgcccaac caggcgcaga tgcggatcct gaaagagacg |
| 2641 | gagctgagga aggtgaaggt gcttggatct ggcgcttttg gcacagtcta caagggcatc |
| 2701 | tggatccctg atggggagaa tgtgaaaatt ccagtggcca tcaaagtgtt gagggaaaac |
| 2761 | acatccccca aagccaacaa agaaatctta gacgaagcat acgtgatggc tggtgtgggc |
| 2821 | tccccatatg tctcccgcct tctgggcatc tgcctgacat ccacggtgca gctggtgaca |
| 2881 | cagcttatgc cctatggctg cctcttagac catgtccggg aaaaccgcgg acgcctgggc |
| 2941 | tcccaggacc tgctgaactg gtgtatgcag attgccaagg ggatgagcta cctggaggat |
| 3001 | gtgcggctcg tacacaggga cttggccgct cggaacgtgc tggtcaagag tcccaaccat |
| 3061 | gtcaaaatta cagacttcgg gctggctcgg ctgctggaca ttgacgagac agagtaccat |
| 3121 | gcagatgggg gcaaggtgcc catcaagtgg atggcgctgg agtccattct ccgccggcgg |
| 3181 | ttcacccacc agagtgatgt gtggagttat ggtgtgactg tgtgggagct gatgactttt |
| 3241 | ggggccaaac cttacgatgg gatcccagcc cgggagatcc ctgacctgct ggaaaagggg |
| 3301 | gagcggctgc cccagccccc catctgcacc attgatgtct acatgatcat ggtcaaatgt |
| 3361 | tggatgattg actctgaatg tcggccaaga ttccgggagt tggtgtctga attctcccgc |
| 3421 | atggccaggg acccccagcg ctttgtggtc atccagaatg aggacttggg cccagccagt |
| 3481 | cccttggaca gcaccttcta ccgctcactg ctggaggacg atgacatggg ggacctggtg |
| 3541 | gatgctgagg agtatctggt accccagcag ggcttcttct gtccagaccc tgccccgggc |
| 3601 | gctgggggca tggtccacca caggcaccgc agctcatcta ccaggagtgg cggtggggac |
| 3661 | ctgacactag ggctggagcc ctctgaagag gaggccccca ggtctccact ggcaccctcc |
| 3721 | gaaggggctg gctccgatgt atttgatggt gacctgggaa tgggggcagc caaggggctg |
| 3781 | caaagcctcc ccacacatga ccccagccct ctacagcggt acagtgagga ccccacagta |
| 3841 | cccctgccct ctgagactga tggctacgtt gcccccctga cctgcagccc ccagcctgaa |
| 3901 | tatgtgaacc agccagatgt tcggccccag cccccttcgc cccgagaggg ccctctgcct |
| 3961 | gctgcccgac ctgctggtgc cactctggaa aggcccaaga ctctctcccc agggaagaat |
| 4021 | ggggtcgtca aagacgtttt tgcctttggg ggtgccgtgg agaaccccga gtacttgaca |
| 4081 | ccccagggag gagctgcccc tcagccccac cctcctcctg ccttcagccc agccttcgac |
| 4141 | aacctctatt actgggacca ggacccacca gagcgggggg ctccacccag caccttcaaa |
| 4201 | gggacaccta cggcagagaa cccagagtac ctgggtctgg acgtgccagt gtgaaccaga |
| 4261 | aggccaagtc cgcagaagcc ctgatgtgtc ctcagggagc agggaaggcc tgacttctgc |
| 4321 | tggcatcaag aggtgggagg gccctccgac cacttccagg ggaacctgcc atgccaggaa |
| 4381 | cctgtcctaa ggaaccttcc ttcctgcttg agttcccaga tggctggaag gggtccagcc |
| 4441 | tcgttggaag aggaacagca ctggggagtc tttgtggatt ctgaggccct gcccaatgag |
| 4501 | actctagggt ccagtggatg ccacagccca gcttggccct ttccttccag atcctgggta |
| 4561 | ctgaaagcct tagggaagct ggcctgagag gggaagcggc cctaagggag tgtctaagaa |
| 4621 | caaaagcgac ccattcagag actgtccctg aaacctagta ctgcccccca tgaggaagga |
| 4681 | acagcaatgg tgtcagtatc caggctttgt acagagtgct tttctgttta gtttttactt |
| 4741 | tttttgtttt gtttttttaa agatgaaata aagacccagg gggagaatgg gtgttgtatg |
| 4801 | gggaggcaag tgtggggggt ccttctccac acccactttg tccatttgca aatatatttt |
| 4861 | ggaaaacagc taaaaaaaaa aaaaaaaaa |

Exemplary landmark residues, domains, and fragments of HER2 include, but are not limited to residues 57-4254 (coding region), residues 4767-4772 (polyA signal sequence), 4787 (polyA site), or 4872 (polyA site).

In other example, the peptide (epitope peptide) comprises a mammalian peptide, a viral peptide, or a bacterial peptide. For example, the peptide is selected from the group consisting of MASMTGGQQMG (SEQ ID NO: 4)—T7 peptide derived from the T7 major capsid protein; EQKLISEEDL (SEQ ID NO: 5)—Myc peptide derived from c-Myc; YPYDVPDYA (SEQ ID NO: 6)—hemagglutinin (HA) peptide derived from hemagglutinin; YTDIEMNRLGK (SEQ ID NO: 7)—vesiculovirus (VSV-G) peptide derived from the vesicular stomatitis viral glycoprotein; KETAAAKFERQHMDS (SEQ ID NO: 8)—S peptide derived from pancreatic ribonuclease A; GKPIPNPLLGLDST (SEQ ID NO: 9)—V5 peptide derived from the P and V proteins of the paramyxovirus of simian virus 5; DYKDDDDK (SEQ ID NO: 10) FLAG synthetic peptide; GAPVPYPDPLEPR (SEQ ID NO: 11)—E synthetic peptide; HHHHHH (SEQ ID NO: 12)—Histidine synthetic peptide; TKENPRSNQEESYDDNES (SEQ ID NO: 13)—NE-tag synthetic peptide; WSHPQFEK (SEQ ID NO: 14)—synthetic peptide recognized by streptavidin; PDRVRAVSHWSS (SEQ ID NO: 15)—peptide derived from the protein beta-catenin and optimized for higher affinity binding to the Spot-Tag Nanobody; and YTDIEMNRLGK (SEQ ID NO: 7)—vesicular stomatitis virus (VSV) synthetic peptide.

In some examples, the immune cell (e.g., a lymphocyte) recruiting molecule comprises a cytokine protein or a cytokine protein epitope. For example, the cytokine protein is less than 200 amino acids. In other examples, the cytokine protein is up to 350 amino acids. Exemplary cytokine protein epitopes include interleukin 2 (IL-2), interleukin 6 (IL-6), interleukin-7 (IL-7), interleukin-12 (IL-12), a tumor necrosis factor (TNF), or chemokines including α-chemokines or CXC chemokines, where CXC stands for Cys-X-Cys motif, and β-chemokines or CC chemokines, where CC stands for Cys-Cys motif near their amino terminus.

An exemplary chemokine comprises the human protein epitope C-X-C motif chemokine 10 (CXCL10) or Interferon gamma-induced protein 10 (IP-10) (UniProt P02778 or NP_001556.2, incorporated herein by reference), and an exemplary sequence comprises the amino acid sequence:

| (SEQ ID NO: 514) | |
|---|---|
| 1 | mnqtailicc lifltlsgiq gvplsrtvrc tcisisnqpv nprsleklei |
| 61 | ipasqfcprv eiiatmkkkg ekrclnpesk aiknllkavs kerskrsp |

Exemplary landmark residues, domains, and fragments of CXCL10 include, but are not limited to residues 1-21 (signal peptide), residues 22-98 (mature peptide). A fragment (e.g., a peptide or an epitope) of a CXCL10 protein is less than the length of the full length protein, e.g., a fragment is at least 3, 4, 5, 6, 7, 8, 9, 10, 20, 30, 40, 50, 100 or more residues in length, but less than e.g., 108 residues in the case of CXCL10 above.

The human CXCL10 nucleic acid sequence is provided below and is incorporated herein by reference (start and stop codons are underlined; NCBI GenBank: NM_001565.4).

| (SEQ ID NO: 517) | |
|---|---|
| 1 | gagacattcc tcaattgctt agacatattc tgagcctaca gcagaggaac ctccagtctc |
| 61 | agcaccatga atcaaactgc cattctgatt tgctgcctta tctttctgac tctaagtggc |
| 121 | attcaaggag tacctctctc tagaactgta cgctgtacct gcatcagcat tagtaatcaa |
| 181 | cctgttaatc caaggtcttt agaaaaactt gaaattattc ctgcaagcca attttgtcca |
| 241 | cgtgttgaga tcattgctac aatgaaaaag aagggtgaga agagatgtct gaatccagaa |
| 301 | tcgaaggcca tcaagaattt actgaaagca gttagcaagg aaaggtctaa aagatctcct |
| 361 | taaaaccaga ggggagcaaa atcgatgcag tgcttccaag gatggaccac acagaggctg |
| 421 | cctctcccat cacttcccta catggagtat atgtcaagcc ataattgttc ttagtttgca |
| 481 | gttacactaa aaggtgacca atgatggtca ccaaatcagc tgctactact cctgtaggaa |
| 541 | ggttaatgtt catcatccta agctattcag taataactct accctggcac tataatgtaa |
| 601 | gctctactga ggtgctatgt tcttagtgga tgttctgacc ctgcttcaaa tatttccctc |
| 661 | acctttccca tcttccaagg gtactaagga atctttctgc tttggggttt atcagaattc |
| 721 | tcagaatctc aaataactaa aaggtatgca atcaaatctg ctttttaaag aatgctcttt |
| 781 | acttcatgga cttccactgc catcctccca aggggcccaa attctttcag tggctaccta |
| 841 | catacaattc caaacacata caggaaggta gaaatatctg aaaatgtatg tgtaagtatt |
| 901 | cttatttaat gaaagactgt acaaagtaga agtcttagat gtatatattt cctatattgt |
| 961 | tttcagtgta catggaataa catgtaatta agtactatgt atcaatgagt aacaggaaaa |
| 1021 | ttttaaaaat acagatagat atatgctctg catgttacat aagataaatg tgctgaatgg |
| 1081 | ttttcaaaat aaaaatgagg tactctcctg gaaatattaa gaaagactat ctaaatgttg |
| 1141 | aaagatcaaa aggttaataa agtaattata actaa |

Exemplary landmark residues, domains, and fragments of CXCL1 include, but are not limited to residues 67-129 (signal peptide), residues 130-360 (mature peptide), or residues 67-363 (coding region).

The human amino acid sequence of CXCL9 is provided below and is incorporated herein by reference (NCBI GenBank NP_002407.1 or UniProt Q07325):

| (SEQ ID NO: 518) | |
|---|---|
| 1 | mkksqvlfll giillvligv qgtpvvrkgr cscistnqgt ihlqslkdlk qfapspscek |
| 61 | ieiiatlkng vqtclnpdsa dvkelikkwe kqvsqkkkqk ngkkhqkkkv lkvrksqrsr |
| 121 | qkktt |

Exemplary landmark residues, domains, and fragments of CXCL9 include, but are not limited to residues 1-22 (signal peptide) or residues 23-125 (mature peptide). A fragment (e.g., a peptide or an epitope) of a CXCL9 protein is less than the length of the full length protein, e.g., a fragment is at least 3, 4, 5, 6, 7, 8, 9, 10, 20, 30, 40, 50, 100 or more residues in length, but less than e.g., 125 residues in the case of CXCL9 above.

The human CXCL9 nucleic acid sequence is provided below and is incorporated herein by reference (start and stop codons are underlined; NCBI GenBank: NM_002416.3).

| SEQ ID NO: 519 | |
|---|---|
| 1 | aaagaatttc tcaggctcaa aatccaatac aggagtgact tggaactcca ttctatcact |
| 61 | atgaagaaaa gtggtgttct tttcctcttg ggcatcatct tgctggttct gattggagtg |
| 121 | caaggaaccc cagtagtgag aaagggtcgc tgttcctgca tcagcaccaa ccaagggact |
| 181 | atccacctac aatccttgaa agaccttaaa caatttgccc caagcccttc ctgcgagaaa |
| 241 | attgaaatca ttgctacact gaagaatgga gttcaaacat gtctaaaccc agattcagca |
| 301 | gatgtgaagg aactgattaa aaagtgggag aaacaggtca gccaaaagaa aaagcaaaag |
| 361 | aatgggaaaa aacatcaaaa aaagaaagtt ctgaaagttc gaaaatctca acgttctcgt |
| 421 | caaaagaaga ctacataaga gaccacttca ccaataagta ttctgtgtta aaaatgttct |
| 481 | attttaatta taccgctatc attccaaagg aggatggcat ataatacaaa ggcttattaa |
| 541 | tttgactaga aaatttaaaa cattactctg aaattgtaac taaagttaga aagttgattt |
| 601 | taagaatcca aacgttaaga attgttaaag gctatgattg tctttgttct tctaccaccc |
| 661 | accagttgaa tttcatcatg cttaaggcca tgattttagc aatacccatg tctacacaga |
| 721 | tgttcaccca accacatccc actcacaaca gctgcctgga agagcagccc taggcttcca |
| 781 | cgtactgcag cctccagaga gtatctgagg cacatgtcag caagtcctaa gcctgttagc |
| 841 | atgctggtga gccaagcagt ttgaaattga gctggacctc accaagctgc tgtggccatc |
| 901 | aacctctgta tttgaatcag cctacaggcc tcacacacaa tgtgtctgag agattcatgc |
| 961 | tgattgttat tgggtatcac cactggagat caccagtgtg tggctttcag agcctccttt |
| 1021 | ctggctttgg aagccatgtg attccatctt gcccgctcag gctgaccact ttatttcttt |
| 1081 | ttgttcccct ttgcttcatt caagtcagct cttctccatc ctaccacaat gcagtgcctt |
| 1141 | tcttctctcc agtgcacctg tcatatgctc tgatttatct gagtcaactc ctttctcatc |
| 1201 | ttgtccccaa caccccacag aagtgctttc ttctcccaat tcatcctcac tcagtccagc |
| 1261 | ttagttcaag tcctgcctct taaataaacc tttttggaca cacaaattat cttaaaactc |
| 1321 | ctgtttcact tggttcagta ccacatgggt gaacactcaa tggttaacta attcttgggt |
| 1381 | gtttatccta tctctccaac cagattgtca gctccttgag ggcaagagcc acagtatatt |
| 1441 | tccctgtttc ttccacagtg cctaataata ctgtggaact aggttttaat aattttttaa |
| 1501 | ttgatgttgt tatgggcagg atggcaacca gaccattgtc tcagagcagg tgctggctct |
| 1561 | ttcctggcta ctccatgttg gctagcctct ggtaacctct tacttattat cttcaggaca |
| 1621 | ctcactacag ggaccaggga tgatgcaaca tccttgtctt tttatgacag gatgtttgct |
| 1681 | cagcttctcc aacaataaga agcacgtggt aaaacacttg cggatattct ggactgtttt |
| 1741 | taaaaaatat acagtttacc gaaaatcata taatcttaca atgaaaagga ctttatagat |
| 1801 | cagccagtga ccaacctttt cccaaccata caaaaattcc ttttcccgaa ggaaaagggc |
| 1861 | tttctcaata agcctcagct ttctaagatc taacaagata gccaccgaga tccttatcga |
| 1921 | aactcatttt aggcaaatat gagttttatt gtccgtttac ttgtttcaga gtttgtattg |
| 1981 | tgattatcaa ttaccacacc atctcccatg aagaaaggga acggtgaagt actaagcgct |
| 2041 | agaggaagca gccaagtcgg ttagtggaag catgattggt gcccagttag cctctgcagg |
| 2101 | atgtggaaac ctccttccag gggaggttca gtgaattgtg taggagaggt tgtctgtggc |
| 2161 | cagaatttaa acctatactc actttcccaa attgaatcac tgctcacact gctgatgatt |
| 2221 | tagagtgctg tccggtggag atcccacccg aacgtcttat ctaatcatga aactccctag |
| 2281 | ttccttcatg taacttccct gaaaaatcta agtgtttcat aaatttgaga gtctgtgacc |
| 2341 | cacttacctt gcatctcaca ggtagacagt atataactaa caaccaaaga ctacatattg |
| 2401 | tcactgacac acacgttata atcatttatc atatatatac atacatgcat acactctcaa |
| 2461 | agcaaataat ttttcacttc aaaacagtat tgacttgtat accttgtaat ttgaaatatt |
| 2521 | ttctttgtta aaatagaatg gtatcaataa atagaccatt aatcagaaaa cagatcttga |
| 2581 | ttttttttct cttgaatgta cccttcaact gttgaatgtt taatagtaaa tcttatatgt |
| 2641 | ccttatttac tttttagctt tctctcaaat aaagtgtaac actagttgag ataacacatg |
| 2701 | aaagctcttt aaagggtcga tcgggaacag gaaaaaaaac ctatggaaaa tatgacaaca |
| 2761 | c |

Exemplary landmark residues, domains, and fragments of CXCL9 include, but are not limited to residues 61-126 (signal peptide) or residues 127-435 (mature peptide).

The human amino acid sequence of CXCL11 is provided below and is incorporated herein by reference (NCBI GenBank NP_005400.1 or UniProt 014625):

| (SEQ ID NO: 520) | |
|---|---|
| 1 | msvkgmaial avilcatvvq gfpmfkrgrc lcigpgvkav kvadiekasi mypsnncdki |
| 61 | eviitlkenk gqrclnpksk qarliikkve rknf |

Exemplary landmark residues, domains, and fragments of CXCL11 include, but are not limited to residues 1-21 (signal peptide), residues 22-94 (mature peptide), or residues 28-90 (chemokine-CXC domain). A fragment (e.g., a peptide or an epitope) of a CXCL11 protein is less than the length of the full length protein, e.g., a fragment is at least 3, 4, 5, 6, 7, 8, 9, 10, 20, 30, 40, 50, 75 or more residues in length, but less than e.g., 94 residues in the case of CXCL11 above.

The human CXCL11 nucleic acid sequence is provided below and is incorporated herein by reference (start and stop codons are underlined; NCBI GenBank: NM_005409.5).

| SEQ ID NO: 521 | |
|---|---|
| 1 | gttcagcatt tctactcctt ccaagaagag cagcaaagct gaagtagcag cagcagcacc |
| 61 | agcagcaaca gcaaaaaaca aacatgagtg tgaagggcat ggctatagcc ttggctgtga |
| 121 | tattgtgtgc tacagttgtt caaggcttcc ccatgttcaa aagaggacgc tgtctttgca |
| 181 | taggccctgg ggtaaaagca gtgaaagtgg cagatattga gaaagcctcc ataatgtacc |
| 241 | caagtaacaa ctgtgacaaa atagaagtga ttattaccct gaaagaaaat aaaggacaac |
| 301 | gatgcctaaa tcccaaatcg aagcaagcaa ggcttataat caaaaaagtt gaaagaaaga |
| 361 | atttttaaaa atatcaaaac atatgaagtc ctggaaaaga gcatctgaaa aacctagaac |
| 421 | aagtttaact gtgactactg aaatgacaag aattctacag taggaaactg agacttttct |
| 481 | atggttttgt gactttcaac ttttgtacag ttatgtgaag gatgaaaggt gggtgaaagg |
| 541 | accaaaaaca gaaatacagt cttcctgaat gaatgacaat cagaattcca ctgcccaaag |
| 601 | gagtccaaca attaaatgga tttctaggaa aagctacctt aagaaaggct ggttaccatc |
| 661 | ggagtttaca aagtgctttc acgttcttac ttgttgcatt atacattcat gcatttctag |
| 721 | gctagagaac cttctagatt tgatgcttac aactattctg ttgtgactat gagaacattt |
| 781 | ctgtctctag aagtcatctg tctgtattga tctttatgct atattactat ctgtggttac |
| 841 | ggtggagaca ttgacattat tactggagtc aagcccttat aagtcaaaag catctatgtg |
| 901 | tcgtaaaaca ttcctcaaac attttttcat gcaaatacac acttctttcc ccaaacatca |
| 961 | tgtagcacat caatatgtag ggagacattc ttatgcatca tttggtttgt tttataacca |
| 1021 | attcattaaa tgtaattcat aaaatgtact atgaaaaaaa ttatacgcta tgggatactg |
| 1081 | gcaaaagtgc acatatttca taaccaaatt agtagcacca gtcttaattt gatgtttttc |
| 1141 | aacttttatt cattgagatg ttttgaagca attaggatat gtgtgtttac tgtacttttt |
| 1201 | gttttgatcc gtttgtataa atgatagcaa tatcttggac acatctgaaa tacaaaatgt |
| 1261 | ttttgtctac caaagaaaaa tgttgaaaaa taagcaaatg tatacctagc aatcactttt |
| 1321 | actttttgta attctgtctc ttagaaaaat acataatcta atcaatttct ttgttcatgc |
| 1381 | ctatatactg taaaatttag gtatactcaa gactagttta aagaatcaaa gtcatttttt |
| 1441 | tctctaataa actaccacaa cctttctttt ttaaaaaaa |

Exemplary landmark residues, domains, and fragments of CXCL11 include, but are not limited to residues 84-146 (signal peptide), or residues 147-365 (mature peptide).

The human amino acid sequence of tumor necrosis factor (TNF) is provided below and is incorporated herein by reference (NCBI GenBank NP_000585.2 or (UniProt P01375):

| (SEQ ID NO: 522) | |
|---|---|
| 1 | mstesmirdv elaeealpkk tggpqgsrrc lflslfsfli vagattlfcl lhfgvigpqr |
| 61 | eefprdlsli splaqavrss srtpsdkpva hvvanpqaeg qlqwlnrran allangvelr |
| 121 | dnqlvvpseg lyliysqvlf kgqgcpsthv lithtisria vsyqtkvnll saikspcqre |
| 181 | tpegaeakpw yepiylggvf qlekgdrlsa einrpdyldf aesgqvyfgi ial |

Exemplary landmark residues, domains, and fragments of TNF include, but are not limited to residues 1-35 (cytoplasmic domain), residues 36-56 (helical transmembrane domain) and residues 57-233 (extracellular domain). A fragment (e.g., a peptide or an epitope) of a TNF protein is less than the length of the full length protein, e.g., a fragment is at least 3, 4, 5, 6, 7, 8, 9, 10, 20, 30, 40, 50, 100, 200 or more residues in length, but less than e.g., 233 residues in the case of TNF above.

The human TNF nucleic acid sequence is provided below and is incorporated herein by reference (start and stop codons are underlined; NCBI GenBank: NM_000594.4).

| SEQ ID NO: 523 | |
|---|---|
| 1 | agcagacgct ccctcagcaa ggacagcaga ggaccagcta agagggagag aagcaactac |
| 61 | agaccccccc tgaaaacaac cctcagacgc cacatcccct gacaagctgc caggcaggtt |
| 121 | ctcttcctct cacatactga cccacggctc caccctctct cccctggaaa ggacaccatg |
| 181 | agcactgaaa gcatgatccg ggacgtggag ctggccgagg aggcgctccc caagaagaca |
| 241 | ggggggcccc agggctccag gcggtgcttg ttcctcagcc tcttctcctt cctgatcgtg |
| 301 | gcaggcgcca ccacgctctt ctgcctgctg cactttggag tgatcggccc ccagagggaa |
| 361 | gagttcccca gggacctctc tctaatcagc cctctggccc aggcagtcag atcatcttct |
| 421 | cgaaccccga gtgacaagcc tgtagcccat gttgtagcaa accctcaagc tgaggggcag |
| 481 | ctccagtggc tgaaccgccg ggccaatgcc ctcctggcca atggcgtgga gctgagagat |
| 541 | aaccagctgg tggtgccatc agagggcctg tacctcatct actcccaggt cctcttcaag |
| 601 | ggccaaggct gcccctccac ccatgtgctc ctcacccaca ccatcagccg catcgccgtc |
| 661 | tcctaccaga ccaaggtcaa cctcctctct gccatcaaga gcccctgcca gagggagacc |
| 721 | ccagaggggg ctgaggccaa gccctggtat gagcccatct atctgggagg ggtcttccag |
| 781 | ctggagaagg gtgaccgact cagcgctgag atcaatcggc ccgactatct cgactttgcc |
| 841 | gagtctgggc aggtctactt tgggatcatt gccctgtgag gaggacgaac atccaacctt |
| 901 | cccaaacgcc tcccctgccc caatcccttt attaccccct ccttcagaca ccctcaacct |
| 961 | cttctggctc aaaaagagaa ttgggggctt agggtcggaa cccaagctta gaactttaag |
| 1021 | caacaagacc accacttcga aacctgggat tcaggaatgt gtggcctgca cagtgaagtg |
| 1081 | ctggcaacca ctaagaattc aaactggggc ctccagaact cactggggcc tacagctttg |
| 1141 | atccctgaca tctggaatct ggagaccagg gagcctttgg ttctggccag aatgctgcag |
| 1201 | gacttgagaa gacctcacct agaaattgac acaagtggac cttaggcctt cctctctcca |
| 1261 | gatgtttcca gacttccttg agacacggag cccagccctc cccatggagc cagctccctc |
| 1321 | tatttatgtt tgcacttgtg attatttatt atttatttat tatttattta tttacagatg |
| 1381 | aatgtattta tttgggagac cggggtatcc tgggggaccc aatgtaggag ctgccttggc |
| 1441 | tcagacatgt tttccgtgaa aacggagctg aacaataggc tgttcccatg tagccccctg |
| 1501 | gcctctgtgc cttcttttga ttatgttttt taaaatattt atctgattaa gttgtctaaa |
| 1561 | caatgctgat ttggtgacca actgtcactc attgctgagc ctctgctccc caggggagtt |
| 1621 | gtgtctgtaa tcgccctact attcagtggc gagaaataaa gtttgcttag aaaagaaa |

Exemplary landmark residues, domains, and fragments of TNF include, but are not limited to residues 280-285 (TNF domain), or residues 458-1678 (exon).

The human amino acid sequence of IL-2 is provided below and is incorporated herein by reference (NCBI GenBank NP_000577.1 or UniProt P60568)

| (SEQ ID NO: 487) | |
|---|---|
| 1 | myrmqllsci alslalvtns aptssstkkt qlqlehllld lqmilnginn yknpkltrml |
| 61 | tfkfympkka telkhlqcle eelkpleevl nlaqsknfhl rprdlisnin vivlelkgse |
| 121 | ttfmceyade tativeflnr witfcqsiis tlt |

Exemplary landmark residues, domains, and fragments of IL-2 include, but are not limited to residues 1-20 (signal peptide), residues 21-153 (mature peptide), or residue 23 (glycosylation site). A fragment (e.g., a peptide or an epitope) of a IL-2 protein is less than the length of the full length protein, e.g., a fragment is at least 3, 4, 5, 6, 7, 8, 9, 10, 20, 30, 40, 50, 100, 153 or more residues in length, but less than e.g., 153 residues in the case of IL-2 above.

The human IL-2 nucleic acid sequence is provided below and is incorporated herein by reference (start and stop codons are underlined; NCBI GenBank: NM_000586.4 (SEQ ID NO: 488).

| (SEQ ID NO: 488) | |
|---|---|
| 1 | ctatcaccta agtgtgggct aatgtaacaa agagggattt cacctacatc cattcagtca |
| 61 | gtctttgggg gtttaaagaa attccaaaga gtcatcagaa gaggaaaaat gaaggtaatg |
| 121 | ttttttcaga caggtaaagt ctttgaaaat atgtgtaata tgtaaaacat tttgacaccc |
| 181 | ccataatatt tttccagaat taacagtata aattgcatct cttgttcaag agttccctat |
| 241 | cactctcttt aatcactact cacagtaacc tcaactcctg ccacaatgta caggatgcaa |
| 301 | ctcctgtctt gcattgcact aagtcttgca cttgtcacaa acagtgcacc tacttcaagt |
| 361 | tctacaaaga aaacacagct acaactggag catttactgc tggatttaca gatgattttg |
| 421 | aatggaatta ataattacaa gaatcccaaa ctcaccagga tgctcacatt taagttttac |
| 481 | atgcccaaga aggccacaga actgaaacat cttcagtgtc tagaagaaga actcaaacct |
| 541 | ctggaggaag tgctaaattt agctcaaagc aaaaactttc acttaagacc cagggactta |
| 601 | atcagcaata tcaacgtaat agttctggaa ctaaagggat ctgaaacaac attcatgtgt |
| 661 | gaatatgctg atgagacagc aaccattgta gaatttctga acagatggat taccttttgt |
| 721 | caaagcatca tctcaacact gacttgataa ttaagtgctt cccacttaaa acatatcagg |
| 781 | ccttctattt atttaaatat ttaaatttta tatttattgt tgaatgtatg gtttgctacc |
| 841 | tattgtaact attattctta atcttaaaac tataaatatg gatcttttat gattcttttt |
| 901 | gtaagcccta ggggctctaa aatggtttca cttatttatc ccaaaatatt tattattatg |
| 961 | ttgaatgtta aatatagtat ctatgtagat tggttagtaa aactatttaa taaatttgat |
| 1021 | aaatataaa. |

Exemplary landmark residues, domains, and fragments of IL-2 include, but are not limited to residues 286-345 (signal peptide), residues 346-744 (mature peptide), or 286-747 (coding region).

The human amino acid sequence of IL-6 is provided below and is incorporated herein by reference (NCBI GenBank NP_000591.1 or UniProt P05231).

| (SEQ ID NO: 489) | |
|---|---|
| 1 | mnsfstsafg pvafslglll vlpaafpapv ppgedskdva aphrqpltss eridkqiryi |
| 61 | ldgisalrke tcnksnmces skealaennl nlpkmaekdg cfqsgfneet clvkiitgll |
| 121 | efevyleylq nrfesseeqa ravqmstkvl iqflqkkakn ldaittpdpt tnaslltklq |
| 181 | aqnqwlqdmt thlilrsfke flqsslralr qm |

Exemplary landmark residues, domains, and fragments of IL-6 include, but are not limited to residues 1-29 (signal peptide), residues 30-212 (mature peptide), or residue 73 (glycosylation site). A fragment (e.g., a peptide or an epitope) of a IL-6 protein is less than the length of the full length protein, e.g., a fragment is at least 3, 4, 5, 6, 7, 8, 9, 10, 20, 30, 40, 50, 100, 200 or more residues in length, but less than e.g., 212 residues in the case of IL-6 above.

The human IL-6 nucleic acid sequence is provided below and is incorporated herein by reference (start and stop codons are underlined; NCBI GenBank: NM_000600.5).

| (SEQ ID NO: 490) | |
|---|---|
| 1 | attctgccct cgagcccacc gggaacgaaa gagaagctct atctcccctc caggagccca |
| 61 | gctatgaact ccttctccac aagcgccttc ggtccagttg ccttctccct ggggctgctc |
| 121 | ctggtgttgc ctgctgcctt ccctgcccca gtacccccag gagaagattc caaagatgta |
| 181 | gccgccccac acagacagcc actcacctct tcagaacgaa ttgacaaaca aattcggtac |
| 241 | atcctcgacg gcatctcagc cctgagaaag gagacatgta acaagagtaa catgtgtgaa |
| 301 | agcagcaaag aggcactggc agaaaacaac ctgaaccttc caaagatggc tgaaaaagat |
| 361 | ggatgcttcc aatctggatt caatgaggag acttgcctgg tgaaaatcat cactggtctt |
| 421 | ttggagtttg aggtatacct agagtacctc cagaacagat ttgagagtag tgaggaacaa |
| 481 | gccagagctg tgcagatgag tacaaaagtc ctgatccagt tcctgcagaa aaaggcaaag |
| 541 | aatctagatg caataaccac ccctgaccca accacaaatg ccagcctgct gacgaagctg |
| 601 | caggcacaga accagtggct gcaggacatg acaactcatc tcattctgcg cagctttaag |
| 661 | gagttcctgc agtccagcct gagggctctt cggcaaatgt agcatgggca cctcagattg |
| 721 | ttgttgttaa tgggcattcc ttcttctggt cagaaacctg tccactgggc acagaactta |
| 781 | tgttgttctc tatggagaac taaaagtatg agcgttagga cactatttta attattttta |
| 841 | atttattaat atttaaatat gtgaagctga gttaatttat gtaagtcata tttatatttt |
| 901 | taagaagtac cacttgaaac attttatgta ttagttttga aataataatg gaaagtggct |
| 961 | atgcagtttg aatatccttt gtttcagagc cagatcattt cttggaaagt gtaggcttac |
| 1021 | ctcaaataaa tggctaactt atacatattt ttaaagaaat atttatattg tatttatata |
| 1081 | atgtataaat ggtttttata ccaataaatg gcattttaaa aaattca |

Exemplary landmark residues, domains, and fragments of IL-6 include, but are not limited to residues 64-150 (signal peptide), residues 151-699 (mature peptide), or 64-702 (coding region).

The human amino acid sequence of IL-7 is provided below and is incorporated herein by reference (NCBI GenBank NP_000871.1 or UniProt P13232)

| (SEQ ID NO: 491) | |
|---|---|
| 1 | mfhvsfryif glpplilvll pvassdcdie gkdgkqyesv lmvsidqlld smkeigsncl |
| 61 | nnefnffkrh icdankegmf lfraarklrq flkmnstgdf dlhllkvseg ttillnctgq |
| 121 | vkgrkpaalg eaqptkslee nkslkeqkkl ndlcflkrll qeiktcwnki lmgtkeh |

Exemplary landmark residues, domains, and fragments of IL-7 include, but are not limited to residues 1-25 (signal peptide), residues 26-177 (mature peptide), or residue 95 (glycosylation site). A fragment (e.g., a peptide or an epitope) of a IL-7 protein is less than the length of the full length protein, e.g., a fragment is at least 3, 4, 5, 6, 7, 8, 9, 10, 20, 30, 40, 50, 100, 150 or more residues in length, but less than e.g., 177 residues in the case of IL-7 above.

The human IL-7 nucleic acid sequence is provided below and is incorporated herein by reference (start and stop codons are underlined: NCHI GenBank: NM_000880.4)

| (SEQ ID NO: 492) | |
|---|---|
| 1 | acacttgtgg cttccgtgca cacattaaca actcatggtt ctagctccca gtcgccaagc |
| 61 | gttgccaagg cgttgagaga tcatctggga agtcttttac ccagaattgc tttgattcag |
| 121 | gccagctggt ttttcctgcg gtgattcgga aattcgcgaa ttcctctggt cctcatccag |
| 181 | gtgcgcggga agcaggtgcc caggagagag gggataatga agattccatg ctgatgatcc |
| 241 | caaagattga acctgcagac caagcgcaaa gtagaaactg aaagtacact gctggcggat |
| 301 | cctacggaag ttatggaaaa ggcaaagcgc agagccacgc cgtagtgtgt gccgcccccc |
| 361 | ttgggatgga tgaaactgca gtcgcggcgt gggtaagagg aaccagctgc agagatcacc |
| 421 | ctgcccaaca cagactcggc aactccgcgg aagaccaggg tcctgggagt gactatgggc |
| 481 | ggtgagagct tgctcctgct ccagttgcgg tcatcatgac tacgcccgcc tcccgcagac |
| 541 | catgttccat gtttctttta ggtatatctt tggacttcct cccctgatcc ttgttctgtt |
| 601 | gccagtagca tcatctgatt gtgatattga aggtaaagat ggcaaacaat atgagagtgt |
| 661 | tctaatggtc agcatcgatc aattattgga cagcatgaaa gaaattggta gcaattgcct |
| 721 | gaataatgaa tttaactttt ttaaaagaca tatctgtgat gctaataagg aaggtatgtt |
| 781 | tttattccgt gctgctcgca agttgaggca atttcttaaa atgaatagca ctggtgattt |
| 841 | tgatctccac ttattaaaag tttcagaagg cacaacaata ctgttgaact gcactggcca |
| 901 | ggttaaagga agaaaaccag ctgccctggg tgaagcccaa ccaacaaaga gtttggaaga |
| 961 | aaataaatct ttaaaggaac agaaaaaact gaatgacttg tgtttcctaa agagactatt |
| 1021 | acaagagata aaaacttgtt ggaataaaat tttgatgggc actaaagaac actgaaaaat |
| 1081 | atggagtggc aatatagaaa cacgaacttt agctgcatcc tccaagaatc tatctgctta |
| 1141 | tgcagttttt cagagtggaa tgcttcctag aagttactga atgcaccatg gtcaaaacgg |
| 1201 | attagggcat ttgagaaatg catattgtat tactagaaga tgaatacaaa caatggaaac |
| 1261 | tgaatgctcc agtcaacaaa ctatttctta tatatgtgaa catttatcaa tcagtataat |
| 1321 | tctgtactga tttttgtaag acaatccatg taaggtatca gttgcaataa tacttctcaa |
| 1381 | acctgtttaa atatttcaag acattaaatc tatgaagtat ataatggttt caaagattca |
| 1441 | aaattgacat tgctttactg tcaaaataat tttatggctc actatgaatc tattatactg |
| 1501 | tattaagagt gaaaattgtc ttcttctgtg ctggagatgt tttagagtta acaatgatat |
| 1561 | atggataatg ccggtgagaa taagagagtc ataaacctta agtaagcaac agcataacaa |
| 1621 | ggtccaagat acctaaaaga gatttcaaga gatttaatta atcatgaatg tgtaacacag |
| 1681 | tgccttcaat aaatggtata gcaaatgttt tgacatgaaa aaaggacaat ttcaaaaaaa |
| 1741 | taaaataaaa taaaaataaa ttcacctagt ctaaggatgc taaaccttag tactgagtta |
| 1801 | cattgtcatt tatatagatt ataacttgtc taaataagtt tgcaatttgg gagatatatt |
| 1861 | tttaagataa taatatatgt ttacctttta attaatgaaa tatctgtatt taattttgac |
| 1921 | actatatctg tatataaaat attttcatac agcattacaa attgcttact ttggaataca |
| 1981 | tttctccttt gataaaataa atgagctatg tattaa. |

Exemplary landmark residues, domains, and fragments of IL-7 include, but are not limited to residues 542-616 (signal peptide), residues 617-1072 (mature peptide), or 542-1075 (coding region).

The human amino acid sequence of IL-12 alpha subunit is provided below and is incorporated herein by reference (UniProt P29459).

| (SEQ ID NO: 493) | |
|---|---|
| 1 | mcparsllly atlvlldhls larnlpvatp dpgmfpclhh sqnllravsn mlqkarqtle |
| 61 | fypctseeid heditkdkts tveaclplel tknesclnsr etsfitngsc lasrktsfmm |
| 121 | alclssiyed lkmyqvefkt mnakllmdpk rqifldqnml avidelmqal nfnsetvpqk |
| 181 | ssleepdfyk tkiklcillh afriravtid rvmsylnas |

Exemplary landmark residues, domains, and fragments of L-12 alpha subunit include, but are not limited to residues 1-22 (signal peptide) or residues 23-219 (mature peptide). A fragment (e.g., a peptide or an epitope) of a IL-12 protein is less than the length of the full length protein, e.g., a fragment is at least 3, 4, 5, 6, 7, 8, 9, 10, 20, 30, 40, 50, 100, 200 or more residues in length, but less than e.g., 219 residues in the case of IL-12 above.

The human IL-12 sununit alpha nucleic acid sequence is provided below and is incorporated herein by reference (start and stop codons are underlined; NCBI GenBank: AF101062.1).

| (SEQ ID NO: 494) | |
|---|---|
| 1 | atgtggcccc ctgggtcagc ctcccagcca ccgccctcac ctgccgcggc cacaggtctg |
| 61 | catccagcgg ctcgccctgt gtccctgcag tgccggctca gcatgtgtcc agcgcgcagc |
| 121 | ctcctccttg tggctaccct ggtcctcctg gaccacctca gtttggccag aaacctcccc |
| 181 | gtggccactc cagacccagg aatgttccca tgccttcacc actcccaaaa cctgctgagg |
| 241 | gccgtcagca acatgctcca gaaggccaga caaactctag aattttaccc ttgcacttct |
| 301 | gaagagattg atcatgaaga tatcacaaaa gataaaacca gcacagtgga ggcctgttta |
| 361 | ccattggaat taaccaagaa tgagagttgc ctaaattcca gagagacctc tttcataact |
| 421 | aatgggagtt gcctggcctc cagaaagacc tcttttatga tggccctgtg ccttagtagt |
| 481 | atttatgaag acttgaagat gtaccaggtg gagttcaaga ccatgaatgc aaagcttctg |
| 541 | atggatccta agaggcagat ctttctagat caaaacatgc tggcagttat tgatgagctg |
| 601 | atgcaggccc tgaatttcaa cagtgagact gtgccacaaa aatcctccct tgaagaaccg |
| 661 | gatttttata aaactaaaat caagctctgc atacttcttc atgctttcag aattcgggca |
| 721 | gtgactattg atagagtgat gagctatctg aatgcttcct aa |

Exemplary landmark residues, domains, and fragments of IL-12 alpha subunit include, but are not limited to residues 1-762 (coding region), or residues 1-203 (interleukin binding region).

The human amino acid sequence of IL-12 beta subunit is provided below and is incorporated herein by reference (UniProt P29460).

| (SEQ ID NO: 524) | |
|---|---|
| 1 | mchgqlvisw fslvflaspl vaiwelkkdv yvveldwypd apgemvvltc dtpeedgitw |
| 61 | tldqssevlg sqktltiqvk efgdagqytc hkggevlshs llllhkkedg iwstdilkdq |
| 121 | kepknktflr ceaknysgrf tcwwlttist dltfsvkssr gssdpqgvtc gaatlsaerv |
| 181 | rgdnkeyeys vecqedsacp aaeeslpiev mvdavhklky enytssffir diikpdppkn |
| 241 | lqlkplknsr qvevsweypd twstphsyfs ltfcvqvqgk skrekkdrvf tdktsatvic |
| 301 | rknasisvra qdryysssws ewasvpcs |

Exemplary landmark residues, domains, and fragments of IL-12 beta subunit include, but are not limited to residues 1-22 (signal peptide) or residues 23-328 (mature peptide). A fragment (e.g., a peptide or an epitope) of a IL-12 beta subunit protein is less than the length of the full length protein, e.g., a fragment is at least 3, 4, 5, 6, 7, 8, 9, 10, 20, 30, 40, 50, 100, 200 or more residues in length, but less than e.g., 328 residues in the case of IL-12 beta subunit above.

The human IL-12 subunit beta nucleic acid sequence is provided below and is incorporated herein by reference (stop and start codons are bold and underlined NM_002187.3).

| SEQ ID NO: 525 | |
|---|---|
| 1 | agaagaaaca acatctgttt cagggccatt ggactctccg tcctgcccag agcaagatgt |
| 61 | gtcaccagca gttggtcatc tcttggtttt ccctggtttt tctggcatct cccctcgtgg |
| 121 | ccatatggga actgaagaaa gatgtttatg tcgtagaatt ggattggtat ccggatgccc |
| 181 | ctggagaaat ggtggtcctc acctgtgaca cccctgaaga agatggtatc acctggacct |
| 241 | tggaccagag cagtgaggtc ttaggctctg gcaaaaccct gaccatccaa gtcaaagagt |
| 301 | ttggagatgc tggccagtac acctgtcaca aaggaggcga ggttctaagc cattcgctcc |
| 361 | tgctgcttca caaaaaggaa gatggaattt ggtccactga tattttaaag gaccagaaag |
| 421 | aacccaaaaa taagaccttt ctaagatgcg aggccaagaa ttattctgga cgtttcacct |
| 481 | gctggtggct gacgacaatc agtactgatt tgacattcag tgtcaaaagc agcagaggct |
| 541 | cttctgaccc ccaaggggtg acgtgcggag ctgctacact ctctgcagag agagtcagag |
| 601 | gggacaacaa ggagtatgag tactcagtgg agtgccagga ggacagtgcc tgcccagctg |
| 661 | ctgaggagag tctgcccatt gaggtcatgg tggatgccgt tcacaagctc aagtatgaaa |
| 721 | actacaccag cagcttcttc atcagggaca tcatcaaacc tgacccaccc aagaacttgc |
| 781 | agctgaagcc attaaagaat tctcggcagg tggaggtcag ctgggagtac cctgacacct |
| 841 | ggagtactcc acattcctac ttctccctga cattctgcgt tcaggtccag ggcaagagca |
| 901 | agagagaaaa gaaagataga gtcttcacgg acaagacctc agccacggtc atctgccgca |
| 961 | aaaatgccag cattagcgtg cgggcccagg accgctacta tagctcatct tggagcgaat |
| 1021 | gggcatctgt gccctgcagt taggttctga tccaggatga aaatttggag gaaaagtgga |
| 1081 | agatattaag caaaatgttt aaagacacaa cggaatagac ccaaaaagat aatttctatc |
| 1141 | tgatttgctt taaaacgttt ttttaggatc acaatgatat ctttgctgta tttgtatagt |
| 1201 | tagatgctaa atgctcattg aaacaatcag ctaatttatg tatagatttt ccagctctca |
| 1261 | agttgccatg ggccttcatg ctatttaaat atttaagtaa tttatgtatt tattagtata |
| 1321 | ttactgttat ttaacgtttg tctgccagga tgtatggaat gtttcatact cttatgacct |
| 1381 | gatccatcag gatcagtccc tattatgcaa aatgtgaatt taattttatt tgtactgaca |
| 1441 | acttttcaag caaggctgca agtacatcag ttttatgaca atcaggaaga atgcagtgtt |
| 1501 | ctgataccag tgccatcata cacttgtgat ggatgggaac gcaagagata cttacatgga |
| 1561 | aacctgacaa tgcaaacctg ttgagaagat ccaggagaac aagatgctag ttcccatgtc |
| 1621 | tgtgaagact tcctggagat ggtgttgata aagcaattta gggccactta cacttctaag |
| 1681 | caagtttaat ctttggatgc ctgaatttta aaagggctag aaaaaaatga ttgaccagcc |
| 1741 | tgggaaacat aacaagaccc cgtctctaca aaaaaaattt aaaattagcc aggcgtggtg |
| 1801 | gctcatgctt gtggtcccag ctgttcagga ggatgaggca ggaggatctc ttgagcccag |
| 1861 | gaggtcaagg ctatggtgag ccgtgattgt gccactgcat accagcctag gtgacagaat |
| 1921 | gagaccctgt ctcaaaaaaa aaaatgattg aaattaaaat tcagctttag cttccatggc |
| 1981 | agtcctcacc cccacctctc taaaagacac aggaggatga cacagaaaca ccgtaagtgt |
| 2041 | ctggaaggca aaaagatctt aagattcaag agagaggaca agtagttatg gctaaggaca |
| 2101 | tgaaattgtc agaatggcag gtggcttctt aacagccctg tgagaagcag acagatgcaa |
| 2161 | agaaaatctg gaatcccttt ctcattagca tgaatgaacc tgatacacaa ttatgaccag |
| 2221 | aaaatatggc tccatgaagg tgctactttt aagtaatgta tgtgcgctct gtaaagtgat |
| 2281 | tacatttgtt tcctgtttgt ttatttattt atttattttt gcattctgag gctgaactaa |
| 2341 | taaaaactct tctttgtaat cata |

Exemplary landmark residues, domains, and fragments of IL-12 beta subunit include, but are not limited to residues 57-122 (signal peptide), or residues 123-1040 (mature protein).

In other aspects of the invention, the methods described herein comprise administering a pHLIP® construct comprising an antibody recruiting molecule that is linked to one or more pHLIP® peptides. For example, the antibody recruiting molecule comprises an epitope, including for example a small molecule (alternatively, a “small molecule antigen”). For example, the small molecule epitope comprises a Di-nitrophenyl (DNP) group, or a derivative thereof. As used herein, the terms “DNP” and “2,4-DNP” interchangeably refer to 2,4-dinitrophenol, a salt, solvate or adduct thereof.

In examples, the small molecule epitope comprises 0-(2,4-dinitrophenyl)hydroxylamine (structure provided below):

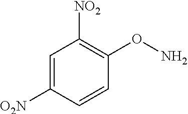 [SMILES: NOc1ccc([N+](=O)[O-])cc1[N+](=O)[O-]]

In embodiments, the composition of the present disclosure includes modified DNPs, such as those conjugated to polyethylene glycol may be used in the present disclosure. The DNP, DNP derivative, or salt in the composition of the present disclosure may have a molecular weight in the range from 200 Da to 1,000 Da or from 200 Da to 500 Da.

In some examples, the DNP derivative may be substituted, e.g., with an alkyl group, an alkylene group, a heteroalkyl group, a cycloalkyl group, an aryl group, or any combination thereof.

Also within the invention is a composition comprising an antibody or immune cell recruiting molecule linked to one or more pHLIP® peptides by a non-cleavable linker compound for eliciting an anti-tumor response in a subject. Also provided herein are methods for promoting an immune response. For example, the method comprises administering a composition comprises an epitope comprising at least 4 amino acids conjugated to a pHLIP® peptide. The pHLIP® peptide positions an epitope on the surface of the targeted cells in diseased tissue to induce an immune response predominantly in diseased tissue. For example, if the epitope is a peptide, it can be added as an extension of the non-inserting end of the pHLIP peptide. Furthermore, the epitope then interacts with endogenous or exogenous immune cells, endogenous antibodies and proteins, e.g., pre-existing antibodies and proteins in the subject's body, or exogenous antibodies administrated into body, or antibodies generated in the course of vaccination, which then induce an immune response. Also provided herein are methods for promoting delivery of cytotoxic payloads with ADCs to induce cell killing. Furthermore, the epitope interacts with ADCs administrated into body, which then induce cell killing.

Furthermore, provided herein are methods of treating a diseased tissue with a naturally acidic extracellular environment or a tissue with an artificially induced acidic extracellular environment relative to normal physiological pH in a subject. For example, the diseased tissue includes a cancerous tissue or a tumor. As described above, the composition recruits the subject's immune cells, endogenous antibodies and proteins to induce an immune response, and thereby treats the diseased tissue in the subject. The immune response can include, for example, initiation of complement-dependent cytotoxicity (CDC), antibody-dependent cell-mediated cytotoxicity (ADCC), or the release of cytokines or inflammatory mediators to promote T-cell or NK-cell responses. The immune response can include, for example, homing of T-cells and NK-cells and their activation. Also as described above, the composition promotes binding of antibody-drug conjugates to the cells in targeted tissue, and thus promotes cell killing.

Also within the invention is a method of augmenting (increasing) an immune response in a subject, comprising administering to the subject a composition comprising an epitope linked to a pHLIP® peptide as described above. In some examples, the composition is administered using methods well known in the art, e.g., the composition is injected directly into a tumor mass. Alternatively, the composition is systemically administered.

Formulations provided herein may include an Epitope-Linker-Peptide, where “Peptide” is a pHLIP® peptide. The formulation may be suitable for intravenous, subcutaneous, intraarterial, intraperitoneal, intracerebral, intracerebroventricular, intrathecal, intracardiac, intracavernous, intraosseous, intraocular, or intravitreal administration are also provided. In some examples, a formulation is used for intramuscular, intradermal, transdermal, transmucosal, intralesional, subcutaneous, topical, epicutaneous, extra-amniotic, intravaginal, intravesical, nasal, or oral administration. The present subject matter also includes a formulation for intravesical instillation. In some embodiments, the formulation is used for the treatment of cancer (e.g., solid tumors).

Also provided herein is a formulation comprising an Epitope-Linker-Peptide that comprises multiple pHLIP® peptides, e.g., for systemic administration. Also provided herein is a formulation comprising an Epitope-Linker-Peptide that comprises multiple epitopes, e.g., for systemic administration. In certain embodiments, the formulation is used for the treatment of cancer or diseases associated with inflammation.

Provided herein is a method of treating cancer or inflammation in a subject, comprising administering to the subject an effective amount of a pH-triggered compound (a pHLIP® peptide) linked to an epitope (a “pHLIP® construct”), which is then delivered by pHLIP® to the surface of the cell. For example, the cancer includes a solid tumor. Non-limiting examples of cancer include colon cancer, prostate cancer, breast cancer, bladder cancer, lung cancer, skin cancer, liver cancer, bone cancer, ovarian cancer, stomach cancer, pancreatic cancer, testicular cancer, and brain cancer.

Systemic or blood-borne tumor cells, e.g., cancers of the circulatory system, may also be treated using the pHLIP® peptide constructs.

The composition preferentially targets a diseased tissue compared to a healthy tissue, thereby minimizing damage to the healthy tissue. For example, the composition selectively promotes cell killing in the diseased tissue, e.g., the tumor cell.

Included herein are pharmaceutical compositions comprising a pH-triggered peptide linked to an epitope and a pharmaceutically acceptable carrier.

As used herein, “effective” when referring to an amount of a compound refers to the quantity of the compound that is sufficient to yield a desired response without undue adverse side effects (such as toxicity, irritation, or allergic response) commensurate with a reasonable benefit/risk ratio when used in the manner of this disclosure.

In some embodiments, a subject is a mammal. In certain embodiments, the mammal is a rodent (e.g., a mouse or a rat), a primate (e.g., a chimpanzee, a gorilla, a monkey, a gibbon, a baboon), a cow, a camel, a dog, a cat, a horse, a llama, a sheep, a goat, or a pig. In preferred embodiments, the subject is a human.

In some examples, the exogenous cells or monoclonal antibody or antibody-drug conjugate is administered to the subject during or after administration of the epitope pHLIP® peptide construct. In some examples, the subject comprises endogenous, e.g., pre-existing, antibodies or immune cells to the epitope of the pHLIP® peptide construct. Alternatively, the subject does not comprise existing antibodies to the epitope delivered by the construct (at the time of initial administration). In such examples, a subject may be immunized with the epitope to generate an antibody response and then the subject is subsequently administered the pHLIP®-epitope construct. In such examples, the construct is administered after administration of the antigen epitope in a form that is not conjugated to a pHLIP®.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram of a pHLIP® construct in which an epitope is linked to a pHLIP® peptide.

FIG. 2A is a diagram of a pHLIP® construct in which multiple epitopes are linked to a single pHLIP® peptide.

FIG. 2B is a diagram of pHLIP® construct in which two epitopes are linked to a single pHLIP® peptide.

FIG. 3 is a diagram of a pHLIP® construct in which an epitope is linked to multiple pHLIP® peptides.

FIG. 4 is a diagram of a pHLIP® construct in which multiple epitopes are linked to multiple pHLIP® peptides.

FIG. 5A is a schematic presentation of epitope tethered to the surface of cell by pHLIP®. As a result, the targeted cell becomes decorated with epitopes and endogenous (natural) antibodies, exogenous antibodies, ADCs administrated into body, antibodies which are produced (generated) in the course of vaccination, and bind the targeted cell to promote cell killing.

FIG. 5B is a schematic presentation of two epitopes tethered to the surface of cell by pHLIP® to bind two heads of one antibody molecule. As a result, the targeted cell becomes decorated with epitopes and endogenous (natural) antibodies, or exogenous antibodies, or ADCs administrated into body, or antibodies, which are produced (generated) in the course of vaccination bind targeted cell to promote cell killing.

FIG. 6 depict fluorescent (647 nm) images obtained on tumor spheroids treated with an anti-DNP fluorescent antibody, DAPI, or an anti-DNP fluorescent antibody pre-mixed with DNP-PEG12-pHLIP® construct followed by washing and imaging (upper raw), and treated with DNP-pHLIP®, DNP-PEG4-pHLIP® D or DNP-PEG12-pHLIP® first, washed, and then treated with anti-DNP fluorescent antibody followed by washing and imaging of spheroids (bottom raw).

FIG. 7 depict fluorescent images obtained on tumor spheroids treated with DNP-PEG12-pHLIP® first, washed, and then treated with anti-DNP fluorescent antibody and DAPI, washing and imaging of tumor spheroids at different channels using an inverted confocal microscope and 40× objective.

FIG. 8 depicts a representative overlay of fluorescent and brightfield images of tumor spheroids treated with DNP-PEG12-pHLIP®, washed, then treated with anti-DNP antibody or IgM followed by addition of human serum with active complement and PI fluorescent dye, followed by washing and imaging. The images were obtained 2 hours after the treatment.

### DETAILED DESCRIPTION

The invention features compositions and methods for decorating target cells (cancer cells) using epitope-pHLIP® compounds, such that the pHLIP® targets tumors by responding to cell surface acidity, inserting into cancer cell membranes, and locating (positioning) a specified epitope on the cell surface to induce cell killing.

Epitopes bind and/or attract exogenous immune cells (T-cells or NK-cells) or exogenously developed monoclonal antibodies or antibody-drug conjugates administrated into body during or after of epitope-pHLIP® compound administration, and selectively promote cell killing while sparing normal tissues. Among these epitopes include epitopes for developed monoclonal antibodies.

Epitopes bind and/or attract endogenous (natural) immune cells (lymphocytes) or endogenous antibodies already present in the blood, and selectively initiate a specific immune response to attack the tumor while sparing normal tissues. Among these epitopes are epitopes associated with the surfaces of animal cells and bacteria. In many cases, humans already have developed antibodies in blood stream, which recognize such epitopes. Epitopes delivered according to the methods described herein may also bind/attract endogenous antibodies, which are produced (generated) in the course of vaccination. The epitopes (any antigen molecule used for vaccination), is linked (conjugated) to a pHLIP® peptide and delivered to tumors to promote an immune reaction within target tissue, e.g., by using harnessing influenza vaccines that create antibodies to virus surface epitopes. In some examples, an individual is immunized using a chosen antigen, followed by targeting the antigen targeted to tumor cells by a pHLIP® peptide.

### Decorated Acidic Diseased Cells

Acidic diseased cells, e.g., cancer cells are targeted using epitope-pHLIP® peptide compositions, such that the pHLIP® peptide targets tumors by responding to cell surface acidity, inserting into cancer cell membranes, and locating/positioning a specific epitope on the cell surface to induce and promote cellular responses including immune stimulation and inhibition of cell proliferation. The immune stimulation leads to cytotoxicity and death of the acidic disease cell.

### Epitope-pHLIP® Peptide Compositions

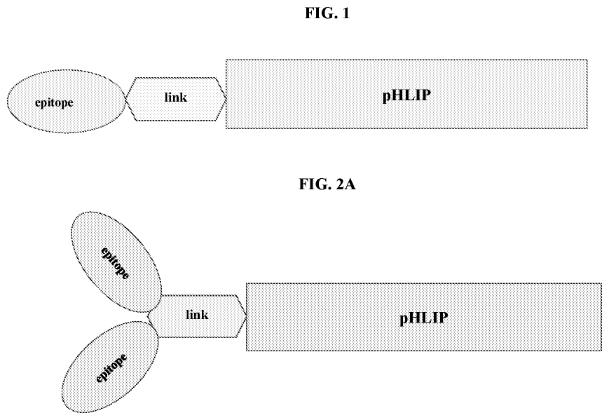

Representations of exemplary pHLIP® compositions for therapeutic use are shown in FIGS. 1-4. The compositions comprise a pHLIP® peptide (or pHLIP® conjugate, where the pHLIP® peptide is linked to an epitope for delivery/positioning on the surface of diseased cells). pHLIP® peptides are described here and in U.S. Pat. Nos. 9,814,781 and 9,289,508 (hereby incorporated by reference) as well as U.S. Patent Publication 20180117183, 20180064648, 20180221500, 20180117183, 20180064648, 20160256560, 20150191508, 20150051153, and 20120142042, 20120039990, 20080233107, as well as PCT Application No. PCT/US2017/023458 (cyclic pHLIP® peptides), each of which is hereby incorporated by reference.

FIG. 1 shows a construct in which an epitope is linked to a pHLIP® peptide.

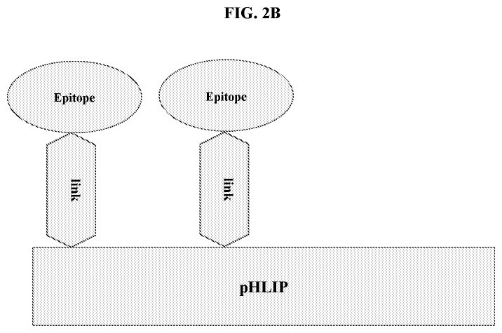

FIGS. 2A and 2B show a construct in which multiple epitopes are linked to a single pHLIP® peptide.

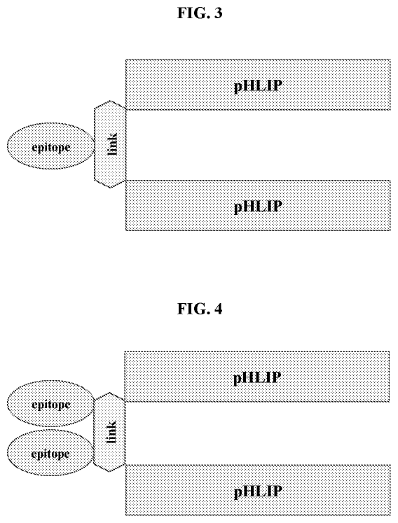

FIG. 3 shows a construct in which an epitope is linked to multiple pHLIP® peptides.

FIG. 4 shows a construct in which multiple epitopes are linked to multiple pHLIP® peptides.

An exemplary composition comprises the following formula:

Epitope-Linker-Peptide

The “Epitope” may include a peptide, a protein or a fragment thereof, or a small molecule such as an organic molecule, to induce an immune response or promote cell killing by attracting endogenous (pre-existing) immune cells or antibodies, exogenous (administered as in passive antibody-based immunotherapy) engineered immune cells or purified antibodies, ADCs administrated into humans, or antibodies generated in the course of vaccination.

“Peptide” is a pHLIP® peptide (a non-limiting example is a pHLIP® peptide comprising the sequence AXDDQNPWRAYLDLLFPTDTLLLDLLWA (SEQ ID NO: 18) or AXDQDNPWRAYLDLLFPTDTLLLDLLWA (SEQ ID NO: 495), where “X” is a functional group, e.g., for conjugation purposes, selected from a lysine (Lys), a cysteine (Cys), or an Azido-containing amino acid. In some cases “Peptide” is a pHLIP® conjugate, where the pHLIP® peptide is linked with a drug molecule for intracellular delivery.

“Linker” comprises a covalent bond, or a chemical linker, or an extension of the membrane non-inserting flanking region of pHLIP® peptide. If the epitope is a peptide or protein, it may be constructed as an extension of the pHLIP® peptide, and no linker may be required. Non-limiting example of linker is a polyethylene glycol (PEG) polymer in size from 200 Da to 20 kDa. Non-limiting example of an extension is a poly-Glycine polypeptide. Epitope(s) are also linked to pHLIP® peptide(s) via non-cleavable link(s).

Non-limiting examples of a Linker is a mucin domain, which is a high molecular weight, heavily glycosylated protein (glycoconjugate) produced by epithelial tissues in most humans. In other examples, the linker may include biopolymers, including, for example cellulose, starch or chitin.

An exemplary composition comprises the following formula:

Epitope2-Linker2-Pept

The “Epitope” comprises a peptide, a protein or a fragment thereof, or a small molecule such as an organic molecule, to induce an immune response or promote cell killing by attracting endogenous (pre-existing) immune cells or antibodies, exogenous (administered as in passive antibody-based immunotherapy) engineered T-cells or NK-cells or purified antibodies, ADCs administrated into humans, or antibodies generated as a result of vaccination.

“Peptide” is a pHLIP® peptide comprising the sequence: AX(Z)nXPWRAYLDLLFPTDTLLLDLLWA (SEQ ID NO: 496), wherein upper case “X” indicates any amino acid residue, and can include a lysine (Lys), a cysteine (Cys), or an Azido-containing amino acid. The X may be used for conjugation to another moiety. The “Z” indicates any amino acid residue, and n can be any integer between and including 1-10 (e.g., 1≤n≤10). For example, (Z)n may be QDNDQN (SEQ ID NO: 497) or any combination of polar amino acid residues including D, E, N or Q.

A compound is characterized as polar if it has a log P of less than −0.4. The epitope compound may be moderately hydrophobic. Polar: Log P<−0.4; Moderately hydrophobic: 2.5<Log P<−0.4; and Hydrophobic: Log P>2.5. The polarity and/or hydrophobicity of an epitope is measured using methods known in the art, e.g., by determining Log P, in which P is the octanol-water partition coefficient. A substance is dissolved into an octanol-water mixture, mixed, and allowed to come to equilibration. The amount of substance in each (or one) phases is then measured. The measurements itself could be in a number of ways known in the art, e.g., by measuring absorbance, or determining the amount using NMR, HPLC, or other known methods. As described herein, moderately hydrophobic, for example, is defined as molecule with Log P value in the range of 2.5 to −0.4, there are a lot of examples.

Linker is a linker, wherein the linker is a polyethylene glycol, PEG For example, the formula PEGm, includes that “m” may be any integer between and including 12 and 24 (e.g., 12≤m≤SPEG12-24 polymer). Each “—” may be a covalent bond.

When an epitope is conjugated to the pHLIP® peptide via a PEG12-24 linker and 6-8 residues are positioned between epitope-PEG attachments to the pHLIP® peptide, the distance between epitopes can be in the range of 5-25 nm. Alternatively, the distance may be about 10 nm, or 10-15 nm, which corresponds to a typical distance between the two antigen binding sites binding sites of an antibody.

The pHLIP® compositions described are used for decoration of cells in targeted acidic tissue to elicit an immune response or promote delivery of cytotoxic payload by ADCs to kill the diseased cells or tissue.

Non-limiting examples of epitopes are the following:

### Peptide Epitope-pHLIP® Peptide Compositions

Peptide antigens, e.g., peptides less than 50 amino acid residues in length, e.g., peptide antigens greater than 5 and less than 20, less than 15, less than 10, or less than 8 amino acid residues.

HER2 (Also Referred to as Receptor Tyrosine-Protein Kinase erbB-2 or Human Epidermal Growth Factor 2)

The HER family of transmembrane tyrosine kinase receptors are composed of four members, BER1 to HER4. HER2 is a ligand-orphan receptor expressed in many human tumors and overexpressed in 25-30% of breast cancers. HER2 amplifies the signal provided by other receptors of the HER family by forming heterodimers. The role of HER2 in the HER signaling network led to the development of anti-HER2 monoclonal antibodies (MAbs) for cancer therapy. In particular, the humanized MAb trastuzumab (Herceptin) or Herceptin-drug conjugates have antitumor activity against HER2-overexpressing human breast tumor cells and are widely used for the treatment of women with HER2 overexpressing breast cancers. One of the primary effects of trastuzumab is to induce antibody-dependent cellular cytotoxicity or promote cell killing by use of ADCs. In addition, trastuzumab induces HER2 receptor down modulation, inhibits critical signaling pathways (i.e. ras-Raf-MAPK and PI3K/Akt), and blocks cell cycle progression by inducing the formation of p27/Cdk2 complexes, inhibits HER2 cleavage, preceding antibody-induced receptor down modulation, which may also contribute to its antitumor activity in some cancers. A limitation of trastuzumab is that its activity is largely restricted to breast cancers with the highest level of HER2 overexpression.

As described above, humanized antibodies against the HER2 receptor have been developed for the treatment of HER2-positive breast cancer. However, not all breast tumors are HER2-positive, some of them are HER2 negative and in some cases during treatment, HER2-positive cancers can transform into HER2 negative cancers, and then treatment is not effective. Also, many other tumors are HER2 negative. The constructs/conjugates described herein decorate all cancer cells (regardless of tissue of origin) with HER2 epitopes to enhance antibody therapy. Useful epitopes include a small peptide mimic of the receptor binding site or a protein-pHLIP® peptide fusion construct, where the protein resembles the entire extracellular domain of a receptor or part of it.

Non-limiting examples of HER2 mimicking peptides, which show high affinity binding to anti-HER-2 monoclonal antibody trastuzumab (Herceptin) include the following peptides:

(SEQ ID NO: 1)
QVSHWVSGLAEGSFG
(SEQ ID NO: 2)
LSHTSGRVEGSVSLL
(SEQ ID NO: 3)
QMWAPQWGPD

The constructs are useful to decorate all cancer cells with HER2 epitopes to enhance antibody therapy. A useful epitope comprises a small peptide mimic of the receptor binding site or a protein-pHLIP® fusion construct, where the protein resembles the entire extracellular domain of a receptor or part of it. An additional strategy includes use a pHLIP® peptide to position epitopes for therapy using ADCs, some of which have been approved for clinical uses including trastuzumab emtansine, an anti-HER2 antibody conjugated with cytotoxic maytansinois payload (DM1 or mertansine), which is a potent tubulin inhibitor. By targeting the epitopes already in use, or by developing ADCs for new epitopes, the effective use of ADCs is significantly expanded. Other monoclonal antibodies developed for cancer treatment

In addition to anti-HER2 antibodies the non-limiting examples of monoclonal antibodies directed against different receptors currently in clinical use for cancer treatment include daratumumab (binds to CD38—cluster of differentiation 38); Ainutuximab (binds to glycolipid GD2—disialoganglioside); bevacizumab (binds to VEGF-A—vascular endothelial growth factor A); cetuximab, necitumumab and panitumumab (bind to EGFR—epidermal growth factor), elotuzumab (binds to CD 319—cluster of differentiation 319), necitumumab (binds EGFR), ramucirumab (binds to VEGFR2). Epitope-pHLIP® constructs could be developed to use with all of these antibodies and their drug conjugates.

### Other Examples of Peptide Epitopes

Non-limiting examples of peptide epitopes for conjugation to pHLIP® peptides for which monoclonal antibodies are already developed include:

(SEQ ID NO: 4): a T7 peptide derived from the T7
major capsid protein;
MASMTGGQQMG
(SEQ ID NO: 5): a Myc peptide derived from c-Myc;
EQKLISEEDL
(SEQ ID NO: 6): a hemagglutinin (HA) peptide
derived from hemagglutinin;
YPYDVPDYA
(SEQ ID NO: 7): a vesiculovirus (VSV-G) peptide
derived from the vesicular stomatitis viral
glycoprotein;
YTDIEMNRLGK
(SEQ ID NO: 8): an S peptide derived from
pancreatic ribonuclease A;
KETAAAKFERQHMDS
(SEQ ID NO: 9): a V5 peptide derived from the P
and V proteins of the paramyxovirus of simian
virus 5;
GKPIPNPLLGLDST
(SEQ ID NO: 15): a peptide derived from the
protein beta-catenin and optimized for higher
affinity binding to the Spot-Tag Nanobody;
PDRVRAVSHWSS
(SEQ ID NO: 10): a FLAG synthetic peptide;
DYKDDDDK
(SEQ ID NO: 11): an E synthetic peptide;
GAPVPYPDPLEPR
(SEQ ID NO: 12): a Histidine synthetic peptide;
HHHHHH
(SEQ ID NO: 13): an NE synthetic peptide;
TKENPRSNQEESYDDNES
(SEQ ID NO: 14): a synthetic peptide recognized by
streptavidin;
WSHPQFEK
(SEQ ID NO: 7): a vesicular stomatitis virus (VSV)
synthetic peptide.
YTDIEMNRLGK

Protein Epitope pHLIP® Peptide Compositions

In addition to peptide epitopes, larger epitope-containing cytokine proteins may be used for the production of the compositions described. Cytokines are small proteins from 5 to 20 kDa, which perform immuno-modulating role. Among cytokine proteins are IL-17, TNF, CXCL and CCL chemokines.

For example, CXCL9, CXCL10 or CXCL11 chemokine is expressed as N-terminal part of pHLIP® to induce homing and activation of T-cells and NK-cells when pHLIP® inserts into cellular membrane and tethers CXCL9 or CXCL10 or CXCL11 proteins to surface of membrane.

The cytokine can be expressed together with a mucin-domain (a glycosylated protein) as a linker (spacer) between cytokine and pHLIP® peptide to allow the cytokine to be fully exposed to the extracellular space.

The nucleic acid sequence encoding CXCL10 chemokine and the amino acid sequence of the protein antigen is described below.

SEQ ID NO: 498
ccagtctcagcaccatgaatcaaactgccattctgatttgctgccttatct
ttctgactctaagtggcattcaaggagtacctctctctagaactgtacgct
gtacctgcatcagcattagtaatcaacctgttaatccaaggtctttagaaa
aacttgaaattattcctgcaagccaattttgtccacgtgttgagatcattg
ctacaatgaaaaagaagggtgagaagagatgtctgaatccagaatcgaagg
ccatcaagaatttactgaaagcagttagcaaggaaaggtctaaaagatctc
ct

The amino acid sequence of CXCL 10 is provided below.

(SEQ ID NO: 514)
mnqtailicc lifltlsgiq gvplsrtvrc tcisisnqpv
nprsleklei ipasqfcprv eiiatmkkkg ekrclnpesk
aiknllkavs kerskrsp

His or myc tags are used for purification purposes and not needed for use as an antigen (e.g., for conjugation to a pHLIP® peptide).

### Small Molecule Antigens or Epitopes

Small molecule antigens are those that are characterized by a molecular mass of less than 2000 daltons. For example, the molecular mass of the small molecule antigen is preferably less than 1000 daltons, more preferably less than 600 daltons, e.g., the compound is less than 500 daltons, 400 daltons, 300 daltons, 200 daltons, or 100 daltons. Some small molecule antigens are characterized by their binding to endogenous antibodies in the blood or serum of many normal humans (or humans characterized as having a pathological tumor).

Exemplary small molecule epitopes are described below.

### Di-nitrophenvl (DNP)

Antibodies to DNP (0-(2,4-dinitrophenyl)hydroxylamine, shown below) have been identified in the IgG fraction of normal human sera.

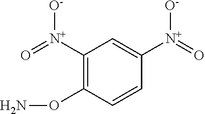 [SMILES: NOc1ccc([N+](=O)[O-])cc1[N+](=O)[O-]]

Thus, another small molecule antigen that binds endogenous antibodies is dinitrophenyl (DNP) and its derivatives. For example, N-(4-dimethylamino-3,5-dinitrophenyl)maleimide is shown below:

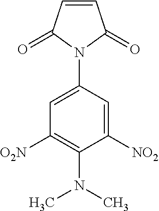 [SMILES: CN(C)c1c([N+](=O)[O-])cc(N2C(=O)C=CC2=O)cc1[N+](=O)[O-]]

OrDNP-PEG4-NHS (1-(2,4-Dinitrophenylamino)-3,6,9,12-tetraoxapentadecanoic acid succinimidyl ester) is shown below:

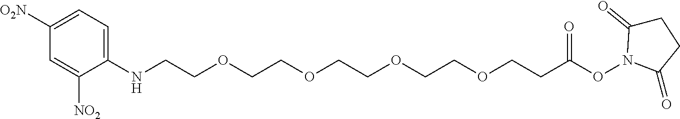 [SMILES: O=C(CCOCCOCCOCCOCCNc1ccc([N+](=O)[O-])cc1[N+](=O)[O-])ON1C(=O)CCC1=O]

DNP is a low molecular weight antigen known for its ability to bind antibodies in normal human serum. Binding of the antibodies to DNP delivered to the surface of tumor or otherwise diseased acidic cells leads to cytotoxicity of the antigen-labeled target cells. The DNP recruits endogenous antibodies, e.g., antibodies that exist in a subject prior to administration of the pHLIP® peptide constructs described herein.

Linker

In the schematic structure, Epitope-Linker-Peptide:

Linker could be relatively small, e.g., only a few atoms, to a rather large polar (or moderately hydrophobic) polymer or an N-terminal lengthening of the pHLIP® peptide by the addition of amino acids, e.g., glycine residues (poly-Gly). In some examples, a linker can be part of membrane non-inserting pHLIP® peptide sequence, such as those with a poly-Gly motif. In some examples, a linker could be PEG polymer. The purpose of a polymer or pHLIP® extension is to position epitopes at the surfaces of cells to enhance the access of antibodies or proteins for binding to the epitope. Non-limiting example of linker is a PEG polymer in size from 200 Da up to 20 kDa.

In some examples the following linkers and their derivatives could be used: N-α-maleimidoacet-oxysuccinimide ester (AMAS); N-γ-maleimidobutyryl-oxysuccinimide ester (GMBS); N-β-maleimidopropyl-oxysuccinimide ester (BMPS); N-ε-malemidocaproyl-oxysuccinimide ester (EMCS); m-maleimidobenzoyl-n-hydroxysuccinimide ester (MBS); succinimidyl 3-(bromoacetamido)propionate (SBAP); succinimidyl (4-iodoacetyl)aminobenzoate (SIAB); N-ε-maleimidocaproic acid (EMCA); succinimidyl 4-(n-maleimidomethyl)cyclohexane-1-carboxy-(6-amidocaproate) (LC-SMCC); succinimidyl iodoacetate (SIA); succinimidyl (4-iodoacetyl)aminobenzoate (SIAB); succinimidyl 4-(p-maleimidophenyl)butyrate (SMPB); succinimidyl 6-((beta-maleimidopropionamido)hexanoate) (SMPH); 3-propargyloxypropanoic acid, succinimidyl ester (alkyne, succinimidyl ester); 1,4-bismaleimidobutane (BMB); bismaleimidohexane (BMH); bismaleimidoethane (BMOE); tris(2-maleimidoethyl)amine (TMEA); N-β-maleimidopropionic acid hydrazide; (BMPH); N-ε-maleimidocaproic acid hydrazide (EMCH); N-κ-maleimidoundecanoic acid hydrazide (KMUH); 4-(4-n-maleimidophenyl)butyric acid hydrazide (MBPH); or p-maleimidophenyl isocyanate (PMPI).

In some examples the flexible linker or mucin domain is positioned between epitope and pHLIP® peptide.

The mucin-domain of CX3CL1 and the nucleic acid sequence encoding mucin-domain of CX3CL1 is described below:

SEQ ID NO: 500
aatggcggcaccttcgagaagcagatcggcgaggtgaagcccaggaccacc
cctgccgccgggggaatggacgagtctgtggtcctggagcccgaagccaca
ggcgaaagcagtagcctggagccgactccttcttcccaggaagcacagagg
gccctggggacctccccagagctgccgacgggtgtgactggttcctcaggg
accaggctccccccgacgccaaaggctcaggatggagggcctgtgggcacg
gagcttttccgagtgcctcccgtctccactgccgccacgtggcagagttct
gctccccaccaacctgggcccagcctctgggctgaggcaaagacctctgag
gccccgtccacccaggacccctccacccaggcctccactgcgtcctcccca
gccccagaggagaatgctccgtctgaaggccagcgtgtgtggggtcagggg
cagagccccaggccagagaactctctggagcgggaggagatgggtcccgtg
ccagcgcacacggatgccttccaggactgggggcctggcagcatggcccac
gtctctgtggtccctgtctcctcagaagggacccccagcagggagccagtg
gcttcaggcagctggacccctaaggctgaggaacccatccatgccaccatg
gacccccagaggctgggcgtccttatcactcctgtccctgacgcccaggct
gccacccggaggcag

The sequence of mucin-domain of the human CX3CL1 protein (UniProt P78423 or NP_002987.1) is shown below (these are residues 111-141 from the human CX3CL1 protein):

SEQ ID NO: 526
gtfekqigev kprttpaagg mdesvvlepe atgessslep
tpssqeaqra lgtspelptg vtgssgtrlp ptpkaqdggp
vgtelfrvpp vstaatwqss aphqpgpslw aeaktseaps
tqdpstqast asspapeena psegqrvwgq gqsprpensl
ereemgpvpa htdafqdwgp gsmahvsvvp vssegtpsre
pvasgswtpk aeepihatmd pqrlgvlitp vpdaqaatrr q

The full length CXCL1 amino acid sequence is depicted below (NP_002987.1), and incorporated herein by reference.

| SEQ ID NO: 527 | |
|---|---|
| 1 | mapislswll rlatfchltv llagqhhgvt kcnitcskmt skipvallih yqqnqascgk |
| 61 | raiiletrqh rlfcadpkeq wvkdamqhld rqaaaltrng gtfekqigev kprttpaagg |
| 121 | mdesvvlepe atgessslep tpssqeaqra lgtspelptg vtgssgtrlp ptpkaqdggp |
| 181 | vgtelfrvpp vstaatwqss aphqpgpslw aeaktseaps tqdpstqast asspapeena |
| 241 | psegqrvwgq gqsprpensl ereemgpvpa htdafqdwgp gsmahvsvvp vssegtpsre |
| 301 | pvasgswtpk aeepihatmd pqrlgvlitp vpdaqaatrr qavgllaflg llfclgvamf |
| 361 | tyqslqgcpr kmagemaegl ryiprscgsn syvlvpv |

Exemplary landmark residues, domains, and fragments of CXCL1 include, but are not limited to residues 1-24 (signal peptide), residues 25-397 (mature peptide), or residues 111-141 (as described above). A fragment (e.g., a peptide or an epitope) of a CXCL1 protein is less than the length of the full length protein, e.g., a fragment is at least 3, 4, 5, 6, 7, 8, 9, 10, 20, 30, 40, 50, 100, 200 or more residues in length, but less than e.g., 397 residues in the case of CXCL1 above.

pHLIP® Peptides

In the schematic structure, Epitope-Linker-Peptide:

Peptide is a pHLIP® peptide (non-limiting example is pHLIP® comprising the Var3 sequence AXDDQNPWRAYLDLLFPTDTLLLDLLWA (SEQ ID NO: 18) or AXDQDNPWRAYLDLLFPTDTLLLDLLWA (SEQ ID NO: 495), where X is a functional group for conjugation purposes, selected from a lysine (Lys), a cysteine (Cys), or Azido-containing amino acid or others. The membrane non-inserting N-terminal flanking sequence of pHLIP® peptide can be extend. For example, the pHLIP® peptide shares the sequence: WRAYLDLLFPTDTLLLDLLW (SEQ ID NO: 112).

Non-limiting examples of the extension can be poly-Gly motif or AX(Z)nXPWRAYLDLLFPTDTLLLDLLWA (SEQ ID NO: 496), where “Z” indicates any amino acid residue, and n is any integer between and including 1-10 (e.g., 1≤n≤10). Additionally, (Z)n could be QDNDQN (SEQ ID NO: 497) or NENENN (SEQ ID NO: 528) or NDNDNN (SEQ ID NO: 529) or NDNDNDN (SEQ ID NO:530), any combination of polar residues, D, E, N or Q.

An example of a wild type (WT) pHLIP® peptide is AXEQNPIYWARYADWLFTTPLLLLDLALLVDADEGT (SEQ ID NO: 20) where X is a functional group for conjugation purposes, selected from a lysine (Lys), a cysteine (Cys), an Azido-containing amino acid, or others, and in which AEQNPIY (SEQ ID NO: 501) represents a flanking sequence, WARYADWLFTTPLLLLDLALLV (SEQ ID NO: 21) represents a membrane-inserting sequence, and DADEGT represents a flanking sequence.

The constructs may include a pHLIP® peptide with a N-terminal extension. For example, the N terminus of any of these peptide sequences can be extended by the addition of amino acids to space the epitope away from the cell surface, e.g. by including a (glycine) extension.

Other exemplary pHLIP® peptides are shown in the Tables below.

TABLE 1
Exemplary pHLIP ® peptides

| Name | Sequence | SEQ ID No. |
|---|---|---|
| Var3-1a | ACDQDNPWRAYLDLLFPTDTLLLDLLWA | SEQ. ID NO. 537 |
| Var3-1b | AKDQDNPWRAYLDLLFPTDTLLLDLLWA | SEQ. ID NO. 538 |
| Var3-2a | ACQDNDQNCPWRAYLDLLFPTDTLLLDLLWA | SEQ. ID NO. 539 |
| Var3-2b | AKQDNDQNKPWRAYLDLLFPTDTLLLDLLWA | SEQ. ID NO. 540 |
| WT-1 | GGEQNPIYWARYADWLFTTPLLLLDLALLVDADEGT | SEQ. ID NO. 22 |
| WT-2 | AEQNPIYWARYADWLFTTPLLLLDLALLVDADEGT | SEQ. ID NO. 23 |
| Var3-WT-Cys | ADDQNPWRAYLDLLFPTDTLLLDLLWDADECG | SEQ. I NO. 24 |
| Cys-Var3-WT | ACDDQNPWRAYLDLLFPTDTLLLDLLWDADEG | SEQ. I NO. 25 |
| Lys-Var3-WT | AKDDQNPWRAYLDLLEPTDTELLDLLWDADEG | SEQ. I NO. 26 |
| WT-Cys1 | AAEQNPIYWARYADWLFTTPLLLLDLALLVDADEGTCG | SEQ. ID NO. 27 |
| WT-Cys2 | Ac-AEQNPIYWARYADWLFTTPLLLLDLALLVDADEGCT | SEQ ID NO: 28 |
| WT-Cys3 | GGEQNPIYWARYADWLFTTPLLLLDLALLVDADEGTCG | SEQ. ID NO. 29 |
| Cys-WT1 | Ac-ACEQNPIYWARYADWLFTTPLLLLDLALLVDADEGTG | SEQ. ID NO. 30 |
| Var0-NT | ACEQNPIYWARYADWLFTTPLLLLDLALLVDADEGT | SEQ. ID NO. 31 |
| Lys-WT1 | AKEQNPIYWARYADWLFTTPLLLLDLALLVDADEGT | SEQ. ID NO. 32 |
| Lys-WT2 | Ac-AKEQNPIYWARYADWLFTTPLLLLDLALLVDADEGTG | SEQ ID NO: 33 |
| WT-KC | AAEQNPIYWARYADWLFTTPLLLLDLALLVDADEGTKCG | SEQ. ID NO. 34 |
| K-WT-C | AKEQNPIYWARYADWLFTTPLLLLDLALLVDADECT | SEQ. ID NO. 35 |
| N-pHLIP ® | ACEQNPIYWARYANWLFTTPLLLLNLALLVDADEGTG | SEQ. ID NO. 36 |
| N-pHLIP ®-b | ACEQNPIYWARYANWLFTTPLLLLNLALLVDADEGT | SEQ ID NO: 37 |
| K-pHLIP ® | ACEQNPIYWARYAKWLFTTPLLLLKLALLVDADEGTG | SEQ. ID NO. 38 |
| NNQ | GGEQNPIYWARYADWLFTTPLLLLDLALLVNANQGT | SEQ. ID NO. 39 |
| D25A | AAEQNPIYWARYADWLFTTPLLLLALALLVDADEGT | SEQ. ID NO. 40 |
| D25A-KC | Ac-AAEQNPIYWARYADWLFTTPLLLLELALLVDADEGTKCG | SEQ ID NO: 41 |
| D14A | AAEQNPIYWARYAAWLFTTPLLLLDLALLVDADEGT | SEQ. ID NO. 42 |
| P20A | AAEQNPIYWARYADWLFTTALLLLDLALLVDADEGT | SEQ. ID NO. 43 |
| D25E | AAEQNPIYWARYADWLFTTPLLLLELALLVDADEGT | SEQ. ID NO. 44 |
| D14E | AAEQNPIYWARYAEWLFTTPLLLLDLALLVDADEGT | SEQ. ID NO. 45 |
| 3D | AAEQNPIIYWARYADWLFTDLPLLLLDLLALLVDADEGT | SEQ. ID NO. 46 |
| R11Q | GEQNPIYWAQYADWLFTTPLLLLDLALLVDADEGTCG | SEQ. ID NO. 47 |
| D25Up | GGEQNPIYWARYADWLFTTPLLLDLLALLVDADEGTCG | SEQ. ID NO. 48 |
| D25Down | GGEQNPIYWARYADWLFTTPLLLLLDALLVDADEGTCG | SEQ. ID NO. 49 |
| D14Up | GGEQNPIYWARYDAWLFTTPLLLLDLALLVDADEGTCG | SEQ. ID NO. 50 |
| D14Down | GGEQNPIYWARYAWDLFTTPLLLLDLALLVDADEGTCG | SEQ. ID NO. 51 |
| P20G | AAEQNPIYWARYADWLFTTGLLLLDLALLVDADEGT | SEQ. ID NO. 52 |
| H1-Cys | DDDEDNPIYWARYADWLFTTPLLLLHGALLVDADECT | SEQ. ID NO. 53 |
| H1 | DDDEDNPIYWARYADWLFTTPLLLLHGALLVDADET | SEQ ID NO: 54 |
| H2-Cys | DDDEDNPIYWARYAHWLFTTPLLLLHGALLVDADEGCT | SEQ. ID NO. 55 |
| Cys-H2 | CDDDEDNPIYWARYAHWLFTTPLLLLHGALLVDADET | SEQ ID NO: 56 |
| H2 | DDDEDNPIYWARYAHWLFTTPLLLLHGALLVDADEGT | SEQ ID NO: 57 |
| H2N-Cys | DDDEDNPIYWARYAHWLFTTPLLLLHGALLVNADECT | SEQ. ID NO. 58 |
| H2N | DDDEDNPIYWARYAHWLFTTPLLLLHGALLVNADEGT | SEQ ID NO: 59 |
| H2N2-Cys | DDDEDNPIYWARYAHWLFTTPLLLLHGALLVNANECT | SEQ. ID NO. 60 |
| H2N2 | DDDEDNPIYWARYAHWLFTTPLLLLHGALLVNANEGT | SEQ ID NO: 61 |
| 1a-Trp | AEQNPIYWARYADFLFTTPLLLLDLALLVDADET | SEQ. ID NO. 62 |
| 1b-Trp | AEQNPIYFARYADWLFTTPLLLLDLALLVDADEGT | SEQ. ID NO. 63 |
| 1c-Trp | AEQNPIYFARYADFLFTTPLLLLDLALLWDADET | SEQ. ID NO. 64 |
| Fast-1 or Var1 | AKEDQNPYWARYADWLFTTPLLLLDLALLVDG | SEQ. ID NO. 65 |
| Var1-2D1D | ACEDQNPYWARYADWLFTTPLLLLDLALLVDG | SEQ. ID NO. 66 |
| Fast1-Cys or | AEDQNPYWARYADWLFTTPLLLLDLALLVDCG | SEQ. ID NO. 67 |
| Var1-2D1D-Cys | | |
| Fast1-E-Cys or | AEDQNPYWARYADWLFTTPLLLLELALLVECG | SEQ. ID NO. 68 |
| Var1E | | |
| Fast1-E-Lys | AKEDQNDPYWARYADWLFTTPLLLLDLALLVG | SEQ ID NO: 69 |
| Fast2 or Var2 | AKEDQNPYWRAYADLFTPLTLLDLLALWDG | SEQ. ID NO. 70 |
| Fast2-E-Cys or | AEDQNPYWARYADWLFTTPLLLLELALLVCG | SEQ ID NO: 71 |
| Var2E | | |
| Var2-2D1D | ACEDQNPYWRAYADLFTPLTLLDLLALWDG | SEQ. ID NO. 72 |
| Var3-3D | ACDDQNPWRAYLDLLFPTDTLLLDLLW | SEQ. ID NO. 73 |
| Var3-3D-cys | AKDDQNPWRAYLDLLFPTDTLLLDLLWC | SEQ ID NO: 74 |
| Var4-3E | ACEEQNPWRAYLELLFPTETLLLELLW | SEQ ID NO: 75 |
| Var5-3Da | ACDDQNPWARYLDWLFPTDTLLLDL | SEQ ID NO: 76 |
| Var6-3Db | CDNNNPWRAYLDLLFPTDTLLLDW | SEQ ID NO: 77 |
| Var8-3Eb | CEEQQPWAQYLELLFPTETLLLEW | SEQ ID NO: 78 |
| Var9-3Ec | CEEQQPWRAYLELLFPTETLLLEW | SEQ ID NO: 79 |
| Var15-2N | CDDDDDNPNYWARYANWLFTTPLLLLNGALLVEAEET | SEQ ID NO: 80 |
| Var16-2P | CDDDDDNPNYWARYAPWLFTTPLLLLPGALLVEAEE | SEQ ID NO: 81 |

TABLE 2
Exemplary pHLIP ® peptides

| Name | Sequence | SEQ ID No. |
|---|---|---|
| Var14- | Ac-TEDADVLLALDLLLLPTTFLW | SEQ. ID NO. 82 |
| Rev | DAYRAWYPNQECA-Am | |
| Sh | AEQNPIYWARYADWLFTTPL | SEQ. ID NO. 83 |
| Sh-Cys | AEQNPIYWARYADWLFTTPCL | SEQ. ID NO. 84 |
| Cys-Sh | ACEQNPIYWARYADWLFTTPL | SEQ. ID NO. 85 |
| Sh-1Trp | AEQNPIYFARYADWLFTTPL | SEQ. ID NO. 86 |
| Sh-W2 | AEQNPIYFARYADLLFPTTLAW | SEQ ID NO: 87 |
| Sh-W1 | AEQNPIYWARYADLLFPTTLAF | SEQ ID NO: 88 |
| Sh-2W | AEQNPIYWARYADLLFPTTLAW | SEQ ID NO: 89 |
| Sh-1D | KEDQNPWARYADLLFPTTLAW | SEQ. ID NO. 90 |
| Sh-1Db | KEDQNPWARYADLLFPTTLW | SEQ ID NO: 91 |
| Var12-1D | ACEDQNPWARYADLLFPTTLAW | SEQ. ID NO. 92 |
| Var10-2D | ACEDQNPWARYADWLFPTTLLLLD | SEQ. ID NO. 93 |
| Var13-1E | ACEEQNPWARYAELLFPTTLAW | SEQ. ID NO. 94 |
| Var11-2E | ACEEQNPWARYAEWLFPTTLLLLE | SEQ. ID NO. 95 |
| Var7-3E | ACEEQNPWARYLEWLFPTETLLLEL | SEQ. ID NO. 96 |
| Var7-3Eb | ACEEQNPQAEYAEWLFPTTLLLLE | SEQ ID NO: 97 |

“Ac” means Acetylated N-terminus
“Am” means Amidated C-terminus

TABLE 3
Coded and exemplary non-coded amino acids including
L-isomers, D-isomers, alpha-isomers, beta-isomers,
glycol-, and methyl- modifications.

| No. | Abbrev | Name |
|---|---|---|
| 1 | Ala | Alanine |
| 2 | Arg | Arginine |
| 3 | Asn | Asparagine |
| 4 | Asp | Aspartic acid |
| 5 | Cys | Cysteine |
| 6 | Gln | Glutamine |
| 7 | Glu | Glutamic acid |
| 8 | Gly | Glycine |
| 9 | His | Histidine |
| 10 | Ile | Isoleucine |
| 11 | Leu | Leucine |
| 12 | Lys | Lysine |
| 13 | Met | Methionine |
| 14 | Phe | Phenylalanine |
| 15 | Pro | Proline |
| 16 | Ser | Serine |
| 17 | Thr | Threonine |
| 18 | Trp | Tryptophan |
| 19 | Tyr | Tyrosine |
| 20 | Val | Valine |
| 21 | Sec | Selenocysteine |
| 22 | Sem | Selenomethionine |
| 23 | Pyl | Pyrrolysine |
| 24 | Aad | Alpha-aminoadipic acid |
| 25 | Acpa | Amino-caprylic acid |
| 26 | Aecys | Aminoethyl cysteine |
| 27 | Afa | Aminophenyl acetate |
| 28 | Gaba | Gamma-aminobutyric acid |
| 29 | Aiba | Aminoisobutyric acid |
| 30 | Aile | Alloisoleucine |
| 31 | AIg | Allylglycine |
| 32 | Aba | Amino-butyric acid |
| 33 | Aphe | Amino-phenylalanine |
| 34 | Brphe | Bromo-phenylalanine |
| 35 | Cha | Cyclo-hexylalanine |
| 36 | Cit | Citrulline |
| 37 | Clala | Chloroalanine |
| 38 | Cie | Cycloleucine |
| 39 | Clphe | Fenclonine (or chlorophenylalanine) |
| 40 | Cya | Cysteic acid |
| 41 | Dab | Diaminobutyric acid |
| 42 | Dap | Diaminopropionic acid |
| 43 | Dap | Diaminopimelic acid |
| 44 | Dhp | Dehydro-proline |
| 45 | Dhphe | DOPA (or 3,4-dihydroxyphenylalanine) |
| 46 | Fphe | Fluorophenylalanine |
| 47 | Gaa | Glucosaminic acid |
| 48 | Gla | Gamma-carboxyglutamic acid |
| 49 | Hag | Homoarginine |
| 50 | Hlys | Hydroxylysine |
| 51 | Hnvl | Hydroxynorvaline |
| 52 | Hog | Homoglutamine |
| 53 | Hoph | Homophenylalanine |
| 54 | Has | Homoserine |
| 55 | Hse | Homocysteine |
| 56 | Hpr | Hydroxyproline |
| 57 | Iphe | Iodo-phenylalanine |
| 58 | Ise | Isoserine |
| 59 | Mle | Methyl-leucine |
| 60 | Msmet | Methionine-methylsulfonium chloride |
| 61 | Nala | Naphthyl-alanine |
| 62 | Nle | Norleucine (or 2-aminohexanoic acid) |
| 63 | Nmala | N-methyl-alanine |
| 64 | Nva | Norvaline (or 2-aminopentanoic acid) |
| 65 | Obser | O-benzyl-serine |
| 66 | Obtyr | O-benzyl-tyrosine |
| 67 | Oetyr | O-ethyl-tyrosine |
| 68 | Omser | O-methyl-serine |
| 69 | Omthr | O-methy-threonine |
| 70 | Omtyr | O-methyl-tyrosine |
| 71 | Orn | Ornithine |
| 72 | Pen | Penicillamine |
| 73 | Pga | Pyroglutamic acid |
| 74 | Pip | Pipecolic acid |
| 75 | Sar | Sarcosine |
| 76 | Tfa | Trifluoro-alanine |
| 77 | Thphe | Hydroxy-Dopa |
| 78 | Vig | Vinylglycine |
| 79 | Aaspa | Amino-aminoethylsulfanylpropanoic acid |
| 80 | Ahdna | Amino-hydroxy-dioxanonanolic acid |
| 81 | Ahoha | Amino-hydroxy-oxahexanoic acid |
| 82 | Ahsopa | Amino-hydroxyethylsulfanylpropanoic acid |
| 83 | Tyr(Me) | Methoxyphenyl-methylpropanyl oxycarbonylamino |
| | | propanoic acid |
| 84 | MTrp | Methyl-tryptophan |
| 85 | pTyr | Phosphorylated Tyr |
| 86 | pSer | Phosphorylated Ser |
| 87 | pThr | Phosphorylated Thr |
| 88 | BLys | BiotinLys |
| 89 | Hyp | Hydroproline |
| 90 | Phg | Phenylglycine |
| 91 | Cha | Cyclohexyl-alanine |
| 92 | Chg | Cyclohexylglycine |
| 93 | Nal | Naphthylalanine |
| 94 | Pal | Pyridyl-alanine |
| 95 | Pra | Propargylglycine |
| 96 | Gly(allyl) | Pentenoic acid |
| 97 | Pen | Penicillamine |
| 98 | MetO | Methionine sulfoxide |
| 99 | Pca | Pyroglutamic acid |
| 100 | Ac-Lys | Acetylation of Lys |

TABLE 4
Non-limiting examples of protonatable residues
and their substitutions including L-isomers,
D-isomers, alpha-isomers, and beta-isomers.

| Original Residue | Exemplary amino acids substitution |
|---|---|
| Asp (D) | Glu (E); Gla (Gla); Aad (Aad) |
| Glu (E) | Asp (D); Gla (Gla); Aad (Aad) |

TABLE 5
Examples of coded amino acid substitutions

| Original Residue | Substitution |
|---|---|
| Ala (A) | Gly; Ile; Leu; Met; Phe; Pro; Trp; Tyr; Val |
| Arg (R) | Lys |
| Asn (N) | Gln; His |
| Asp (D) | Glu |
| Cys (C) | Ser; Met |
| Gln (Q) | Asn; His |
| Glu (E) | Asp |
| Gly (G) | Ala; Ile; Leu; Met; Phe; Pro; Trp; Tyr; Val |
| His (H) | Asn; Gln |
| Ile (I) | Ala; Gly; Leu; Met; Phe; Pro; Trp; Tyr; Val |
| Leu (L) | Ala; Gly; Ile; Met; Phe; Pro; Trp; Tyr; Val |
| Lys (K) | Arg |
| Met (M) | Ala; Gly; Leu; Ile; Phe; Pro; Trp; Tyr; Val |
| Phe (F) | Ala; Gly; Leu; Ile; Met; Pro; Trp; Tyr; Val |
| Pro (P) | Ala; Gly; Leu; Ile; Met; Phe; Trp; Tyr; Val |
| Ser (S) | Thr |
| Thr (T) | Ser |
| Trp (W) | Ala; Gly; Leu; Ile; Met; Pro; Phe; Tyr; Val |
| Tyr (Y) | Ala; Gly; Leu; Ile; Met; Pro; Phe; Trp; Val |
| Val (V) | Ala; Gly; Leu; Ile; Met; Pro; Phe; Trp; Tyr |

```
TABLE 6
Non-limiting examples of membrane-inserting
sequences belonging to different groups of
pHLIP ® peptides. Each protonatable residue
(shown in bold) could be replaced by its
  substitution from Table 4. Each non-polar
  residue could be replaced by its coded amino
  acid substitution from Table 5, and/or non-
coded amino acid substitutions from Table 3.
```

| Groups | Sequences |
|---|---|
| WT-BRC | WARYADWLFTTPLLLLDLALL (SEQ ID NO: 98) |
| | YARYADWLFTTPLLLLDLALL (SEQ ID NO: 99) |
| | WARYSDWLFTTPLLLYDLGLL (SEQ ID NO: 100) |
| | WARYTDWFTTPLLLYDLALLA (SEQ ID NO: 101) |
| | WARYTDWLFTTPLLLYDLGLL (SEQ ID NO: 102) |
| | WARYADWLFTTPLLLLDLSLL (SEQ ID NO: 103) |
| WT-BRC | LLALDLLLLPTTFLWDAYRAW (SEQ ID NO: 104) |
| Reverse | LLALDLLLLPTTFLWDAYRAY (SEQ ID NO: 105) |
| | LLGLDYLLLPTTFLWDSYRAW (SEQ ID NO: 106) |
| | ALLALDYLLLPTTFWDTYRAW (SEQ ID NO: 107) |
| | LLGLDYLLLPTTFLWDTYRAW (SEQ ID NO: 108) |
| | LLSLDLLLLPTTFLWDAYRAW (SEQ ID NO: 109) |
| ATRAM | GLAGLLGLEGLLGLPLGLLEGLWLGL (SEQ ID |
| | NO: 110) |
| ATRAM | LGLWLGELLGLPLGLLGELGLLGALG (SEQ ID |
| Reverse | NO: 111) |
| Var3 | WRAYLDLLFPTDTLLLDLLW (SEQ ID NO: 112) |
| Var3 | WLLDLLLTDTPFLLDLYARW (SEQ ID NO: 113) |
| Reverse | |
| Var7 | WARYLEWLFPTETLLLEL (SEQ ID NO: 114) |
| | WAQYLELLFPTETLLLEW (SEQ ID NO: 115) |
| Var7 | LELLLTETPFLWELYRAW (SEQ ID NO: 116) |
| Reverse | WELLLTETPFLLELYQAW (SEQ ID NO: 117) |
| Single | WLFTTPLLLLNGALLVE (SEQ ID NO: 118) |
| D/E | WLFTTPLLLLPGALLVE (SEQ ID NO: 119) |
| | WARYADLLFPTTLAW (SEQ ID NO: 120) |
| Single | EVLLAGNLLLLPTTFLW (SEQ ID NO: 121) |
| D/E | EVLLAGPLLLLPTTFLW (SEQ ID NO: 122) |
| Reverse | WALTTPFLLDAYRAW (SEQ ID NO: 123) |
| pHLIP ®- | NLEGFFATLGGEIALWSLVVLAIE (SEQ ID NO: 124) |
| Rho | EGFFATLGGEIALWSDVVLAIE (SEQ ID NO: 125) |
| | EGFFATLGGEIPLWSDVVLAIE (SEQ ID NO: 126) |
| pHLIP ®- | EIALVVLSWLAIEGGLTAFFGELN (SEQ ID NO: 127) |
| Rho | EIALVVDSWLAIEGGLTAFFGE (SEQ ID NO: 128) |
| Reverse | EIALVVDSWLPIEGGLTAFFGE (SEQ ID NO: 129) |
| pHLIP ®- | ILDLVFGLLFAVTSVDFLVQW (SEQ ID NO: 130) |
| CA9 | |
| pHLIP ®- | WQVLFDVSTVAFLLGFVLDLI (SEQ ID NO: 131) |
| CA9 | |
| Reverse | |

TABLE 7
Non-limiting examples of pHLIP ® sequences. A cysteine, a lysine,
an azido-modified amino acid, or an alkynyl modified amino acid
can be incorporated at the N-terminal (first 6 residues) or C-
terminal (last 6 residues) parts of the peptides for conjugation
with a cargo, and a linker.

| SEQ ID NO | Name | Sequence |
|---|---|---|
| SEQ ID NO: 132 | WT-2D | AEQNPIYWARYADWLFTTPLLLLDLALLVDADET |
| SEQ ID NO: 133 | WT-6E | AEQNPIYWARYAEWLFTTPLLLLELALLVEAEET |
| SEQ ID NO: 134 | WT-3D | ADDQNPWRAYLDLLFPDTTDLLLLDLLWDADET |
| SEQ ID NO: 135 | WT-9E | AEEQNPWRAYLELLFPETTELLLLELLWEAEET |
| SEQ ID NO: 136 | WT-GlaD | AEQNPIYWARYAGlaWLFTTPLLLLDLALLVDADET |
| SEQ ID NO: 137 | WT-DGla | AEQNPIYWARYADWLFTTPLLLLGlaLALLVDADET |
| SEQ ID NO: 138 | WT-2G1a | AEQNPIYWARYAGlaWLFTTPLLLLGlaLALLVDADET |
| SEQ ID NO: 139 | WT-AadD | AEQNPIYWARYAAadWLFTTPLLLLDLALLVDADET |
| SEQ ID NO: 140 | WT-DAad | AEQNPIYWARYADWLFTTPLLLLAadLALLVDADET |
| SEQ ID NO: 141 | WT-2Aad | AEQNPIYWARYAAadWLFTTPLLLLAadLALLVDADET |
| SEQ ID NO: 142 | WT-GlaAad | AEQNPIYWARYAGlaWLFTTPLLLLAadLALLVDADET |
| SEQ ID NO: 143 | WT-AadGla | AEQNPIYWARYAAadWLFTTPLLLLGlaLALLVDADET |
| SEQ ID NO: 144 | WT-1 | GGEQNPIYWARYADWLFTTPLLLLDLALLVDADEGT |
| SEQ ID NO: 145 | WT-2 | GGEQNPIYWARYADWLFTTPLLLLDLALLVDADEGT |
| SEQ ID NO: 146 | WT-3 | AAEQNPIYWARYADWLFTTPLLLLDLALLVDADEGT |
| SEQ ID NO: 147 | WT-4 | AEQNPIYWARYADWLFTTPLLLLDLALLVDADEGT |
| SEQ ID NO: 148 | WT-2N | AEQNPIYWARYANWLFTTPLLLLNLALLVDADEGT |
| SEQ ID NO: 149 | WT-2K | AEQNPIYWARYAKWLFTTPLLLLKLALLVDADEGT |
| SEQ ID NO: 150 | WT-2DNANQ | GGEQNPIYWARYADWLFTTPLLLLDLALLVNANQGT |
| SEQ ID NO: 151 | WT-D25A | AAEQNPIYWARYADWLFTTPLLLLALALLVDADEGT |
| SEQ ID NO: 152 | WT-D14A | AAEQNPIYWARYAAWLFTTPLLLLDLALLVDADEGT |
| SEQ ID NO: 153 | WT-P20A | AAEQNPIYWARYADWLFTTALLLLDLALLVDADEGT |
| SEQ ID NO: 154 | WT-D25E | AAEQNPIYWARYADWLFTTPLLLLELALLVDADEGT |
| SEQ ID NO: 155 | WT-D14E | AAEQNPIYWARYAEWLFTTPLLLLDLALLVDADEGT |
| SEQ ID NO: 156 | WT-3D-2 | AAEQNPIIYWARYADWLFTDLPLLLLDLLALLVDADEGT |
| SEQ ID NO: 157 | WT-R11Q | GEQNPIYWAQYADWLFTTPLLLLDLALLVDADEG |
| SEQ ID NO: 158 | WT-D25Up | GGEQNPIYWARYADWLFTTPLLLDLLALLVDADEG |
| SEQ ID NO: 159 | WT-D25Down | GGEQNPIYWARYADWLFTTPLLLLLDALLVDADEG |
| SEQ ID NO: 160 | WT-D14Up | GGEQNPIYWARYDAWLFTTPLLLLDLALLVDADEGT |
| SEQ ID NO: 161 | WT-D14Down | GGEQNPIYWARYAWDLFTTPLLLLDLALLVDADEG |
| SEQ ID NO: 162 | WT-P20G | AAEQNPIYWARYADWLFTTGLLLLDLALLVDADEGT |
| SEQ ID NO: 163 | WT-DH | DDDEDNPIYWARYADWLFTTPLLLLHGALLVDAD |
| SEQ ID NO: 164 | WT-2H | DDDEDNPIYWARYAHWLFTTPLLLLHGALLVDADE |
| SEQ ID NO: 165 | WT-L16H | CEQNPIYWARYADWHFTTPLLLLDLALLVDADE |
| SEQ ID NO: 166 | WT-1Wa | AEQNPIYWARYADFLFTTPLLLLDLALLVDADET |
| SEQ ID NO: 167 | WT-1Wb | AEQNPIYFARYADWLFTTPLLLLDLALLVDADE |
| SEQ ID NO: 168 | WT-1Wc | AEQNPIYFARYADFLFTTPLLLLDLALLWDADET |
| SEQ ID NO: 169 | WT-W6 | ADNNPWIYARYADLTTFPLLLLDLALLVDFDD |
| SEQ ID NO: 170 | WT-W17 | ADNNPFIYARYADLTTWPLLLLDLALLVDFDD |
| SEQ ID NO: 171 | WT-W30 | ADNNPFIYARYADLTTFPLLLLDLALLVDWDD |
| SEQ ID NO: 172 | WT-W17-P7 | ADNNPFPYARYADLTTWILLLLDLALLVDFDD |
| SEQ ID NO: 173 | WT-W39-R11 | ADNNPFIYAYRADLTTFPLLLLDLALLVDWDD |
| SEQ ID NO: 174 | WT-W30-R15 | ADNNPFIYATYADLRTFPLLLLDLALLVDWDD |
| SEQ ID NO: 175 | WT-Rev | Ac-TEDADVLLALDLLLLPTTFLWDAYRAWYPNQEA-Am |
| SEQ ID NO: 176 | Var1-3D | AEDQNPYWARYADWLFTTPLLLLDLALLVD |
| SEQ ID NO: 177 | Var1-1D2E | AEDQNPYWARYADWLFTTPLLLLELALLVE |
| SEQ ID NO: 178 | Var2-3D | AEDQNPYWRAYADLFTPLTLLDLLALWD |
| SEQ ID NO: 179 | Var3-3D | ADDQNPWRAYLDLLFPTDTLLLDLLW |
| SEQ ID NO: 180 | Var3-WT | ADDQNPWRAYLDLLFPTDTLLLDLLWDADE |
| SEQ ID NO: 181 | Var3-Gla2D | ADDQNPWRAYLGlaLLFPTDTLLLDLLW |
| SEQ ID NO: 182 | Var3-DGlaD | ADDQNPWRAYLDLLFPTGlaTLLLDLLW |
| SEQ ID NO: 183 | Var3-2DGla | ADDQNPWRAYLDLLFPTDTLLLGlaLLW |
| SEQ ID NO: 184 | Var3-2GlaD | ADDQNPWRAYLGlaLLFPTGlaTLLLDLLW |
| SEQ ID NO: 185 | Var3-GlaDGla | ADDQNPWRAYLGlaLLFPTDTLLLGlaLLW |
| SEQ ID NO: 186 | Var3-D2Gla | ADDQNPWRAYLDLLFPTGlaTLLLGlaLLW |
| SEQ ID NO: 187 | Var3-3Gla | ADDQNPWRAYLGlaLLFPTGlaTLLLGlaLLW |
| SEQ ID NO: 188 | Var3-Aad2D | ADDQNPWRAYLAadLLFPTDTLLLDLLW |
| SEQ ID NO: 189 | Var3-DAadD | ADDQNPWRAYLDLLFPTAadTLLLDLLW |
| SEQ ID NO: 190 | Var3-2DAad | ADDQNPWRAYLDLLFPTDTLLLAadLLW |
| SEQ ID NO: 191 | Var3-2AadD | ADDQNPWRAYLAadLLFPTAadTLLLDLLW |
| SEQ ID NO: 191 | Var3-AadDAad | ADDQNPWRAYLAadLLFPTDTLLLAadLLW |
| SEQ ID NO: 192 | Var3-D2Aad | ADDQNPWRAYLDLLFPTAadTLLLAadLLW |
| SEQ ID NO: 193 | Var3-3Aad | ADDQNPWRAYLAadLLFPTAadTLLLAadLLW |
| SEQ ID NO: 194 | Var3-GlaAadD | ADDQNPWRAYLGlaLLFPTAadTLLLDLLW |
| SEQ ID NO: 195 | Var3-GlaDAad | ADDQNPWRAYLGlaLLFPTDTLLLAadLLW |
| SEQ ID NO: 196 | Var3-2GlaAad | ADDQNPWRAYLGlaLLFPTGlaTLLLAadLLW |
| SEQ ID NO: 197 | Var3-AadGlaD | ADDQNPWRAYLAadLLFPTGlaTLLLDLLW |
| SEQ ID NO: 198 | Var3-AadDGla | ADDQNPWRAYLAadLLFPTDTLLLGlaLLW |
| SEQ ID NO: 199 | Var3-GlaAadGla | ADDQNPWRAYLGlaLLFPTAadTLLLGlaLLW |
| SEQ ID NO: 200 | Var3-GLL | GEEQNPWLGAYLDLLFPLELLGLLELGLW |
| SEQ ID NO: 201 | Var3-M | ADDDDDDPWQAYLDLLFPTDTLLLDLLW |
| SEQ ID NO: 202 | Var4-3E | AEEQNPWRAYLELLFPTETLLLELLW |
| SEQ ID NO: 203 | Var5-3Da | ADDQNPWARYLDWLFPTDTLLLDL |
| SEQ ID NO: 204 | Var6-3Db | DNNNPWRAYLDLLFPTDTLLLDW |
| SEQ ID NO: 205 | Var7-3E | AEEQNPWARYLEWLFPTETLLLEL |
| SEQ ID NO: 206 | Var7-M | DDDDDDPWQAYLDLFPTDTLALDLW |
| SEQ ID NO: 207 | Var8-3E | EEQQPWAQYLELLFPTETLLLEW |
| SEQ ID NO: 208 | Var9-3E | EEQQPWRAYLELLFPTETLLLEW |
| SEQ ID NO: 209 | Var10-2D | AEDQNPWARYADWLFPTTLLLLD |
| SEQ ID NO: 210 | Var11-2E | AEEQNPWARYAEWLFPTTLLLLE |
| SEQ ID NO: 211 | Var12-1D | AEDQNPWARYADLLFPTTLAW |
| SEQ ID NO: 212 | Var13-1E | AEEQNPWARYAELLFPTTLAW |
| SEQ ID NO: 213 | Var15-2N | DDDDDNPNYWARYANWLFTTPLLLLNGALLVEAEET |
| SEQ ID NO: 214 | Var16-2P | DDDDDNPNYWARYAPWLFTTPLLLLPGALLVEAEET |
| SEQ ID NO: 215 | Var17 | AEQNPIYFARYADFLFTTPLLLLDLALLWDADET |
| SEQ ID NO: 216 | Var18 | AEQNPIYWARYADFLFTTPLLLLDLALLVDADET |
| SEQ ID NO: 217 | Var19a | AEQNPIYWARYADWLFTTPL |
| SEQ ID NO: 218 | Var20 | AEQNPIYFARYADLLFPTTLAW |
| SEQ ID NO: 219 | Var21 | AEQNPIYWARYADLLFPTTLAF |
| SEQ ID NO: 220 | Var22 | AEQNPIYWARYADLLFPTTLAW |
| SEQ ID NO: 221 | Var23 | AEQNPIYFARYADWLFTTPL |
| SEQ ID NO: 222 | Var24 | EDQNPWARYADLLFPTTLAW |
| SEQ ID NO: 223 | ATRAM | GLAGLAGLLGLEGLLGLPLGLLEGLWLGLELEGN |
| SEQ ID NO: 224 | pHLIP ®-CA9 | EQNPIYILDLVFGLLFAVTSVDFLVQWDDAGD |
| SEQ ID NO: 225 | pHLIP ®-Rho | NLEGFFATLGGEIALWSLVVLAIE |
| SEQ ID NO: 226 | pHLIP ®-RhoM1 | NNEGFFATLGGEIALWSDVVLAIE |
| SEQ ID NO: 227 | pHLIP ®-RhoM2 | DNNEGFFATLGGEIPLWSDVVLAIE |

Epitopes may also be delivered to the cell surface of target cells (tumor cells and other diseased tissues/cells) using cyclic pHLIP® peptides. A cyclic peptide is one that comprises a circle geometry or structure. For example, the entire structure of the peptide is circular or a portion of the structure is circular. For example, in the latter case the peptide comprises a cyclic portion and a linear (or tail) portion. In various embodiments, a pH triggered peptide comprises at least 4 amino acids, where (a) at least 2 of the at least 4 amino acids of the peptide are non-polar amino acids, (b) at least 1 of the at least 4 amino acids of the peptide is a protonatable amino acid, and (c) the peptide has a higher affinity to a membrane lipid bilayer at pH 5.0 compared to at pH 8.0. Such pHLIP® peptides are described in International Patent Application No. PCT/US2017/023458 (PCT publication no. WO2017/165452A1, hereby incorporated by reference.

Exemplary cyclic pHLIP® peptides are described and shown below. A lowercase “c” at the beginning of a sequence herein denotes a cyclic peptide (e.g., as in c[(WE)3WC]) (SEQ ID NO: 1), and a lowercase “1” denotes a linear peptide (e.g., as in 1(CW(EW)4)) (SEQ ID NO: 188). In the case of cyclic structures that comprise a tail, the cyclic portion of the compound is within brackets, and the tail portion follows (is to the right of) the brackets. For example, in the compound c[EsK]WsC, c[EsK] is the cyclic peptide portion, and WsC is the peptide tail portion. As another example, in c[EsK]W4C, the cyclic peptide portion is c[EsK] and the peptide tail portion is W4C.

With respect to cyclic peptides, the amino acids within brackets may be present in the order listed in brackets from left to right, or in any order. For example, a cyclic peptide c[X2Y2] may have the corresponding linear sequence: XXYY, XYXY, YXXY, XYYX, or YXYX. In some cases, multiple examples of corresponding linear sequences for an exemplary cyclic peptide are listed in Table 3.

Table 8 provides a summary of peptide sequences.

TABLE 8
provides a summary of peptide sequences

| Peptide | Sequence | Linear Sequence | SEQ ID NO |
|---|---|---|---|
| 1 | c[(WE)₃WC] | WEWEWEWC | 228 |
| 2 | c[(WE)₄WC] | WEWEWEWEWC | 229 |
| 3 | c[(WE)₅WC] | WEWEWEWEWEWC | 230 |
| 4 | c[(LE)₄WC] | LELELELEWC | 231 |
| 5 | c[E₄W₅C] | EEEEWWWWWC | 232 |
| 6 | l(CW(EW)₄) | CWEWEWEWEW | 233 |
| 7 | c[R₄W₅C] | RRRRWWWWWC | 234 |

Table 9 provides additional non-limiting examples of peptide sequences.

| Cy- | | | |
|---|---|---|---|
| clic | | | |
| Pep- | Circular | Linear | |
| tide | Sequence | Sequence | SEQ ID NO |
| 1 | c[E₃W₅C] | EEEWWWWWC | 235 |
| 2 | c[E₃W₅C] | EWEWWWWEC | 236 |
| 3 | c[E₃W₅C] | EWWEWWWEC | 237 |
| 4 | c[E₃W₅C] | EWWWEWWEC | 238 |
| 5 | c[E₃W₅C] | EWWWWEWEC | 239 |
| 6 | c[E₃W₅C] | EWWWWWEEC | 240 |
| 7 | c[E₃W₅C] | EWEEWWWWC | 241 |
| 8 | c[E₃W₅C] | EWWEEWWWC | 242 |
| 9 | c[E₃W₅C] | EWWWEEWWC | 243 |
| 10 | c[E₃W₅C] | EWWWWEEWC | 244 |
| 11 | c[E₃W₅C] | WEEEWWWWC | 245 |
| 12 | c[E₃W₅C] | WWEEEWWWC | 246 |
| 13 | c[E₃W₅C] | WWWEEEWWC | 247 |
| 14 | c[E₃W₅C] | WWWWEEEWC | 248 |
| 15 | c[E₃W₅C] | WEWEEWWWC | 249 |
| 16 | c[E₃W₅C] | WEWWEEWWC | 250 |
| 17 | c[E₃W₅C] | WEWWWEEWC | 251 |
| 18 | c[E₃W₅C] | WEWWWWEEC | 252 |
| 19 | c[E₃W₅] | EEEWWWWW | 253 |
| 20 | c[E₃W₅] | EWEWWWWE | 254 |
| 21 | c[E₃W₅] | EWWEWWWE | 255 |
| 22 | c[E₃W₅] | EWWWEWWE | 256 |
| 23 | c[E₃W₅] | EWWWWEWE | 257 |
| 24 | c[E₃W₅] | EWWWWWEE | 258 |
| 25 | c[E₃W₅] | EWEEWWWW | 259 |
| 26 | c[E₃W₅] | EWWEEWWW | 260 |
| 27 | c[E₃W₅] | EWWWEEWW | 261 |
| 28 | c[E₃W₅] | EWWWWEEW | 262 |
| 29 | c[E₃W₅] | WEEEWWWW | 263 |
| 30 | c[E₃W₅] | WWEEEWWW | 264 |
| 31 | c[E₃W₅] | WWWEEEWW | 265 |
| 32 | c[E₃W₅] | WWWWEEEW | 266 |
| 33 | c[E₃W₅] | WEWEEWWW | 267 |
| 34 | c[E₃W₅] | WEWWEEWW | 268 |
| 35 | c[E₃W₅] | WEWWWEEW | 269 |
| 36 | c[E₃W₅] | WEWWWWEE | 270 |
| 37 | c[D₃W₅C] | DDDWWWWWC | 271 |
| 38 | c[D₃W₅C] | DWDWWWWDC | 272 |
| 39 | c[D₃W₅C] | DWWDWWWDC | 273 |
| 40 | c[D₃W₅C] | DWWWDWWDC | 274 |
| 41 | c[D₃W₅C] | DWWWWDWDC | 275 |
| 42 | c[D₃W₅C] | DWWWWWDDC | 276 |
| 43 | c[D₃W₅C] | DWDDWWWWC | 277 |
| 44 | c[D₃W₅C] | DWWDDWWWC | 278 |
| 45 | c[D₃W₅C] | DWWWDDWWC | 279 |
| 46 | c[D₃W₅C] | DWWWWDDWC | 280 |
| 47 | c[D₃W₅C] | WDDDWWWWC | 281 |
| 48 | c[D₃W₅C] | WWDDDWWWC | 282 |
| 49 | c[D₃W₅C] | WWWDDDWWC | 283 |
| 50 | c[D₃W₅C] | WWWWDDDWC | 284 |
| 51 | c[D₃W₅C] | WDWDDWWWC | 285 |
| 52 | c[D₃W₅C] | WDWWDDWWC | 286 |
| 53 | c[D₃W₅C] | WDWWWDDWC | 287 |
| 54 | c[D₃W₅C] | WDWWWWDDC | 288 |
| 55 | c[D₃W₅] | DDDWWWWW | 289 |
| 56 | c[D₃W₅] | DWDWWWWD | 290 |
| 57 | c[D₃W₅] | DWWDWWWD | 291 |
| 58 | c[D₃W₅] | DWWWDWWD | 292 |
| 59 | c[D₃W₅] | DWWWWDWD | 293 |
| 60 | c[D₃W₅] | DWWWWWDD | 294 |
| 61 | c[D₃W₅] | DWDDWWWW | 295 |
| 62 | c[D₃W₅] | DWWDDWWW | 296 |
| 63 | c[D₃W₅] | DWWWDDWW | 297 |
| 64 | c[D₃W₅] | DWWWWDDW | 298 |
| 65 | c[D₃W₅] | WDDDWWWW | 299 |
| 66 | c[D₃W₅] | WWDDDWWW | 300 |
| 67 | c[D₃W₅] | WWWDDDWW | 301 |
| 68 | c[D₃W₅] | WWWWDDDW | 302 |
| 69 | c[D₃W₅] | WDWDDWWW | 303 |
| 70 | c[D₃W₅] | WDWWDDWW | 304 |
| 71 | c[D₃W₅] | WDWWWDDW | 305 |
| 72 | c[D₃W₅] | WDWWWWDD | 306 |
| 73 | c[Gla₃W₅] | GlaGlaGlaWWWWW | 307 |
| 74 | c[Gla₃W₅] | GlaWGlaWWWWGla | 308 |
| 75 | c[Gla₃W₅] | GlaWWGlaWWWGla | 309 |
| 76 | c[Gla₃W₅] | GlaWWWGlaWWGla | 310 |
| 77 | c[Gla₃W₅] | GlaWWWWGlaWGla | 311 |
| 78 | c[Gla₃W₅] | GlaWWWWWGlaGla | 312 |
| 79 | c[Gla₃W₅] | GlaWGlaGlaWWWW | 313 |
| 80 | c[Gla₃W₅] | GlaWWGlaGlaWWW | 314 |
| 81 | c[Gla₃W₅] | GlaWWWGlaGlaWW | 315 |
| 82 | c[Gla₃W₅] | GlaWWWWGlaGlaW | 316 |
| 83 | c[Gla₃W₅] | WGlaGlaGlaWWWW | 317 |
| 84 | c[Gla₃W₅] | WWGlaGlaGlaWWW | 318 |
| 85 | c[Gla₃W₅] | WWWGlaGlaGlaWW | 319 |
| 86 | c[Gla₃W₅] | WWWWGlaGlaGlaW | 320 |
| 87 | c[Gla₃W₅] | WGlaWGlaGlaWWW | 321 |
| 88 | c[Gla₃W₅] | WGlaWWGlaGlaWW | 322 |
| 89 | c[Gla₃W₅] | WGlaWWWGlaGlaW | 323 |
| 90 | c[Gla₃W₅] | WGlaWWWWGlaGla | 324 |
| 91 | c[E₃W₄C] | EEEWWWWC | 325 |
| 92 | c[E₃W₄C] | EWEWWWEC | 326 |
| 93 | c[E₃W₄C] | EWWEWWEC | 327 |
| 94 | c[E₃W₄C] | EWWWEWEC | 328 |
| 95 | c[E₃W₄C] | EWWWWEEC | 329 |
| 96 | c[E₃W₄C] | EWEEWWWC | 330 |
| 97 | c[E₃W₄C] | EWWEEWWC | 331 |
| 98 | c[E₃W₄C] | EWWWEEWC | 332 |
| 99 | c[E₃W₄C] | EWWWWEEC | 333 |
| 100 | c[E₃W₄C] | WEEEWWWC | 334 |
| 101 | c[E₃W₄C] | WWEEEWWC | 335 |
| 102 | c[E₃W₄C] | WWWEEEWC | 336 |
| 103 | c[E₃W₄C] | WWWWEEEC | 337 |
| 104 | c[E₃W₄C] | WEWEEWWC | 338 |
| 105 | c[E₃W₄C] | WEWWEEWC | 339 |
| 106 | c[E₃W₄C] | WEWWWEEC | 340 |
| 107 | c[E₃W₄] | EEEWWWW | 341 |
| 108 | c[E₃W₄] | EWEWWWE | 342 |
| 119 | c[E₃W₄] | EWWEWWE | 343 |
| 110 | c[E₃W₄] | EWWWEWE | 344 |
| 111 | c[E₃W₄] | EWWWWEE | 345 |
| 112 | c[E₃W₄] | EWEEWWW | 346 |
| 113 | c[E₃W₄] | EWWEEWW | 347 |
| 114 | c[E₃W₄] | EWWWEEW | 348 |
| 115 | c[E₃W₄] | EWWWWEE | 349 |
| 116 | c[E₃W₄] | WEEEWWW | 350 |
| 117 | c[E₃W₄] | WWEEEWW | 351 |
| 118 | c[E₃W₄] | WWWEEEW | 352 |
| 119 | c[E₃W₄] | WWWWEEE | 353 |
| 120 | c[E₃W₄] | WEWEEWW | 354 |
| 121 | c[E₃W₄] | WEWWEEW | 355 |
| 122 | c[E₃W₄] | WEWWWEE | 356 |
| 123 | c[D₃W₄C] | DDDWWWWC | 357 |
| 124 | c[D₃W₄C] | DWDWWWDC | 358 |
| 125 | c[D₃W₄C] | DWWDWWDC | 359 |
| 126 | c[D₃W₄C] | DWWWDWDC | 360 |
| 127 | c[D₃W₄C] | DWWWWDDC | 361 |
| 128 | c[D₃W₄C] | DWDDWWWC | 362 |
| 129 | c[D₃W₄C] | DWWDDWWC | 363 |
| 130 | c[D₃W₄C] | DWWWDDWC | 364 |
| 131 | c[D₃W₄C] | DWWWWDDC | 365 |
| 132 | c[D₃W₄C] | WDDDWWWC | 366 |
| 133 | c[D₃W₄C] | WWDDDWWC | 367 |
| 134 | c[D₃W₄C] | WWWDDDWC | 368 |
| 135 | c[D₃W₄C] | WWWWDDDC | 369 |
| 136 | c[D₃W₄C] | WDWDDWWC | 370 |
| 137 | c[D₃W₄C] | WDWWDDWC | 371 |
| 138 | c[D₃W₄C] | WDWWWDDC | 372 |
| 139 | c[D₃W₄] | DDDWWWW | 373 |
| 140 | c[D₃W₄] | DWDWWWD | 374 |
| 141 | c[D₃W₄] | DWWDWWD | 375 |
| 142 | c[D₃W₄] | DWWWDWD | 376 |
| 143 | c[D₃W₄] | DWWWWDD | 377 |
| 144 | c[D₃W₄] | DWDDWWW | 378 |
| 145 | c[D₃W₄] | DWWDDWW | 379 |
| 146 | c[D₃W₄] | DWWWDDW | 380 |
| 147 | c[D₃W₄] | DWWWWDD | 381 |
| 148 | c[D₃W₄] | WDDDWWW | 382 |
| 149 | c[D₃W₄] | WWDDDWW | 383 |
| 150 | c[D₃W₄] | WWWDDDW | 385 |
| 151 | c[D₃W₄] | WWWWDDD | 386 |
| 152 | c[D₃W₄] | WDWDDWW | 387 |
| 153 | c[D₃W₄] | WDWWDDW | 388 |
| 154 | c[D₃W₄] | WDWWWDD | 399 |
| 155 | c[Gla₃W₄] | GlaGlaGlaWWWW | 400 |
| 156 | c[Gla₃W₄] | GlaWGlaWWWGla | 401 |
| 157 | c[Gla₃W₄] | GlaWWGlaWWGla | 402 |
| 158 | c[Gla₃W₄] | GlaWWWGlaWGla | 403 |
| 159 | c[Gla₃W₄] | GlaWWWWGlaGla | 404 |
| 160 | c[Gla₃W₄] | GlaWGlaGlaWWW | 405 |
| 161 | c[Gla₃W₄] | GlaWWGlaGlaWW | 406 |
| 162 | c[Gla₃W₄] | GlaWWWGlaGlaW | 407 |
| 163 | c[Gla₃W₄] | GlaWWWWGlaGla | 408 |
| 164 | c[Gla₃W₄] | WGlaGlaGlaWWW | 409 |
| 165 | c[Gla₃W₄] | WWGlaGlaGlaWW | 410 |
| 166 | c[Gla₃W₄] | WWWGlaGlaGlaW | 411 |
| 167 | c[Gla₃W₄] | WWWWGlaGlaGla | 412 |
| 168 | c[Gla₃W₄] | WGlaWGlaGlaWW | 413 |
| 169 | c[Gla₃W₄] | WGlaWWGlaGlaW | 414 |
| 170 | c[Gla₃W₄] | WGlaWWWGlaGla | 415 |
| 171 | c[(WE)₃WC] | WEWEWEWC | 416 |
| 172 | c[(EW)₃WC] | EWEWEWWC | 417 |
| 173 | c[(WD)₃WC] | WDWDWDWC | 418 |
| 174 | c[(DW)₃WC] | DWDWDWWC | 419 |
| 175 | c[(WGla)₃WC] | WGlaWGlaWDWC | 420 |
| 176 | c[(GlaW)₃WC] | DWDWDWDC | 421 |
| 177 | c[(WE)₄] | WEWEWEWE | 422 |
| 178 | c[(EW)₄] | EWEWEWEW | 423 |
| 179 | c[(WD)₄] | WDWDWDWD | 424 |
| 180 | c[(DW)₄] | DWDWDWDW | 425 |
| 181 | c[(WGla)₄] | WGlaWGlaWGlaWGla | 426 |
| 182 | c[(GlaW)₄] | GlaWGlaWGlaWGlaW | 427 |
| 183 | c[CW(EW)₄] | CWEWEWEWEW | 428 |
| 184 | c[(WGla)₂WDWC] | WGlaWGlaWDWC | 429 |
| 185 | c[(EW)₃EC] | EWEWEWEC | 430 |
| 186 | c[(DW)₃DC] | DWDWDWDC | 431 |
| 187 | c[E₅K]W₅C | Cyclic: EEEEEK | 432 (cyclic portion), |
| | | Tail: WWWWWC | 433 (Tail) |
| 188 | c[E₄K]W₅C | Cyclic: EEEEK | 434 (cyclic portion), |
| | | Tail: WWWWWC | 435 (Tail) |
| 189 | c[E₅K]W₄C | Cyclic: EEEEEK | 436 (cyclic portion), |
| | | Tail: WWWWC | 437 (Tail) |
| 190 | c[E₄K]W₄C | Cyclic: EEEEK | 438 (cyclic portion), |
| | | Tail: WWWWC | 439 (Tail) |
| 191 | c[E₅K]W₅ | Cyclic: EEEEEK | 440 (cyclic portion), |
| | | Tail: WWWWW | 441 (Tail) |
| 192 | c[E₄K]W₅ | Cyclic: EEEEK | 442 (cyclic portion), |
| | | Tail: WWWWW | 443 (Tail) |
| 193 | c[E₅K]W₄ | Cyclic: EEEEEK | 444 (cyclic portion), |
| | | Tail: WWWW | 445 (Tail) |
| 194 | c[E₄K]W₄ | Cyclic: EEEEK | 446 (cyclic portion), |
| | | Tail: WWWW | 447 (Tail) |
| 195 | c[D₅K]W₅C | Cyclic: DDDDDK | 448 (cyclic portion), |
| | | Tail: WWWWWC | 449 (Tail) |
| 196 | c[D₄K]W₅C | Cyclic: DDDDK | 450 (cyclic portion), |
| | | Tail: WWWWWC | 451 (Tail) |
| 197 | c[D₅K]W₄C | Cyclic: DDDDDK | 452 (cyclic portion), |
| | | Tail: WWWWC | 453 (Tail) |
| 198 | c[D₄K]W₄C | Cyclic: DDDDK | 454 (cyclic portion), |
| | | Tail: WWWWC | 455 (Tail) |
| 199 | c[D₅K]W₅ | Cyclic: DDDDDK | 456 (cyclic portion), |
| | | Tail: WWWWW | 457 (Tail) |
| 200 | c[D₄K]W₅ | Cyclic: DDDDK | 458 (cyclic portion), |
| | | Tail: WWWWW | 459 (Tail) |
| 201 | c[D₅K]W₄ | Cyclic: DDDDDK | 460 (cyclic portion), |
| | | Tail: WWWW | 461 (Tail) |
| 202 | c[D₄K]W₄ | Cyclic: DDDDK | 462 (cyclic portion), |
| | | Tail: WWWW | 463 (Tail) |
| 203 | c[Gla₅K]W₅C | Cyclic: GlaGla | 464 (cyclic portion), |
| | | GlaGlaGlaK | 465 (Tail) |
| | | Tail: WWWWWC | |
| 204 | c[Gla₄K]W₅C | Cyclic: GlaGla | 466 (cyclic portion), |
| | | GlaGlaK | 467 (Tail) |
| | | Tail: WWWWWC | |
| 205 | c[Gla₅K]W₄C | Cyclic: GlaGla | 468 (cyclic portion), |
| | | GlaGlaGlaK | 469 (Tail) |
| | | Tail: WWWWC | |
| 206 | c[Gla₄K]W₄C | Cyclic: GlaGla | 470 (cyclic portion), |
| | | GlaGlaK | 471 (Tail) |
| | | Tail: WWWWC | |
| 207 | c[Gla₅K]W₅ | Cyclic: GlaGla | 472 (cyclic portion), |
| | | GlaGlaGlaK | 473 (Tail) |
| | | Tail: WWWWW | |
| 208 | c[Gla₄K]W₅ | Cyclic: GlaGla | 474 (cyclic portion), |
| | | GlaGlaK | 475 (Tail) |
| | | Tail: WWWWW | |
| 209 | c[Gla₅K]W₄ | Cyclic: GlaGla | 476 (cyclic portion), |
| | | GlaGlaGlaK | 477 (Tail) |
| | | Tail: WWWW | |
| 210 | c[Gla₄K]W₄ | Cyclic: GlaGla | 478 (cyclic portion), |
| | | GlaGlaK | 479 (Tail) |
| | | Tail: WWWW | |
| 211 | c[E₅W₅C] | EEEEEWWWWWC | 480 |
| 212 | c[E₄W₄C] | EEEEWWWWC | 481 |
| 213 | c[(WE)₄CW] | WEWEWEWECW | 482 |
| 214 | c[(WR)₄WC] | WRWRWRWRWC | 483 |

### Production of Epitope-pHLIP® Peptide Compositions

To manufacture the constructs or compositions to decorate the cell surfaces of diseased cell, a variety of methods known in the art can be used, e.g.:
i) epitope synthesized and linked to the membrane non-inserting part of pHLIP® peptide; or
ii) epitope synthesized as membrane non-inserting part of pHLIP® as a single peptide; or
iii) epitope expressed as membrane non-inserting part of pHLIP® fusion protein.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Tethering Small Molecule Epitope Di-Nitrophenyl (DNP) to Cancer Cells by pHLIP® Promoted Cell Killing

Three different pHLIP® constructs were synthesized with a DNP—(0-(2,4-dinitrophenyl)hydroxylamine):
i) DNP-pHLIP®, where DNP-malemide was conjugated with a single Cys residue at the N-terminal of the pHLIP® peptide;
ii) DNP-PEG4-pHLIP®, where DNP-PEG4-NHS was conjugated with a single Lys residue at the N-terminal of the pHLIP® peptide; and
iii) DNP-PEG12-pHLIP®, where DNP-PEG12-NHS as conjugated with a single Lys residue at the N-terminal of pHLIP® peptide.

pHLIP® peptide with a single Cys residues used in the study for conjugation with DNP-malemide is the following: (ACDDQNPWRAYLDLLFPTDTLLLDLLWA (SEQ ID NO: 509) pHLIP® peptide with single Lys residue and acetylated N-terminus used in the study for conjugation with DNP-PEG4-NHS and DNP-PEG12-NHS is the following: Ac-AKDDQNPWRAYLDLLFPTDTLLLDLLWA (SEQ ID NO: 510)). Peptides were prepared by solid-phase synthesis. Progressions of coupling reactions and purifications were performed using reverse-phase HPLC (RP-HPLC) (the gradient: water and acetonitrile with 0.05% trifluoroacetic acid (TFA) followed by lyophilization. The purity and identity of the constructs were established by analytical RP-HPLC and surface-enhanced laser desorption/ionization time of flight (SELDI-TOF) mass spectroscopy, respectively. Constructs concentrations were calculated by absorbance at 280 nm using pHLIP® peptide extinction coefficient.

The key for induction of immunological response is a proper positioning of epitope at the surface of tumor cells, which was verified on 3-D tumor cancer cell culture (tumor spheroids). Briefly, a 2% agarose solution was made by dissolving in pH 7.4 PBS. 150 μL of the solution was pipetted into each well of a 48-well flat bottom tissue culture plate. After the agarose gel sufficiently settled (˜1 h), 150 μL of DMEM supplemented with 10% FBS and ciprofloxacin-HCl was added to each well. The covered plate was left in a humidified atmosphere at 37° C. and 5% CO₂ in cell culture incubator for 24 h. On the next day, the excess medium was removed from the agarose layer. HeLa cells (10,000 cells) in 200 μL of DMEM containing 2% matrigel were added into each well and incubated for 3-4 days to allow the formation of spheroids. Matrigel was dissolved on ice overnight and added in ice cold DMEM at a concentration of 2.5% (to obtain a final concentration of 2% once added to the wells). Then the mixture was heated to 37° C. before being combined with the cells. Tumor spheroids were incubated in 50 μL of PBS buffer, pH 6.0-6.5 containing 0-2 μM DNP-pHLIP®, DNP-PEG4-pHLIP® or DNP-PEG12-pHLIP® in a humidified atmosphere of 5% CO₂ at 37° C. for 30 min. After treatment, the spheroids were washed several times in 1 mL of PBS. Next, spheroids were treated with anti-DNP antibody labeled with 647 nm fluorescent dye at pH 7.4 followed by washing. Spheroids were also stained with DAPI to mark cell nucleus.

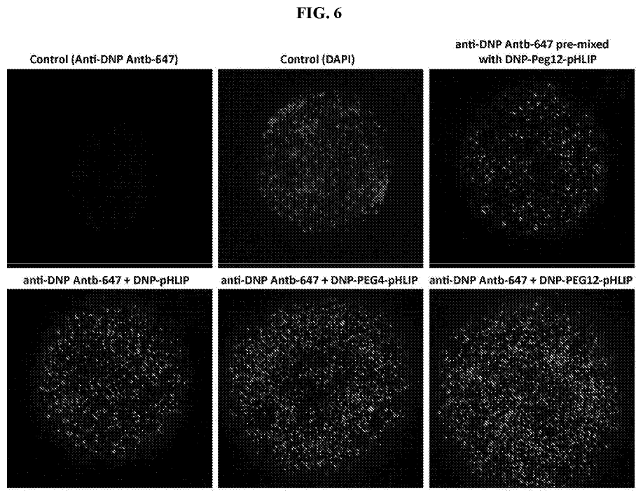

The spheroids were imaged using a fluorescent inverted confocal microscope. The representative images are shown in FIG. 6. The data clearly indicate that pHLIP® peptide positioned DNP epitope at the surface of cancer cells in 3-D cell culture, and the epitope was recognized by the corresponding antibody.

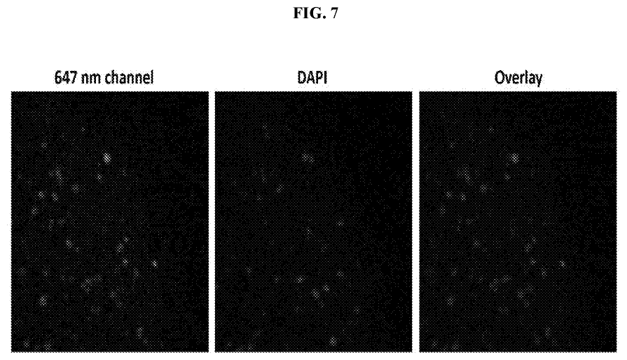

Images presented in FIG. 7 were obtained at high magnification to demonstrate that fluorescent signal from the antibody coincided with the fluorescent signal from DAPI, which stains DNA in cell nucleus. There were multiple focal planes in 3-D tumor spheroid and focus for DAPI signal and 647 nm fluorescent light were different, however an overlay between fluorescence from the antibody and DAPI fluorescence is clearly observed.

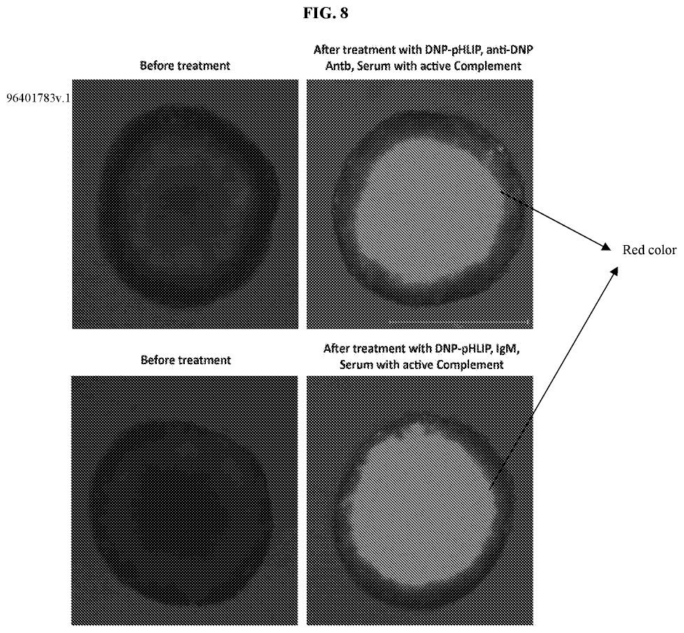

To establish a biological effect, the tumor speroids were treated with 5 sM of DNP-pHLIP®, DNP-PEG4-pHLIP® or DNP-PEG12-pHLIP® for 1 hour in PBS pH6.5, washed followed by incubation with anti-DNP antibody or human IgM antibody for 1 hr in PBS pH7.4. Then human serum with active complement and propidium iodine (PI) were added, washed and spheroids were imaged. Cell impermeable PI dye stains only dead (or dying) cells with the compromised membrane. Therefore presence of red color on FIG. 8 indicated that plasma membrane of cells was comrpomized by complement-mediated attack and that the cells were dying.

### Example 2: Tethering Two Peptide Epitopes by pHLIP® to Cancer Cells to Bind Two Heads of Ig Antibody

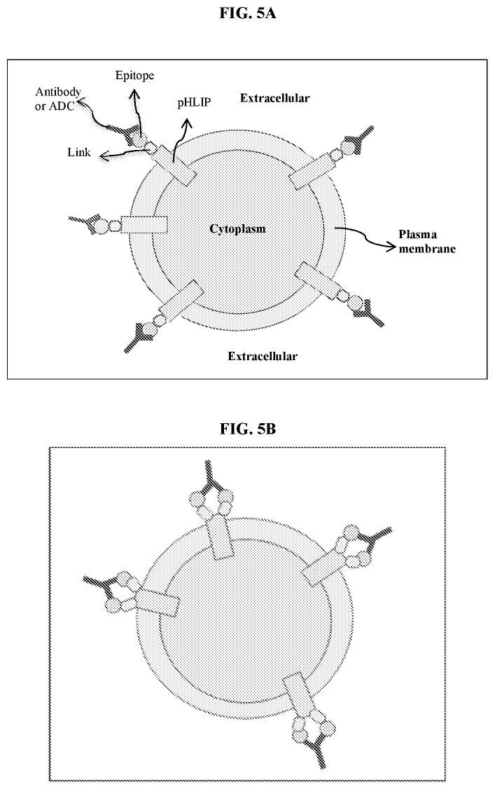

To enhance performance of antibodies and enhance immune response, it is important to promote binding of both heads of IgG with 2 epitopes coupled to the same pHLIP® peptide (see, e.g., FIG. 5B). The pHLIP® peptide with 2 Lys residues (bold and underlined) (Ac-AKQNDDQNKPWRAYLDLLFPTDTLLLDLLWA (SEQ ID NO: 511)) is conjugated with excess of NHS-PEG12-malemide and NHS-PEG24-malemide linkers, purified and, pHLIP-(PEG12)2 is coupled with an HA peptide epitope (YPYDVPDYAGGGCA (SEQ ID NO: 6)). pHLIP® and HA peptides are prepared by solid-phase synthesis. Progressions of both coupling reactions and purifications are performed using reverse-phase HPLC (RP-HPLC) (the gradient: water and acetonitrile with 0.05% TFA) followed by lyophilization. The concentration of the construct is measured by absorbance at 280 nm.

PEG12 and PEG24 are be stretched for 5 nm and 10 nm, respectively. The six residues (QNDDQN (SEQ ID NO: 541)) between points of PEG conjugation to pHLIP® provides additional space of few nanometers, for example, from 5 to 25 nm, or, from about 10-15 nm. Thus, two epitopes at the single pHLIP® construct bind two heads of Ig antibody, since the distance between heads is 10-15 nm.

HeLa cancer cells in 2D and 3D cell culture are stained with (HA)2-(PEG12)2-pHLIP®, (HA)₂-(PEG24)2-pHLIP®, HA-PEG12-pHLIP® and HA-PEG24-pHLIP®. Affinity of fluorescent antibody against HA peptide epitope is evaluated.

### Example 3: Tethering CXCL10 Protein Chemokine Epitope by pHLIP® to Cancer Cells to Promote NK-Cells Binding

Two fusion proteins with 2 different tags (His and cMyc) are expressed and purified:

CXCL10-mucin-2×-Myc-pHLIP®

SEQ ID NO: 531
mnqtailicclifltlsgiqgvplsrtvrctcisisnqpvnprslekleii
pasqfcprveiiatmkkkgekrclnpeskaiknllkavskerskrspgtfe
kqigevkprttpaaggmdesvvlepeatgesssleptpssqeaqralgtsp
elptgvtgssgtrlpptpkaqdggpvgtelfrvppvstaatwqssaphqpg
pslwaeaktseapstqdpstqastasspapeenapsegqrvwgqgqsprpe
nslereemgpvpahtdafqdwgpgsmahvsvvpvssegtpsrepvasgswt
pkaeepihatmdpqrlgvlitpvpdaqaatrrqeqkliseedleqklisee
dladdqnpwrayidllfptdtllldllw

CXCL10-mucin-6×-His-pHLIP®

SEQ ID NO: 532
mnqtailicclifltlsgiggvplsrtvrctcisisnqpvnprslekleii
pasqfcprveiiatmkkkgekrclnpeskaiknllkavskerskrspgtfe
kqigevkprttpaaggmdesvvlepeatgesssleptpssqeagralgtsp
elptgvtgssgtrlpptpkaqdggpvgtelfrvppvstaatwqssaphqpg
pslwaeaktseapstqdpstqastasspapeenapsegqrvwgqgqsprpe
nslereemgpvpahtdafqdwgpgsmahvsvvpvssegtpsrepvasgswt
pkaeepihatmdpqrlgvlitpvpdagaatrrqhhhhhhaddqnpwrayld
llfptdtllldllw

Both fusion proteins are treated with HeLa or HeLa-GFP cancer cells at pH 6.0-6.5 followed by washing and applying NK-cells loaded with red fluorescent dye DiI (1,1′-dioctadecyl-3,3,3′,3′-tetramethylindocarbocyanine) for 30 min followed by gentle washing. Binding and adhesion of NK-cells to cancer cells decorated with CXCL10 chemokine pHLIP® constructs and non-decorated are compared under fluorescent microscope by analyzing green and red fluorescent signals coming from cancer cells and immune NK-cells, respectively.

pHLIP® Peptide-Mediated Epitope Tethering at Cell Surfaces: Summary I

The invention features compositions and methods for eliciting an immune response in a subject by administering to the subject a pHLIP® construct comprising an antibody recruiting molecule or an immune cell recruiting molecule. The antibody recruiting molecule or an immune cell recruiting molecule is linked to one or more pHLIP® peptides and wherein The construct increases the amount of antibody recruiting molecule or immune cell recruiting molecule on the surface of a diseased cell.

For example, the composition comprises the formula of:

Epitope-Linker-Pept
wherein “Epitope” is an antibody or immune cell recruiting molecule;
wherein “Linker” is a non-cleavable linker compound or a membrane non-inserting end of the pHLIP® peptide further comprises an amino acid extension;
wherein “Pept” is a pHLIP® peptide comprising the sequence AXDDQNPWRAYLDLLFPTDTLLLDLLW (SEQ ID NO: 533) or AXDQDNPWRAYLDLLFPTDTLLLDLLW (SEQ ID NO: 534), where “X” is a functional group, selected from a lysine, a cysteine, or an Azido-containing amino acid;
wherein each “—” is a covalent bond;

In some examples, the construct comprises an antibody recruiting molecule; in other examples, the construct comprises an immune cell recruiting molecule. Optionally, the construct comprises both an antibody recruiting molecule or an immune cell recruiting molecule

In some embodiments, 2 antibody recruiting molecules are linked to pHLIP® peptide. Such an exemplary construct and method is described below. For example, the composition or method includes a construct that comprises the formula of

Epitope1-Linker-Pept-Linker-Epitope1
wherein “Epitope1” is an antibody recruiting molecule;
wherein “Linker” is a polyethylene glycol linker;
wherein “Pept” is a pHLIP® peptide comprising the sequence

(SEQ ID NO: 511)
Ac-AKQNDDQNKPWRAYLDLLFPTDTLLLDLLWA
or
(SEQ ID NO: 535)
Ac-AKQNDNDNKPWRAYLDLLFPTDTLLLDLLWA
or
(SEQ ID NO: 536)
ACQNDDQNCPWRAYLDLLFPTDTLLLDLLWA
or
(SEQ ID NO: 505)
ACQNDNDNCPWRAYLDLLFPTDTLLLDLLWA

wherein each “—” is a covalent bond.

As described above, exemplary recruiting molecules include one or more epitopes. For example, the epitope comprises a peptide with a length less than 50 amino acids.

The method of claim 1, wherein said antibody recruiting molecule or immune cell recruiting molecule comprises an epitope. In examples, the epitope comprises a peptide with a length less than 50 amino acids, e.g., the epitope comprises a length of between 5 to 20 amino acids. An exemplary epitope comprises an HA peptide. For example, the peptide comprises the amino acid sequence of YPYDVPDYA (SEQ ID NO: 6). Additional examples of epitopes include QVSHWVSGLAEGSFG (SEQ ID NO: 1), LSHTSGRVEGSVSLL (SEQ ID NO: 2), QMWAPQWGPD (SEQ ID NO: 3); MASMTGGQQMG (SEQ ID NO: 4); EQKLISEEDL (SEQ ID NO: 5); YTDIEMNRLGK (SEQ ID NO: 7); KETAAAKFERQHMDS (SEQ ID NO: 8); GKPIPNPLLGLDST (SEQ ID NO: 9); DYKDDDDK (SEQ ID NO: 10); GAPVPYPDPLEPR (SEQ ID NO: 11); HHHHHH (SEQ ID NO: 12); TKENPRSNQEESYDDNES (SEQ ID NO: 13); WSHPQFEK (SEQ ID NO: 14); or PDRVRAVSHWSS (SEQ ID NO: 15).

In examples, an epitope comprises a protein epitope with a length of 200 or less amino acids. For example, the protein epitope comprises a cytokine such as an interleukin (IL), e.g., IL-1, IL-2, IL-6, IL-7, IL-12, or IL-17. In some embodiments, the cytokine comprises tumor necrosis factor (TNF). In some some embodiments, the cytokine comprises a chemokine (CXC). Examples of chemokines include CXCL9, CXL10, or CXL11. For example, the chemokine comprises CXCL10 comprises the amino acid sequence:

(SEQ ID NO: 514)
MNQTAILICCLIFLTLSGIQGVPLSRTVRCTCISISNQPVNPRSLEKLEII
PASQFCPRVEIIATMKKKGEKRCLNPESKAIKNLLKAVSKERSKRSP.

In yet other examples, the epitope comprises a small molecule. For example, the small molecule comprises a dinitrophenyl (DNP) or a derivative thereof.

The methods are useful to methods for eliciting an immune response in a subject. This clinically beneficial effect is accomplished by the pHLIP® construct that increases the amount of antibody recruiting molecule or immune cell recruiting molecule on the surface of a diseased cell. For example, the diseased cell comprises a tumor cell. In another example, the diseased cell comprises a cell in inflamed tissue.

Also within the invention is a composition comprising an antibody or immune cell recruiting molecule linked to one or more pHLIP® peptides by a non-cleavable linker compound. A composition comprising an epitope linked to one or more pHLIP® peptides, wherein the epitope is a protein epitope and is an extension of the non-inserting end of the pHLIP® peptide is also within the invention. For example, the non-inserting end of the pHLIP® peptide further comprises an amino acid extension, wherein the extension comprises a protein epitope. In another aspect, the invention encompasses a composition comprising an epitope linked to one or more pHLIP® peptides, wherein the epitope and the pHLIP® peptide are part of a single fusion construct.

In the methods or compositions, the pHLIP® construct comprises the formula of Epitope-Linker-Peptide, wherein Peptide is a pHLIP® peptide comprising the sequence AXDDQNPWRAYLDLLFPTDTLLLDLLWA (SEQ ID NO: 18) or AXDQDNPWRAYLDLLFPTDTLLLDLLWA (SEQ ID NO: 495), where “X” is a functional group, selected from a lysine, a cysteine, or an Azido-containing amino acid, wherein Linker is a linker or an extension of the pHLIP® peptide, and wherein each “—” is a covalent bond.

An exemplary composition comprises the formula of Epitope-Linker-Peptide, wherein Peptide is a pHLIP® peptide comprising the sequence AXDDQNPWRAYLDLLFPTDTLLLDLLWA (SEQ ID NO: 18) or AXDQDNPWRAYLDLLFPTDTLLLDLLWA (SEQ ID NO: 495, where X is a functional group, selected from a lysine, a cysteine, or an Azido-containing amino acid, and wherein Linker is a linker or an extension of the pHLIP® peptide, and wherein each “—” is a covalent bond. In some examples, two epitopes are linked to a single pHLIP® peptide.

As described above, the construct may comprise the formula of Epitope2-Linker2-Peptide, wherein Peptide is a pHLIP® peptide comprising the sequence AX(Z)nXPWRAYLDLLFPTDTLLLDLLWA (SEQ ID NO: 496), wherein X is a functional group, selected from a lysine, a cysteine, an Azido-containing amino acid, or others, wherein Z comprises indicates any amino acid residue, wherein n is any integer between 1 and 10, wherein Linker is a linker or an extension of the pHLIP® peptide, and each “—” is a covalent bond. In another example, the composition or construct comprises the formula of Epitope2-Linker2-Pept, wherein “Pept” is a pHLIP® peptide comprising the sequence AX(Z)nXPWRAYLDLLFPTDTLLLDLLWA (SEQ ID NO: 496), where X is a functional group, selected from a lysine, a cysteine, an Azido-containing amino acid, wherein Z indicates any amino acid residue, wherein n is any integer between and including 1 and 10, wherein Linker is a linker or an extension of the pHLIP® peptide, and wherein each “—” is a covalent bond.

A method of inducing an immune response in a diseased tissue in a subject, which method comprises the step of administering to a subject a composition comprising an epitope and a pHLIP® peptide is also within the invention. For example, the subject comprises a solid tumor or the subject comprises an inflamed tissue. The pHLIP® compositions or constructs are administered in a variety of clinically-acceptable methods, e.g, the composition is injected directly into a diseased tissue tumor mass. In another example, the composition is systemically administered. The method is associated with numerous advantages, e.g., the biological effect of the composition in eliciting or promoting an antigen-specific (epitope-specific) immune response is at least 20% greater than that delivered in the absence of said composition. The response may be at least 25, 50, 75, 90% and even 2-fold, 3-fold, 5-fold, 10-fold or more greater than that delivered in the absence of pHLIP® composition or construct. Another significant advantage is that the composition targets preferentially to a diseased tissue compared to a healthy tissue, thereby minimizing damage to said healthy tissue.

Also within the invention is a method for promoting an immune response in a subject, comprising administering to a subject the pHLIP® compositions and constructs described herein, wherein the method comprises placement of the epitope on tumor cell or a cell in inflamed tissue of said subject.

pHLIP® Peptide-Mediated Epitope Tethering at Cell Surfaces: Summary II

Exemplary pHLIP® compositions comprise an epitope and a pHLIP® peptide.

In one example, the composition comprises the formula of

Epitope-Linker-Pept
wherein “Epitope” is an antibody or immune cell recruiting molecule;
wherein “Linker” is a non-cleavable linker compound or a membrane non-inserting end of the pHLIP® peptide further comprises an amino acid extension;
wherein “Pept” is a pHLIP® peptide comprising the sequence AXDDQNPWRAYLDLLFPTDTLLLDLLW (SEQ ID NO: 533) or AXDQDNPWRAYLDLLFPTDTLLLDLLW (SEQ ID NO: 534), where “X” is a functional group, selected from a lysine, a cysteine, or an Azido-containing amino acid;
wherein each “—” is a covalent bond.

### Antibody Recruitment

In preferred embodiments, the epitope is an antibody recruiting molecule. For example, 2 antibody recruiting molecules are linked to pHLIP® peptide. An example of such a composition for recruiting antibodies includes a composition comprising the formula of

Epitope1-Linker-Pept-Linker-Epitope1
wherein “Epitope1” is an antibody recruiting molecule;
wherein “Linker” is a polyethylene glycol linker;
wherein “Pept” is a pHLIP® peptide comprising the sequence

(SEQ ID NO: 511)
Ac-AKQNDDQNKPWRAYLDLLFPTDTLLLDLLWA
or
(SEQ ID NO: 535)
Ac-AKQNDNDNKPWRAYLDLLFPTDTLLLDLLWA
or
(SEQ ID NO: 536)
ACQNDDQNCPWRAYLDLLFPTDTLLLDLLWA
or
(SEQ ID NO: 505)
ACQNDNDNCPWRAYLDLLFPTDTLLLDLLWA

wherein each “—” is a covalent bond;

For example, an epitope comprises a peptide with a length less than 50 amino acids, e.g., a length of between 5 to 20 amino acids. An exemplary epitope comprises an HA peptide, e.g., the peptide comprises the amino acid sequence of YPYDVPDYA (SEQ ID NO: 6).

Other epitopes include the following peptides: QVSHWVSGLAEGSFG (SEQ ID NO: 1), LSHTSGRVEGSVSLL (SEQ ID NO: 2), QMWAPQWGPD (SEQ ID NO: 3); MASMTGGQQMG (SEQ ID NO: 4); EQKLISEEDL (SEQ ID NO: 5); KETAAAKFERQHMDS (SEQ ID NO: 8); GKPIPNPLLGLDST (SEQ ID NO: 9); DYKDDDDK (SEQ ID NO: 10); GAPVPYPDPLEPR (SEQ ID NO: 11); HHHHHH (SEQ ID NO: 12); TKENPRSNQEESYDDNES (SEQ ID NO: 13); WSHPQFEK (SEQ ID NO: 14); and/or PDRVRAVSHWSS (SEQ ID NO: 15).

In some examples, the epitope comprises a small molecule such as dinitrophenyl (DNP) or a derivative thereof.

### Immune Cell Recruitment

In some examples the epitope is an immune cell recruiting molecule.

An exemplary composition, e.g. for recruiting immune cells, comprises the formula of

Epitope2-Pept,
wherein “Epitope2” is an immune cell recruiting molecule;
wherein “Pept” is a pHLIP® peptide comprising the sequence

(SEQ ID NO: 179)
ADDQNPWRAYLDLLFPTDTLLLDLLW;

wherein “—” is a covalent bond. In such examples, the epitope comprises a protein epitope with a length of 350 or less amino acids. An exemplary protein epitope comprises a cytokine. For example, the cytokine comprises an interleukin (IL) such as IL-2, IL-6, IL-7, or IL-12. In other examples, the cytokine comprises tumor necrosis factor (TNF).

In some embodiments, the cytokine comprises a chemokine such as CXCL9, CXCL10, or CXCL11. For example, chemokine comprises CXCL10 comprising the amino acid sequence:

(SEQ ID NO: 514)
MNQTAILICCLIFLTLSGIQGVPLSRTVRCTCISISNQPVNPRSLEKLEII
PASQFCPRVEIIATMKKKGEKRCLNPESKAIKNLLKAVSKERSKRSP.

Compositions with Amino Acid Extension to pHLIP® Peptide

In some aspect, the invention features compositions, constructs, and methods comprising an epitope wherein the membrane non-inserting end of the pHLIP® peptide further comprises an amino acid extension. For example, the composition comprises an epitope and pHLIP® peptide comprises a fusion protein. For example, fusion protein comprises an epitope and a pHLIP® peptide. In other examples, a composition comprises an epitope linked to one or more pHLIP® peptides by a non-cleavable linker compound.

A variety of linkers may be used. For example, the linker is a chemical polymer, e.g., polyethylene glycol. In other examples, the linker is a biopolymer. Exemplary linkers include mucin domain, dextran, cellulose, chitin or starch.

### Methods of Treatment

The invention also includes a method of inducing an immune response in a diseased tissue in a subject, comprising administering to a subject a composition comprising an epitope and a pHLIP® peptide. For example, the subject comprises a solid tumor. Alternatively, the subject comprises an inflamed tissue. In some cases, both conditions are present in the subject.

The subject is treated using a variety of clinically acceptable procedures, e.g, the composition is injected directly into a diseased tissue tumor mass. In another example, the composition is systemically administered. As described above, an advantage of the methods is that a biological effect of said composition is at least 20% or more greater than that delivered in the absence of said composition. Another advantage that contributes to the clinical safety and efficacy is that the composition targets preferentially to a diseased tissue compared to a healthy tissue, thereby minimizing damage to healthy tissue.

An exemplary method for promoting an immune response in a subject is carried out by administering to a subject the compositions and constructs described above. Such methods comprise, e.g., lead to, the placement of the epitope(s) on tumor cell or a cell in inflamed tissue of the subject. The increase amount or concentration of the epitope on the surface of the tumor cell or cell in an inflamed tissue leads to a more robust immune response, e.g., antibody-binding or immune cell binding, and subsequent killing and/or elimination of the diseased (or otherwise undesirable) cell. For example, the diseased cell comprises a tumor cell or a cell in inflamed tissue.

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. Purified also defines a degree of sterility that is safe for administration to a human subject, e.g., lacking infectious or toxic agents. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The term “alkyl,” by itself or as part of another substituent, means, unless otherwise stated, a non-cyclic straight (i.e., unbranched) or branched chain, or combination thereof, which may be fully saturated, mono- or polyunsaturated and can include di- and multivalent radicals, having the number of carbon atoms designated (i.e., C1-C10 means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH2CH2CH2CH2-. Typically, an alkyl (or alkylene) group will have from 1 to 24 carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom (e.g. selected from the group consisting of O, N, P, S, Se and Si, and wherein the nitrogen, selenium, and sulfur atoms may optionally be oxidized, and the nitrogen heteroatom may optionally be quaternized). The heteroatom(s) 0, N, P, S, Se, and Si may be placed at any interior position of the heteroalkyl group or at the position at which the alkyl group is attached to the remainder of the molecule. Examples include, but are not limited to: —CH2-CH2-O—CH3, —CH2-CH2-NH—CH3, —CH2-CH2-N(CH3)—CH3, —CH2-S-CH2-CH3, —CH2-CH2, —S(O)—CH3, —CH2-CH2-S(O)2-CH3, —CH═CH—O—CH3, —Si(CH3)3, —CH2-CH═N—OCH3, —CH═CH—N(CH3)—CH3, —O—CH3, —O-CH2-CH3, and —CN. Up to two heteroatoms may be consecutive, such as, for example, —CH2-NH—OCH3.

Similarly, the term “heteroalkylene,” by itself or as part of another substituent, means, unless otherwise stated, a divalent radical derived from heteroalkyl, as exemplified, but not limited by, —CH2-CH2-S-CH2-CH2- and —CH2-S-CH2-CH2-NH—CH2-. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)2R′— represents both —C(O)2R′- and —R′C(O)2-. As described above, heteroalkyl groups, as used herein, include those groups that are attached to the remainder of the molecule through a heteroatom, such as —C(O)R′, —C(O)NR′, —NR′R″, —OR′, —SeR′, —SR′, and/or —SO2R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A“cycloalkylene” and a “heterocycloalkylene,” alone or as part of another substituent, means a divalent radical derived from a cycloalkyl and heterocycloalkyl, respectively.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms (e.g. selected from N, O, and S, wherein the nitrogen and sulfur atoms are optionally oxidized, and the nitrogen atom(s) are optionally quaternized). Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

Each of the above terms (e.g., “alkyl,” “heteroalkyl,” “aryl,” and “heteroaryl”) includes both substituted and unsubstituted forms of the indicated radical.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

An epitope is a molecular region of an antigen capable of eliciting an immune response and of combining with a specific antibody or immune cell produced by such a response. An epitope is also know as an antigenic determinant. For example, an epitope is a part of an antigen molecule to which an antibody attaches or to which an immune cell attaches.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (cutaneous T-cell lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

By, “small molecule” may be referred to broadly as an organic, inorganic or organometallic compound with a low molecular weight compound (e.g., a molecular weight of less than about 2,000 Da or less than about 1,000 Da). The small molecule may have a molecular weight of less than about 2,000 Da, a molecular weight of less than about 1,500 Da, a molecular weight of less than about 1,000 Da, a molecular weight of less than about 900 Da, a molecular weight of less than about 800 Da, a molecular weight of less than about 700 Da, a molecular weight of less than about 600 Da, a molecular weight of less than about 500 Da, a molecular weight of less than about 400 Da, a molecular weight of less than about 300 Da, a molecular weight of less than about 200 Da, a molecular weight of less than about 100 Da, or a molecular weight of less than about 50 Da.

Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. Typically small molecules are less than one kilodalton.

As used herein, the term “stereoisomers” refers to compounds made up of the same atoms having the same bond order but having different three-dimensional arrangements of atoms that are not interchangeable. The three-dimensional structures are called configurations. As used herein, the term “enantiomers” refers to two stereoisomers that are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. In various embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A the “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared different lengths, and the comparison window is the entire length of the longer or the shorter of the two sequences. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

In various embodiments, an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information, as known in the art. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of eliciting an immune response in a subject comprising administering to the subject a pH-triggered construct comprising an antibody recruiting molecule or an immune cell recruiting molecule linked to one or more pH-triggered peptides.

2. The method of claim 1, wherein the antibody recruiting molecule or the immune cell recruiting molecule comprises an epitope.

3. The method of claim 1, wherein the construct comprises the formula of Epitope-Linker-Pept
wherein “Epitope” is an antibody recruiting molecule or immune cell recruiting molecule;
wherein “Linker” is a non-cleavable linker compound or a membrane non-inserting end of the pH-triggered peptide further comprises an amino acid extension;
wherein “Pept” is a pH-tri r peptide comprising the sequence
AXDDQNPWRAYLDLLFPTDTLLLDLLW (SEQ ID NO: 533) or
AXDQDNPWRAYLDLLFPTDTLLLDLLW (SEQ ID NO: 534), wherein upper case “X” indicates any amino acid residue, and can include a lysine, a cysteine, or an Azido-containing amino acid; and
wherein each “—” is a covalent bond.

4. (canceled)

5. The method of claim 1, wherein two antibody recruiting molecules are linked to the pH-triggered peptide.

6-12. (canceled)

13. The method of claim 2, wherein IM epitope is a HA peptide.

14. The method of claim 2, wherein the epitopes comprises the amino acid sequence of YPYDVPDYA (SEQ ID NO: 6).

15. The method of claim 2, wherein the epitope is selected from the group consisting of QVSHWVSGLAEGSFG (SEQ ID NO: 1), LSHTSGRVEGSVSLL (SEQ ID NO: 2), QMW-APQWGPD (SEQ ID NO: 3); MASMTGGQQMG (SEQ ID NO: 4); EQKLISEEDL (SEQ ID NO: 5); YTDIEMNRLGK (SEQ ID NO: 7); KET-AAAKFERQHMDS (SEQ ID NO: 8); GKPIPNPLLGLDST (SEQ ID NO: 9); DYKDDDDK (SEQ ID NO: 1-0); GAPVPYPDPLEPR (SEQ ID NO: 11); HHHHHH (SEQ ID NO: 12); TKENPRSNQEESYDDNES (SEQ ID NO: 13); WSHPQFEK (SEQ ID NO: 14); and PDRVRA-VSHWSS (SEQ ID NO: 15).

16. The method of claim 2, wherein the epitope comprises a length of 200 or less amino acids.

17. The method of claim 16, wherein the epitope comprises a cytokine.

18. The method of claim 17, wherein the cytokine comprises an interleukin (IL).

19. (canceled)

20. The method of claim 17, wherein the cytokine comprises tumor necrosis factor (TNF).

21. The method of claim 17, wherein the cytokine comprises a chemokine (CXC).

22-23. (canceled)

24. The method of claim 2, wherein the epitope comprises a small molecule.

25. (canceled)

26. A composition comprising an antibody recruiting molecule or immune cell recruiting molecule linked to one or more pH-triggered peptides by a non-cleavable linker compound.

27-30. (canceled)

31. A composition comprising the formula of Epitope-Linker-Peptide, wherein Peptide is a pH-triggered peptide comprising the sequence AXDDQNPWRAYLDLLFPTDTLLLDLLWA (SEQ ID NO: 18) or AXDQDNPWRAYLDLLFPTDTLLLDLLWA (SEQ ID NO: 45), wherein upper case “X” indicates any amino acid residue, and can include a lysine, a cysteine, or an Azido-containing amino acid, and wherein Linker is a linker or an extension of the pH-triggered peptide, and wherein each—is a covalent bond.

32. The composition of claim 31, wherein two Epitopes are linked to a single pH-triggered peptide.

33-35. (canceled)

36. The method of claim 1, wherein the subject comprises a solid tumor.

37. The method of claim 1, wherein the subject comprises an inflamed tissue.

38-40. (canceled)

41. The method of claim 1, wherein the construct targets preferentially to a diseased tissue compared to a healthy tissue, thereby minimizing damage to said healthy tissue.

42. (canceled)

43. A method of making a pH-triggered construct to decorate the surface of a diseased cell comprising producing an antibody recruiting molecule or immune cell recruiting molecule linked to one or more pH-triggered peptides by a non-cleavable linker compound, wherein the antibody recruiting molecule or the immune cell recruiting molecule comprises:
(a) an epitope synthesized and linked to a membrane non-inserting part of the pH-triggered peptide;
(b) an epitope synthesized as a membrane non-inserting part of a pH-triggered peptide; or
(c) an epitope expressed as a membrane non-inserting part of a pH-triggered fusion protein.
